(19) United States
(12) Patent Application Publication
(10) Pub. No.: US 2024/0229066 A1
(43) Pub. Date: Jul. 11, 2024

(54) INFECTIOUS RECOMBINANT VESICULAR STOMATITIS VIRUS (RVSV) BEARING THE SPIKE GLYCOPROTEIN S OF SARS-COV-2 AND USES THEREOF

(71) Applicant: Albert Einstein College of Medicine, Bronx, NY (US)

(72) Inventors: Kartik Chandran, Brooklyn, NY (US); Rohit K. Jangra, Shreveport, LA (US); Maria Dieterle, Bronx, NY (US); Denise Haslwanter, Vienna (AT)

(21) Appl. No.: 18/094,117

(22) Filed: Jan. 6, 2023

Related U.S. Application Data
(63) Continuation of application No. PCT/US21/40460, filed on Jul. 6, 2021.
(60) Provisional application No. 63/048,918, filed on Jul. 7, 2020.

(51) Int. Cl. C12N 15/86; A61K 39/215

(52) U.S. Cl. CPC C12N 15/86; A61K 39/215; A61K 2039/5256; C12N 2710/24143

(57) ABSTRACT

Provided herein are recombinant vesicular stomatitis virus (rVSV) or rVSV vector comprising nucleic acid encoding the spike glycoprotein S of SARS-COV-2, compositions comprising such vectors or viruses, as well as screening methods, diagnostic methods, prophylactic and therapeutic methods using such vectors or viruses.

### CROSS-REFERENCE TO RELATED APPLICATION

This application is a continuation of PCT Application No. PCT/US2021/040460, filed Jul. 6, 2021, which claims the benefit of priority to U.S. Provisional Application Ser. No. 63/048,918, filed Jul. 7, 2020. The entire contents of each of said applications are incorporated herein in their entirety by this reference.

### STATEMENT OF RIGHTS

This invention was made with government support under grant numbers U19AI142777, R01AI132633, R01AI143453, R01AI123654, R01AI125462, R21AI141367, and 2T32GM007288-45 awarded by the National Institutes of Health. The government has certain rights in the invention.

### INCORPORATION BY REFERENCE OF MATERIAL SUBMITTED ELECTRONICALLY

The application contains a Sequence Listing that has been submitted electronically in the form of an XML file, created Jan. 26, 2023, and named “AET-01701 SL” (49,672 bytes), the contents of which are incorporated herein by reference in their entirety.

### BACKGROUND OF THE INVENTION

A member of the family Coronaviridae, severe acute respiratory syndrome coronavirus-2 (SARS-CoV-2) is the causative agent of the ongoing coronavirus disease 2019 (COVID-19) pandemic that emerged in Wuhan City, China in late 2019 (Wu et al. (2020a) Nature 579:265-269). With more than 8 million confirmed cases and at least 435,000 deaths in over 216 countries/areas/territories as of Jun. 15, 2020, the global scale and impact of COVID-19 is unparalleled in living memory (Dong et al. (2020) Lancet Infect. Dis. 20:533-534; WHO Situation Report Jun. 15, 2020, available on world wide web who.int/docs/default-source/coronaviruse/situation-reports/20200615-covid-19-sitrep-147.pdf?sfvrsn=2497a605_2). To date, mitigation strategies have relied largely on physical distancing and other public health measures. Although treatments with some small molecule inhibitors and with convalescent plasma have received approvals for emergency use; and vaccines, antivirals, and monoclonal antibodies are being rapidly developed, no FDA-approved countermeasures are currently available.

The membrane-enveloped virions of SARS-CoV-2 are studded with homotrimers of the spike glycoprotein (S), which mediate viral entry into the host cell (Bosch et al (2003) J. Virol. 77:8801-8811; Walls et al (2020) Cell 181:281-292.e6; Wrapp et al (2020) Science 367:1260-1263). S trimers are post-translationally cleaved in the secretory pathway by the proprotein convertase furin to yield N- and C-terminal S1 and S2 subunits, respectively. S1 is organized into an N-terminal domain (NTD), a central receptor-binding domain (RBD), and a C-terminal domain (CTD). S2 bears the hallmarks of a ‘Class I’ membrane fusion subunit, with an N-terminal hydrophobic fusion peptide, N- and C-terminal heptad repeat sequences, a transmembrane domain, and a cytoplasmic tail (Walls et al (2020) Cell 181:281-292.e6; Wrapp et al (2020) Science 367:1260-1263).

The S1 RBD engages the viral receptor, human angiotensin-converting enzyme 2 (hACE2) at the host cell surface (Hoffmann et al. (2020a) Cell 181:271-280.e8; Wang et al. (2020) Cell 181:894-904.e9; Zhou et al. (2020) Nature 579:270-273). Receptor binding is proposed to prime further S protein cleavage at the S2′ site by the transmembrane protease serine protease-2 (TMPRSS2) at the cell surface, and/or by host cysteine cathepsin(s) in endosomes. S2′ cleavage activates S2 conformational rearrangements that catalyze the fusion of viral and cellular membranes and escape of the viral genome into the cytoplasm (Hoffmann et al. (2020a) Cell 181:271-280.e8).

The S glycoprotein is the major antigenic target on the virus for protective antibodies (Rogers et al. (2020) Science doi: 10.1126/science.abc7520; Wec et al. (2020) Science doi: 10.1126/science.abc7424; Wu et al. (2020a) Nature 579:265-269), and is thus of high significance for the development of vaccines and therapeutic antibodies. Plasma derived from COVID-19 human convalescents and replete with such antibodies has shown early promise as a COVID-19 treatment, and it is currently being evaluated in clinical trials of antiviral prophylaxis and therapy (Casadevall and Pirofski (2020) J. Clin. Invest. 130:1545-1548). Considerable efforts are also being aimed at the identification and deployment of S glycoprotein-specific neutralizing monoclonal antibodies (mAbs) (Cao et al. (2020) Cell 182:1-12; Pinto et al. (2020) Nature doi: 10.1038/s41586-020-2349-y; Rogers et al. (2020) Science doi: 10.1126/science.abc7520; Wec et al. (2020) Science doi: 10.1126/science.abc7424; Wu et al. (2020b) Science 368:1274-1278; Zost et al. (2020) BioRxiv 10.1101/2020.05.12.091462). A key requirement for the rapid development of such vaccines and treatments with convalescent plasma, small-molecule inhibitors, and recombinant biologics is the availability of safe, robust, and faithful platforms to study S-glycoprotein inhibition with high assay throughput. Given limited access to biosafety level-3 (BSL-3) containment facilities required to safely handle SARS-CoV-2, researchers have turned to surrogate viral systems that afford studies of cell entry at biosafety level-2 (BSL-2) and facilitate rapid inhibitor screening through the use of fluorescence or luminescence-based reporters. These include retroviruses, lentiviruses, or vesiculoviruses ‘pseudotyped’ with SARS-CoV-2 S and competent for a single round of viral entry and infection (Lei et al. (2020) Nat. Commun. 11:2070; Nie et al. (2020) Emerg. Microbes Infect. 9:680-686; Ou et al. (2020) Nat. Commun. 11:1620; Pu et al. (2020) J. Med. Virol. doi: 10.1002/jmv.25865; Tan et al. (2020) doi: 10.1002/jmv.25865; Xiong et al. (2020) BioRxiv. doi: 10.1101/2020.04.08.026948). However, these single-cycle pseudotyped viruses are typically laborious to produce and challenging to scale up, yield poorly infectious preparations, and suffer background issues in some cases due to contamination with viral particles bearing the orthologous entry glycoprotein (e.g., low levels of vesicular stomatitis virus (VSV) pseudotypes bearing VSV G).

In contrast to the single-cycle pseudotypes, replication-competent recombinant VSVs (rVSVs) encoding the heterologous virus entry glycoprotein gene(s) in cis as their only entry protein(s) are easier to produce at high yields and also afford forward-genetic studies of viral entry. Such rVSVs have been generated and used to safely and effectively study entry by lethal viruses that require high biocontainment (Caí et al. (2019) Front. Microbiol. 10:856; Jae et al. (2013) Science 340:479-483; Jangra et al. (2018) Nature 563:559-563; Kleinfelter et al. (2015) MBio 6:e00801; Maier et al. (2016) Mol. Ther. Nucleic Acids 5:e321; Raaben et al. (2017) Cell Host Microbe 22:688-696.e5; Whelan et al. (1995) Proc Natl Acad Sci USA 92:8388-8392; Wong et al. (2010) J. Virol. 84:163-175) Although rVSVs bearing the S glycoprotein from SARS-CoV (Fukushi et al. (2006) J. Med. Virol. 78:1509-1512; Kapadia et al. (2005) Virology 340:174-182; Kapadia et al. (2008) Virology 376:165-172) and the Middle East respiratory syndrome coronavirus (MERS-CoV) (Liu et al. (2018) Antiviral Res. 150:30-38) have been developed, no such systems have been described to date for SARS-CoV-2.

There is an urgent need for vaccines and therapeutics to prevent and treat COVID-19.

### SUMMARY OF THE INVENTION

The present invention is based, at least in part, on the generation of a rVSV encoding SARS-CoV-2 S and identification of key passage-acquired mutations in the S glycoprotein that facilitate robust rVSV replication. It was shown that the entry-related properties of rVSV-SARS-CoV-2 S closely resemble those of the authentic agent. It was also demonstrated that the neutralization of the rVSV and authentic SARS-CoV-2 by spike-specific antibodies is highly correlated by using a large panel of COVID-19 convalescent sera. These findings underscore the utility of rVSV-SARS-CoV-2 S for the development of spike-specific antivirals and for mechanistic studies of viral entry and its inhibition. Furthermore, rVSV-SARS-CoV-2 S can be used as a diagnostic/serologic test to detect neutralizing antibodies in COVID-19 patient and convalescent samples, and as a candidate live-attenuated COVID-19 vaccine.

In some aspects, provided herein is a recombinant vesicular stomatitis virus (VSV) vector comprising a nucleic acid encoding spike glycoprotein S of severe acute respiratory syndrome coronavirus-2 (SARS-CoV-2), or biologically active fragment thereof.

In some aspects, provided herein is a recombinant vesicular stomatitis virus (VSV) generated from the recombinant VSV vector described herein.

In some aspects, provided herein is a recombinant vesicular stomatitis virus (VSV) comprising a genome which comprises a nucleic acid encoding spike glycoprotein S of SARS-CoV-2, or biologically active fragment thereof.

Numerous embodiments are further provided that can be applied to any aspect of the present invention and/or combined with any other embodiment described herein. For example, in some embodiments, the nucleic acid encoding a wild-type spike glycoprotein S of SARS-CoV-2, or biologically active fragment thereof. In some embodiments, the wild-type spike glycoprotein S of SARS-CoV-2 comprises an amino acid sequence set forth in SEQ ID No: 1. In some embodiments, the nucleic acid comprises a nucleotide sequence set forth in SEQ ID No: 2.

In some embodiments, the nucleic acid encoding a mutated spike glycoprotein S of SARS-CoV-2, or biologically active fragment thereof. In some embodiments, the mutated spike glycoprotein S of SARS-CoV-2 comprises mutations at positions corresponding to C1250, C1253, L517, P812, W64, D253, G261, A372, H655, and/or R685 of SEQ ID NO: 1. In some embodiments, the mutated spike glycoprotein S of SARS-CoV-2 comprises one or more mutations selected from C1250*, C1253*, L517S, P812R, W64R, D253N, G261R, A372T, H655Y, and R685G. In some embodiments, the mutations of the spike glycoprotein S are capable of facilitating replication of rVSV-SARS-CoV-2 S virus. In some embodiments, the mutated spike glycoprotein S of SARS-CoV-2 comprises an amino acid sequence set forth in SEQ ID NOs: 3, 5, 7, 9, 11, or 13. In some embodiments, the nucleic acid comprises a nucleotide sequence set forth in SEQ ID No: 4, 6, 8, 10, 12, or 14. In some embodiments, the mutated spike glycoprotein S of SARS-CoV-2 comprises mutations C1253*, W64R, G261R, and A372T. In some embodiments, the mutated spike glycoprotein S of SARS-CoV-2 comprises an amino acid sequence set forth in SEQ ID No: 7, 9, 11, or 13. In some embodiments, the nucleic acid comprises a nucleotide sequence set forth in SEQ ID No: 8, 10, 12, or 14.

In some embodiments, the recombinant VSV vector or the recombinant VSV genome described herein does not comprise native VSV entry glycoprotein gene, G. In some embodiments, the native VSV entry glycoprotein gene, G, is replaced with the nucleic acid encoding spike glycoprotein S of SARS-CoV-2.

In some embodiments, the recombinant VSV vector or the recombinant VSV genome described herein further comprising a nucleic acid encoding a reporter. In some embodiments, the reporter is a fluorescent protein. In some embodiments, the reporter is an enhanced green fluorescent protein (eGFP).

In some embodiments, the recombinant VSV viral particle incorporates the spike glycoprotein S of SARS-CoV-2. In some embodiments, the recombinant VSV vector or the recombinant VSV is replication-competent. In some embodiments, the recombinant VSV is capable of efficient spread in tissue culture with little or no syncytium formation. In some embodiments, entry of the recombinant VSV in cells depends on level and/or activity of cysteine cathepsins, and/or endosomal acid pH of the cells. In some embodiments, the entry of the recombinant VSV in cells is reduced by NH₄Cl, E-64, and FYdmk. In some embodiments, entry of the recombinant VSV in cells depends on level and/or activity of TMPRSS2 of the cells. In some embodiments, the entry of the recombinant VSV in cells is reduced by camostat mesylate. In some embodiments, entry of the recombinant VSV in cells depends on level and/or activity of ACE2 of the cells, and/or interaction between the spike glycoprotein S and the ACE2 receptor. In some embodiments, the entry of the recombinant VSV in cells is reduced by a receptor-binding domain (RBD) of the spike glycoprotein S or an ACE2-specific antibody. In some embodiments, the ACE2 is a human ACE2. In some embodiments, the cells are of human airway origin.

In some embodiments, the recombinant VSV is attenuated.

In some aspects, provided herein is an immunogenic composition comprising the recombinant VSV vector described herein, or the recombinant VSV described herein. In some embodiments, the immunogenic composition further comprises an adjuvant. In some embodiments, the immunogenic composition further comprises a pharmaceutically acceptable carrier.

In some aspects, provided herein is a kit or device comprising the recombinant VSV vector described herein, or the recombinant VSV described herein. In some embodiments, the immunogenic composition further comprises an adjuvant. In some embodiments, the immunogenic composition further comprises a pharmaceutically acceptable carrier.

In some aspects, provided herein is a method for identifying an agent that prevents and/or treats SARS-CoV-2 infection comprising: (a) contacting cells with the recombinant VSV vector described herein or the recombinant VSV described herein, and a test agent; and (b) identifying the test agent that reduces the recombinant VSV infection of the cells, thereby identifying an agent that prevents and/or treats SARS-CoV-2 infection.

In some embodiments, the step of contacting occurs in vivo, ex vivo, or in vitro. In some embodiments, the test agent is added before, concurrently, or after contacting the cells with the recombinant VSV vector described herein or the recombinant VSV described herein. In some embodiments, the test agent reduces the recombinant VSV infection of the cells compared with that of the cells treated with a control agent. In some embodiments, the test agent reduces the recombinant VSV infection of the cells compared with that of the untreated cells. In some embodiments, the test agent reduces spike glycoprotein-dependent entry of the recombinant VSV. In some embodiments, the test agent reduces interaction between the spike glycoprotein S and the ACE2 receptor. In some embodiments, the recombinant VSV infection is assessed by determining the number or percentage of cells that express the reporter of the recombinant VSV vector or the recombinant VSV. In some embodiments, the number or percentage of cells that express the reporter of the recombinant VSV vector or the recombinant VSV is determined by flow cytometry or microscopy. In some embodiments, the method is conducted in a microplate. In some embodiments, the method is conducted in a high-throughput manner.

In some embodiments, the test agent is a small molecule inhibitor, CRISPR guide RNA (gRNA), RNA interfering agent, oligonucleotide, peptide or peptidomimetic inhibitor, aptamer, antibody, hyperimmune globulin, or intrabody. In some embodiments, the test agent is a small-molecule inhibitor or an antibody targeting the spike glycoprotein S. In some embodiments, the test agent is an anti-spike glycoprotein S antibody. In some embodiments, the test agent is a blood, serum or plasma sample from a COVID-19 convalescent. In some embodiments, the test agent is used to prevent and/or treat SARS-CoV-2 infection. In some embodiments, the method is used in combination with at least one screening assay based on SARS-CoV-2 virus.

In some aspects, provided herein is a method of determining whether a subject has exposure to and/or protection from SARS-CoV-2 comprising: (a) contacting cells with the recombinant VSV vector or the recombinant VSV described herein, and a sample obtained from the subject; and (b) determining the recombinant VSV infection of the cells; wherein the absence of or a lower level of the recombinant VSV infection of the cells compared to a control level indicates that the subject has exposure to and/or protection from SARS-CoV-2.

In some embodiments, the recombinant VSV infection is assessed by determining the number or percentage of cells that express the reporter of the recombinant VSV vector or the recombinant VSV. In some embodiments, the number or percentage of cells that express the reporter of the recombinant VSV vector or the recombinant VSV is determined by flow cytometry or microscopy. In some embodiments, the sample is added before, concurrently, or after contacting the cells with the recombinant VSV vector or the recombinant VSV described herein. In some embodiments, the control level is a reference number. In some embodiments, the control level is a level of the recombinant VSV infection of cells treated with a sample obtained from a subject without exposure to SARS-CoV-2. In some embodiments, the control level is a level of the recombinant VSV infection of untreated cells. In some embodiments, the sample is a blood, serum, or plasma sample.

In some aspects, provided herein is a method of preventing and/or treating SARS-CoV-2 infection in a subject comprising administering to the subject a therapeutically effective amount of an immunogenic composition described herein.

In some embodiments, the immunogenic composition is capable of eliciting an immune response in a subject. In some embodiments, the administered immunogenic composition induces production of antibodies to the spike glycoprotein S of SARS-CoV-2. In some embodiments, the administered immunogenic composition induces an immune response against the SARS-CoV-2 in the subject. In some embodiments, the subject is a mammal. In some embodiments, the mammal is a human, a primate, or a rodent. In some embodiments, the mammal is a human.

### BRIEF DESCRIPTION OF THE DRAWINGS

FIGS. 1A-IE show generation of a recombinant vesicular stomatitis virus (rVSV) bearing the SARS-CoV-2 spike (S) glycoprotein. FIG. 1A shows the schematic representation of the VSV genome, in which its native glycoprotein gene has been replaced by that encoding the SARS-CoV-2 S protein. The VSV genome has been further modified to encode an enhanced green fluorescent protein (eGFP) reporter to easily score for infection. FIG. 1B shows the infectious center formation assay on Vero cells at 24 h post-infection showing growth of the rVSV-SARS-CoV-2 S after the indicated number of rounds of serial passage of the passage #1 virus (carrying wild-type (WT) S sequences) on Huh7.5.1 cell line (scale bar=100 μm). Two representative images for each virus passage, showing infected cells in pseudo-colored in green, from one of the two independent experiments are shown here. FIG. 1C shows that the incorporation of SARS-CoV-2 S into rVSV particles captured on an ELISA plate was detected using antiserum from a COVID-19 convalescent donor (average±SD, n=12 from 3-4 independent experiments). Serum from a COVID-19-negative donor and rVSVs bearing Ebola virus glycoprotein (EBOV GP) were used as negative controls (average±SD, n=6 from 2 independent experiments). FIG. 1D shows representative images showing Vero cells infected with plaque #2, #3 and #6 viruses at 16 h post-infection (scale bar=100 μm). FIG. 1E shows the production of infectious virions at 48 h post-infection from Vero cells infected with the indicated plaque-purified viruses. Titers were measured on Vero cells overexpressing TMPRSS2 (n=4, from two independent titrations).

FIG. 2 shows inhibition of syncytium formation by sera from convalescent donors (related to FIGS. 1A-1E). Vero cells were infected with pre-titrated amounts of rVSV-SARS-CoV-2. At 2 h post-infection, cells were washed with PBS to remove residual virus and then exposed to the indicated dilutions of each convalescent serum. Cells were fixed, their nuclei counterstained, and they were imaged for syncytium formation by eGFP expression at 16 h post-infection. Representative images from one of two independent experiments are shown.

FIG. 3 shows spike missense and nonsense mutations acquired by rVSV-SARS-CoV-2 S during serial passage (related to FIGS. 1A-1E).

FIG. 4 shows locations of selected rVSV-SARS-CoV-2 S mutations in the viral spike glycoprotein (related to FIGS. 1A-1E). Selected mutations (see FIG. 3) are indicated on a structural model of the pre-fusion conformation of the SARS-CoV-2 spike trimer ectodomain in complex with its receptor hACE2 (green). In order to model the SARS-CoV-2 spike trimer bound to hACE, X-ray structures for the SARS-CoV-2 spike trimer (PDB IDs: 6VXX, 6VSB) were combined with the SARS-CoV-2 S1-RBD domain bound to hACE2 (PDB IDs: 6JM0, 6LGZ) by structural superposition using Chimera. Missing loops within the spike protein were modeled using the modeller plugin in Chimera. Domains within one monomer of the spike trimer are depicted in different colors (S1: purple; S1-NTD [N-terminal domain]: orange; S1-RBD: Red; S2: dark blue; S2-fusion peptide [FP]: yellow; S2-S2′ cleavage site: pink). A linear diagram of the spike protein sequence is shown at right. NTD, N-terminal domain, RBD, receptor-binding domain. FP, fusion peptide. See examples for details.

FIGS. 5A-5D show that rVSV-SARS-CoV-2 S infection requires the activity of cysteine cathepsin proteases. FIG. 5A shows that Huh7.5.1 cells pre-treated for 1 hour at 37° C. with the indicated concentrations of NH₄Cl were infected with pre-titrated amounts of rVSVs bearing SARS-CoV-2 S or EBOV GP. Infection was scored by eGFP expression at 16-18 h post-infection (average±SD, n=8 from 2 independent experiments). FIG. 5B shows that Vero cells pre-treated for 90 min at 37° C. with the indicated concentrations of pan-cysteine cathepsin inhibitor E-64 were infected with pre-titrated amounts of rVSVs bearing SARS-CoV-2 S. EBOV GP, or VSV G and scored for infection as above (average±SD, n=6 from 3 independent experiments, except n=4 from 2 independent experiments for EBOV GP). FIG. 5C shows that Vero cells pre-treated for 90 min at 37° C. with the indicated concentrations of cathepsin L/B inhibitor FYdmk were infected with pre-titrated amounts of rVSVs bearing SARS-CoV-2 S, EBOV GP or VSV G. Infection was scored as above (average±SD, n=6 from 3 independent experiments). FIG. 5D shows that Vero cells and Vero cells overexpressing TMPRSS2 pre-treated for 120 min at 37° C. with the indicated concentrations of camostat were infected with pre-titrated amounts of rVSVs bearing SARS-CoV-2 S and subsequently scored for infection. In FIGS. 5B-5D, all comparisons between inhibitor-treated samples were not statistically significant. * p<0.033, ** p<0.002, *** p<0.001.

FIGS. 6A-6E show that rVSV-SARS-CoV-2 S infection requires human ACE2. FIG. 6A shows that Naïve (None) baby hamster kidney (BHK21) cells or cells transduced with a retrovirus carrying human ACE2 cDNA (+hACE2 cDNA) were immunostained for hACE2 expression using an anti-ACE2 antibody. Cells were imaged by fluorescence microscopy. The hACE2 signal is pseudo-colored green (top panel, scale bar=20 μm). These cells were also exposed to serial 5-fold dilutions of rVSV-SARS-CoV-2 S and infection was scored by eGFP expression (bottom panel, scale bar=50 μm). Representative images from one of 3 independent experiments are shown. FIG. 6B shows enumeration of eGFP-positive green cells (Average±SD, n=8 from 3 independent experiments). Red dotted line indicates the assay limit of detection (LOD). FIG. 6C shows that recombinant, Ni-NTA-affinity purified S1-S2 ectodomain (Spike) or the receptor binding domain (RBD) of the SARS-CoV-2 S protein were subjected to SDS-PAGE and Coomassie staining. A representative image from one of two independent purification trials is shown here. FIG. 6D shows that monolayers of Huh7.5.1 cells were pre-incubated with serial 3-fold dilutions of the purified RBD for 1 h at 37° C. and then infected with pre-titrated amounts of rVSVs bearing SARS-CoV-2 S or EBOV GP. At 16-18 h post-infection, cells were fixed, nuclei counter-stained with Hoechst-33342, and infection (eGFP expression) was scored by fluorescence microscopy. It is represented as % relative infection [no RBD=100%, Average±SEM, n=8 from 3-4 (rVSV-SARS-CoV-2 S) or n=4 from 2 (rVSV-EBOV GP) independent experiments]. FIG. 6E shows that monolayers of Huh7.5.1 cells pre-incubated for 1 h at 37° C. with 3-fold serial dilutions of anti-human ACE2 antibody or negative control (hIgG) were infected with pre-titrated amounts of rVSV-SARS-CoV-2 S. Infection was scored as above and is represented as % relative infection [no antibody=100%, Average±SD, n=8 from 3-4 independent experiments]. * p<0.033, ** p<0.002, *** p<0.001.

FIGS. 7A-7E show that rVSV-SARS-CoV-2 S infection in human airway epithelial cells is ACE2-dependent. FIG. 7A shows that infectivity of rVSV-SARS-CoV-2 S was measured in human airway epithelial Calu3 cells and Vero-TMPRSS2 cells by applying serial dilutions of the virus. Infections were scored as described in FIG. 7B (Average±SD, n=4 from two independent titrations). FIGS. 7B-7C show that monolayers of Calu3 cells pre-incubated for 1 h at 37° C. with indicated amounts of anti-human ACE2 antibody or negative control (hIgG) were infected with pre-titrated amounts of rVSV-SARS-CoV-2 S. FIG. 7B shows representative images from one of the two independent experiments are shown (scale bar=100 μm). FIG. 7C shows that infection was scored as above and is represented as % relative infection [no antibody=100%, Average±SD, n=4 from two independent experiments, except for n=2 for hIgG at 100 nM]. FIG. 7D shows that infectivity of rVSV-SARS-CoV-2 S in human respiratory epithelial A549 cells transduced with a retrovirus carrying human ACE2 cDNA or empty vector was evaluated by exposing cells to serial dilutions of rVSV-SARS-CoV-2 S. Infections were scored as described in FIG. 7B (Average±SD, n=6 from 3 independent titrations). Red dotted line indicates the assay limit of detection (LOD). Means were compared by unpaired t-test. FIG. 7E shows that A549 cells transduced as in FIG. 7D were immunostained for hACE2 expression as described in FIG. 7A using an anti-ACE2 antibody. Cells were imaged by fluorescence microscopy. The hACE2 signal is pseudo-colored green and representative images are shown (scale bar=20 μm). * p<0.033, ** p<0.002, *** p<0.001.

FIGS. 8A-8B show rVSV-SARS-CoV-2 S neutralization dose-curves with antisera from convalescent donors (related to FIGS. 9A-9D and FIGS. 10A-10D). Pre-titrated amounts of rVSV-SARS-CoV-2 S were incubated with serial 3-fold dilutions of antisera from two COVID-19 convalescent donors or negative control at 37C for 1 h. Virus:serum mixtures were nuclei were counterstained, P expression (Average±SD, n=4 from 2 independent experiments).

FIGS. 9A-9D show that rVSV-SARS-CoV-2 S neutralization is mediated by S glycoprotein-targeting antibodies in human antisera. FIG. 9A shows that ELISA plates coated with rVSV-SARS-CoV-2 S were incubated with serial 2-fold dilutions of serum 18, serum 39, or negative control serum. Bound S-specific antibodies were detected with an anti-human HRP-conjugated secondary antibody (average±SD, n=4 from 2 independent experiments). FIG. 9B shows the schematic of the antibody depletion study. FIGS. 9C-9D show that pre-titrated amounts of serum 18 and serum 39 were sequentially incubated with SARS-CoV-2 S-coated high-binding plates to deplete S-specific antibodies. Capacity of the depleted sera (and control sera incubated with only the blocking agent) to neutralize rVSV-SARS-CoV-2 S was then estimated by incubating pre-titrated amounts of rVSV at the indicated dilutions of sera at 37° C. for 1 h prior to infecting monolayers of Huh7.5.1 cells. Cells were scored for infection as above (average±SD, n=4 from 2 independent experiments). In FIGS. 9A and 9C-9D, p-values for pairwise comparisons of the untreated sample and inhibitor-treated sample means are shown, unless otherwise indicated. In FIGS. 9C-9D, the depletion #4 and depletion control were compared. * p<0.033, ** p<0.002, *** p<0.001.

FIGS. 10A-10D show the correlation of convalescent serum-mediated neutralization of rVSV-SARS-CoV-2 S and authentic SARS-CoV-2. FIG. 10A shows that pre-titrated amounts of SARS-CoV-2 were incubated with serial 3-fold dilutions of antisera from COVID-19 convalescent donors or negative control at 37° C. for 1 h. Virus-serum mixtures were then applied to monolayers of Vero-E6 cells. At 24 h post-infection, cells were fixed, permeabilized and immunostained with a SARS-CoV nucleocapsid-specific antibody. Nuclei were counterstained, infected cells were scored for the presence of nucleocapsid antigen. Representative images from one of the 2 independent experiments are shown (scale bar=200 μm). FIG. 10B shows that pre-titrated amounts of rVSV-SARS-CoV-2 S were incubated with serial 3-fold dilutions of antisera from COVID-19 convalescent patients or negative control at 37° C. for 1 h. Virus:serum mixtures was then applied to monolayers of Vero cells. At 16-18 hours post-infection, cells were fixed, nuclei were counterstained and infected cells were scored by GFP expression. Heat maps showing % neutralization of authentic SARS-CoV-2 or rVSV-SARS-CoV-2 S by the panel of 40 antisera are shown (Averages of n=4 from 2 independent experiments). FIGS. 10C-10D show the comparison of the neutralizing activities of the antisera (log reciprocal IC₅₀ values) against authentic SARS-CoV-2. and rVSV-SARS-CoV-2 S (‘rVSV SARS2 S’). FIG. 10D shows the linear regression analyses of neutralization IC₅₀ values from FIG. 10C.

FIG. 11 shows the capacities of selected convalescent antisera to neutralize two rVSV-SARS-CoV-2 S passage stocks (related FIGS. 10A-10D). Pre-titrated amounts of rVSV-SARS-CoV-2 S from passage 5 and passage 9 were incubated with serial 3-fold dilutions of antisera from nine COVID-19 convalescent donors or positive and negative controls at 37° C. for 1 hour. Virus:serum mixtures were then applied to monolayers of Vero cells. At 16-18 hours post-infection, cells were fixed, nuclei were counterstained, and infected cells were scored for eGFP expression. IC₅₀ values were extracted from logistic curve fits of the neutralization dose curves and are shown above (±95% confidence intervals).

FIG. 12 shows the schematic of generation and characterization of rVSV-SARS-CoV-2 S.

### DETAILED DESCRIPTION OF THE INVENTION

The present invention is based, at least in part, on the generation of a highly infectious recombinant vesicular stomatitis virus (VSV) bearing the SARS-CoV-2 spike glycoprotein S as its sole entry glycoprotein and the discovery that this recombinant virus, rVSV-SARS-CoV-2 S, closely resembles SARS-CoV-2 in its entry-related properties. The neutralizing activities of a large panel of COVID-19 convalescent sera can be assessed in a high-throughput fluorescent reporter assay with rVSV-SARS-CoV-2 S, and neutralization of rVSV-SARS-CoV-2 S and authentic SARS-CoV-2 by spike-specific antibodies in these antisera is highly correlated. These findings underscore the utility of rVSV-SARS-CoV-2 S for the development of spike-specific therapeutics and for mechanistic studies of viral entry and its inhibition. Furthermore, rVSV-SARS-CoV-2 S can be used as a diagnostic/serologic test to detect neutralizing antibodies in COVID-19 patient and convalescent samples, and as a candidate live-attenuated COVID-19 vaccine.

The present invention provides several technical advantages. Working with authentic SARS-CoV-2 requires a BSL-3 containment laboratory. Access to these facilities are limited, and the necessary enhanced safety precautions limit its broad use. By contrast, the recombinant VSV virus, rVSV-SARS-CoV-2 S, can be safely employed at BSL-2 containment and used for, e.g., diagnostic serology, and screening of neutralizing antibodies and other antiviral drug candidates. Moreover, rVSV-SARS2 S-based neutralization screening can be performed with a reporter (e.g., fluorescent reporter) assay, affording ease of use and assay throughput, whereas screening with the authentic virus requires cumbersome plaque assays or immunofluorescence assays. Finally, because rVSV-SARS2 S is replication-competent, it can be used to identify spike mutations that confer inhibitor resistance through a process of natural selection, thereby allowing the design of robust antiviral drugs or drug combinations that do not engender escape variants.

In addition, there is an acute need for SARS-CoV-2 vaccines. rVSV-SARS-CoV-2 S can serve as a new live-attenuated vaccine. Proof-of-concept for a VSV-vectored vaccine is provided by Ervebo, the FDA-approved Merck vaccine for Ebola virus. The rVSV-SARS-CoV-2 virus disclosed herein bears novel infectivity-enhancing mutations that do not substantially alter its immunogenic properties.

Furthermore, existing VSV-based surrogates for SARS-CoV-2 cell entry are single-cycle vectors capable of only a single round of infection. Therefore, they cannot readily be produced at scale, limiting their utility for large-scale diagnostic serology, drug-screening campaigns, and as a vaccine. This invention describes a replicating virus that can be readily amplified for use and can potentially be manufactured at scale.

Accordingly, the present invention relates, in part, to the recombinant VSV vector and the recombinant VSV virus (rVSV-SARS-CoV-2 S), compositions comprising such recombinant VSV vector or virus, methods for identifying agents that prevent and/or treat SARS-CoV-2 infection, methods of diagnosing subjects exposed to and/or protected from SARS-CoV-2 infection, and methods for preventing and/or treating SARS-CoV-2 infection.

### I. Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “vaccine” refers to a pharmaceutical composition that elicits an immune response to an antigen of interest. The vaccine may also confer protective immunity upon a subject.

“Vector” refers to a nucleic acid molecule (e.g., DNA or RNA) capable of transporting another nucleic acid to which it has been linked. One type of vector is an episome, i.e., a nucleic acid capable of extra-chromosomal replication. Another type of vector may be capable of integrating into the genome of the host cells. In some embodiments, vectors are those capable of autonomous replication and/or expression of nucleic acids to which they are linked. Vectors capable of directing the expression of genes to which they are operatively linked are referred to herein as “expression vectors”. In some embodiments, expression vectors of utility in recombinant DNA techniques are in the form of “plasmids” which refer generally to circular double stranded DNA loops, which, in their vector form are not bound to the chromosome. In some embodiments, vectors (e.g., expression vectors) are in a linearized form. However, as will be appreciated by those skilled in the art, the invention is intended to include such other forms of vectors which serve equivalent functions.

The term “VSV vector” refers to a vector comprising at least a part of a VSV genome or antigenome. VSV vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector. The present invention encompasses VSV vectors that comprise nucleic acid encoding spike glycoprotein S of severe acute respiratory syndrome coronavirus-2 (SARS-CoV-2), or biologically active fragment thereof.

The term “immunotherapeutic agent” may include any molecule, peptide, antibody or other agent which can stimulate a host immune system to generate an immune response to a viral infection in the subject. Various immunotherapeutic agents are useful in the compositions and methods described herein.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

A “kit” is any manufacture (e.g., a package or container) comprising at least one reagent, e.g., a probe or small molecule, for specifically detecting and/or affecting the expression of a marker encompassed by the present invention. The kit may be promoted, distributed, or sold as a unit for performing the methods encompassed by the present invention. The kit may comprise one or more reagents necessary to express a composition useful in the methods encompassed by the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit may be included.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method encompassed by the present invention further comprises obtaining the sample from the individual prior to detecting or determining the presence or level of at least one marker in the sample.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which may be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a viral infection, e.g., SARS-CoV-2 infection. The term “subject” is interchangeable with “patient.”

As used herein, “percent identity” between amino acid sequences is synonymous with “percent homology,” which can be determined using the algorithm of Karlin and Altschul (Proc. Natl. Acad. Sci. USA 87, 2264-2268, 1990), modified by Karlin and Altschul (Proc. Natl. Acad. Sci. USA 90, 5873-5877, 1993). The noted algorithm is incorporated into the NBLAST and XBLAST programs of Altschul et al. (1990) J. Mol. Biol. 215:403-410. BLAST nucleotide searches are performed with the NBLAST program, score=100, wordlength=12, to obtain nucleotide sequences homologous to a polynucleotide described herein. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (1997) Nucleic Acids Res. 25:3389-3402. When utilizing BLAST and Gapped BLAST programs, the default parameters of the respective programs (e.g., XBLAST and NBLAST) are used.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P—NH2) or a mixed phosphoramidate-phosphodiester oligomer. Peyrottes et al. (1996) Nucleic Acids Res. 24: 1841-8; Chaturvedi et al. (1996) Nucleic Acids Res. 24: 2318-23; Schultz et al. (1996) Nucleic Acids Res. 24: 2966-73. A phosphorothioate linkage can be used in place of a phosphodiester linkage. Braun et al. (1988) J. Immunol. 141: 2084-9; Latimer et al. (1995) Molec. Immunol. 32: 1057-1064. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer. Reference to a polynucleotide sequence (such as referring to a SEQ ID NO) also includes the complement sequence.

The following are non-limiting examples of polynucleotides: a gene or gene fragment, exons, introns, genomic RNA, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

A “host cell” includes an individual cell or cell culture which can be or has been a recipient of a VSV vector(s) of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected, transformed or infected in vivo or in vitro with a VSV vector of this invention.

“Replication” and “propagation” are used interchangeably and refer to the ability of an VSV vector of the invention to reproduce or proliferate. These terms are well understood in the art. For purposes of this invention, replication involves production of VSV proteins and is generally directed to reproduction of VSV. Replication can be measured using assays standard in the art. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods encompassed by the present invention may be administered in a sufficient amount to produce a reasonable benefit/risk ratio applicable to such treatment. In certain embodiments, an effective amount of a VSV vector is an amount that is sufficient to palliate, ameliorate, stabilize, reverse, slow or delay the progression of the disease state.

“Treating” a disease in a subject or “treating” a subject having a disease refers to subjecting the subject to a pharmaceutical treatment, e.g., the administration of a drug, such that at least one symptom of the disease is decreased or prevented from worsening.

The term “body fluid” refers to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “determining a suitable treatment regimen for the subject” is taken to mean the determination of a treatment regimen (i.e., a single therapy or a combination of different therapies that are used for the prevention and/or treatment of the viral infection in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “adjuvant” as used herein refers to substances, which when administered prior, together or after administration of an antigen accelerates, prolong and/or enhances the quality and/or strength of an immune response to the antigen in comparison to the administration of the antigen alone. Adjuvants can increase the magnitude and duration of the immune response induced by vaccination.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

“VSV” as used herein refers to any strain of VSV or mutant forms of VSV, such as those described in WO 01/19380 and U.S. patent application publication US20160022748A1. A VSV construct of this invention may be in any of several forms, including, but not limited to, genomic RNA, mRNA, cDNA, part or all of the VSV RNA encapsulated in the nucleocapsid core, VSV complexed with compounds such as PEG and VSV conjugated to a nonviral protein. In some embodiment, VSV vectors of the invention encompasses replication-competent VSV vectors. In some embodiment, VSV vectors comprises a nucleic acid encoding spike glycoprotein S of SARS-CoV-2, or biologically active fragment thereof.

### II. Recombinant VSV Vector and Virus

VSV, a member of the Rhabdoviridae family, is a negative-stranded virus that replicates in the cytoplasm of infected cells, does not undergo genetic recombination or reassortment, has no known transforming potential and does not integrate any part of its genome into the host. VSV comprises an about 11 kilobase genome that encodes for five proteins referred to as the nucleocapsid (N), polymerase proteins (L) and (P), surface glycoprotein (G) and a peripheral matrix protein (M). The genome is tightly encased in nucleocapsid (N) protein and also comprises the polymerase proteins (L) and (P). Following infection of the cell, the polymerase proteins initiate the transcription of five subgenomic viral mRNAs, from the negative-sense genome, that encode the viral proteins. The polymerase proteins are also responsible for the replication of the full-length viral genomes that are packaged into progeny virions. The matrix (M) protein binds to the RNA genome/nucleocapsid core (RNP) and also to the glycosylated (G) protein, which extends from the outer surface in an array of spike like projections and is responsible for binding to cell surface receptors and initiating the infectious process.

Following attachment of VSV through the (G) protein to receptor(s) on the host surface, the virus penetrates the host and uncoats to release the RNP particles. The polymerase proteins, which are carried in with the virus, bind to the 3′ end of the genome and sequentially synthesize the individual mRNAs encoding N, P, M, G, and L, followed by negative-sense progeny genomes. Newly synthesized N, P and L proteins associate in the cytoplasm and form RNP cores which bind to regions of the plasma membrane rich in both M and G proteins. Viral particles form and budding or release of progeny virus ensues.

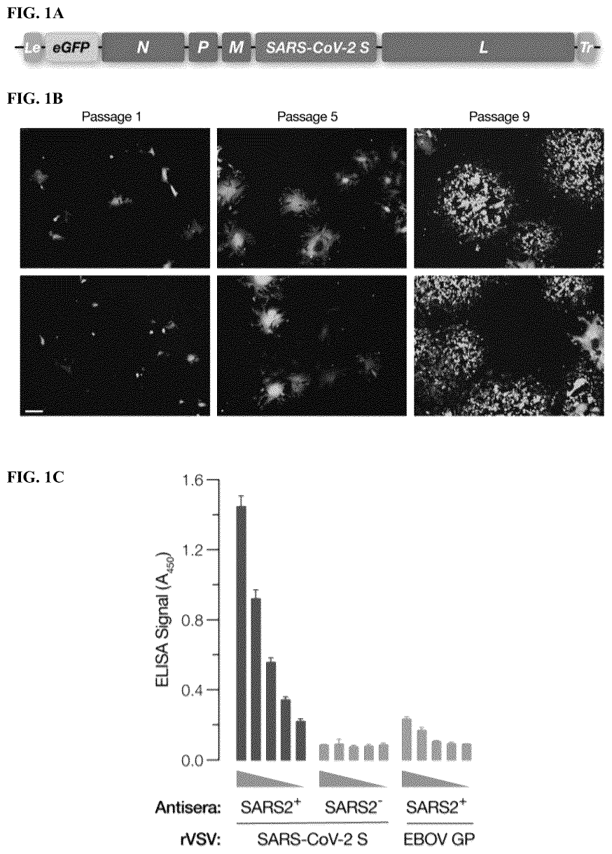

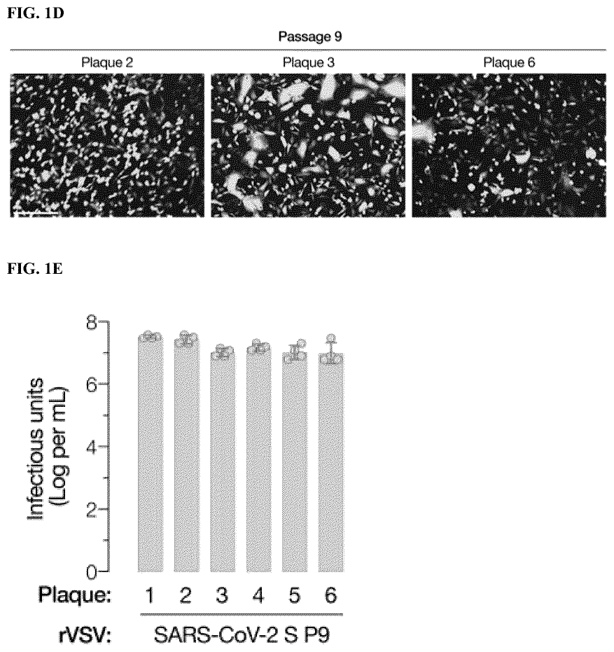

An exemplary schematic illustration of the VSV genome is shown in FIG. 1A. A table of various VSV strains is shown in “Fundamental Virology”, second edition, supra, at page 490. WO 01/19380 and U.S. Pat. No. 6,168,943 disclose that strains of VSV include Indiana, New Jersey. Piry, Colorado, Coccal, Chandipura and San Juan. The complete nucleotide and deduced protein sequence of a VSV genome is known and is available as Genbank VSVCG, accession number J02428; NCBI Seq ID 335873; and is published in Rose and Schubert, 1987, in The Viruses: The Rhabdoviruses, Plenum Press, NY. pp. 129-166. A complete sequence of a VSV strain is shown in U.S. Pat. No. 6,168,943. VSV New Jersey strain is available from the American Type Culture Collection (ATCC) and has ATCC accession number VR-159. VSV Indiana strain is available from the ATCC and has ATCC accession number VR-1421.

In some embodiments, the present invention relates to a nucleic acid comprising at least a part of the VSV genome or antigenome and encoding spike glycoprotein S of SARS-CoV-2 described herein. In certain embodiments, the nucleic acid encodes spike glycoprotein S of SARS-CoV-2 described herein, and N, P, M, and L proteins of a VSV. In certain embodiments, the nucleic acid further comprises a reporter gene.

Typically, said nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Thus, a further object of the invention relates to a vector comprising a nucleic acid of the present invention.

Such vectors can be either circular or linear. Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like. The present invention encompasses the use of any strain of VSV. The present invention encompasses any form of VSV, including, but not limited to genomic RNA, mRNA, cDNA, and part or all of VSV RNA encapsulated in the nucleocapsid core. The present invention encompasses VSV in the form of a VSV vector construct as well as VSV in the form of viral particles. The present invention also encompasses isolated nucleic acid encoding a recombinant VSV vector disclosed herein as well as host cells comprising a recombinant VSV vector of the disclosed herein. In some embodiments, the recombinant VSV vector disclosed herein is replication-competent.

Accordingly, the present invention provides recombinant vesicular stomatitis virus (VSV) vectors comprising nucleic acid encoding spike glycoprotein S of severe acute respiratory syndrome coronavirus-2 (SARS-CoV-2), or biologically active fragment thereof. The exemplary nucleotide sequences and amino acid sequences of the spike glycoprotein S encoded by the recombinant VSV vectors of the present invention are shown in Table 1 below.

TABLE 1
SARS-CoV-2 Spike-Wild type
Amino acid sequence (1273 amino acids) (SEQ ID NO: 1):
MFVFLVLLPLVSSQCVNLTTRTQLPPAYTNSFTRGVYYPDKVFRSSVLHSTQDLFLPFFSNVTWFHAIHV
SGTNGTKRFDNPVLPFNDGVYFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLREFVFKNIDGYFKIYSKHT
PINLVRDLPQGFSALEPLVDLPIGINITRFQTLLALHRSYLTPGDSSSGWTAGAAAYYVGYLQPRTFLLKY
NENGTITDAVDCALDPLSETKCTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGSTPCNGVEGFNC
YFPLQSYGFQPTNGVGYQPYRVVVLSFELLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQDVNCTEVPVAIHA
DQLTPTWRVYSTGSNVFQTRAGCLIGAEHVNNSYECDIPIGAGICASYQTQTNSPRRARSVASQSIIAYT
MSLGAENSVAYSNNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSFIEDLLFNKVTLADAGFIKQYG
DCLGDIAARDLICAQKFNGLTVLPPLLTDEMIAQYTSALLAGTITSGWTFGAGAALQIPFAMQMAYRF
NGIGVTQNVLYENQKLIANQFNSAIGKIQDSLSSTASALGKLQDVVNQNAQALNTLVKQLSSNFGAISS
VLNDILSRLDKVEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDLGDISGINASVVNIQKEI
DRLNEVAKNLNESLIDLQELGKYEQYIKWPWYIWLGFIAGLIAIVMVTIMLCCMTSCCSCLKGCCSCGS
CCKFDEDDSEPVLKGVKLHYT*
Nucleotide sequence (SEQ ID NO: 2):
ATGTTCGTGTTCCTGGTGCTCCTGCCTCTGGTGAGCAGCCAGTGCGTGAACCTGACCACCCGAACC
CAGCTCCCACCAGCCTACACCAACAGCTTTACACGGGGCGTGTACTACCCTGACAAGGTGTTCAGA
TCTAGCGTCCTGCACAGCACTCAGGACCTCTTCCTGCCGTTCTTCAGCAACGTGACATGGTTCCACG
CCATCCACGTGAGCGGCACAAACGGAACCAAGCGGTTTGATAACCCCGTCCTGCCATTCAATGAT
GGAGTTTACTTCGCCAGTACCGAGAAGAGTAACATCATCCGGGGCTGGATCTTCGGCACCACCCT
GGATAGCAAAACACAGAGCCTCCTGATCGTGAACAATGCCACGAACGTCGTGATCAAGGTGTGCG
AGTTCCAGTTTTGCAATGATCCTTTCCTGGGTGTGTACTACCACAAGAACAACAAGAGCTGGATGG
AAAGCGAGTTCAGAGTCTACAGCAGCGCCAACAACTGCACATTCGAGTACGTCTCTCAGCCTTTTC
TGATGGACCTTGAGGGGAAACAAGGCAACTTCAAGAACCTGAGAGAATTCGTGTTCAAGAACATC
GACGGCTACTTCAAAATCTACTCCAAGCACACACCCATCAACCTGGTCCGGGACCTCCCTCAGGGC
TTCAGCGCCCTGGAACCCCTGGTCGACCTGCCCATAGGCATCAACATAACGCGGTTCCAAACCCTG
CTGGCCCTGCATAGATCCTACCTGACTCCTGGCGACAGCAGCAGCGGATGGACCGCCGGAGCTGC
AGCCTACTATGTGGGCTACCTGCAACCTAGAACCTTCCTGCTGAAGTACAACGAGAACGGCACAAT
CACAGACGCCGTCGACTGCGCCCTGGACCCTCTCTCTGAGACAAAGTGCACCCTGAAGTCCTTCAC
CGTGGAAAAGGGCATCTACCAGACCAGCAACTTCCGGGTGCAGCCTACAGAGAGCATCGTGCGAT
TTCCAAACATTACCAACCTCTGCCCCTTCGGCGAGGTGTTTAACGCCACAAGATTTGCCTCCGTTTA
CGCCTGGAATAGAAAGAGAATCAGCAATTGTGTGGCCGACTACTCCGTGCTGTATAACAGCGCCT
CTTTCAGCACCTTCAAGTGCTACGGCGTTTCCCCAACAAAGCTGAATGACCTGTGCTTCACCAACGT
GTACGCCGACTCCTTCGTAATTAGAGGCGATGAGGTGCGGCAGATCGCACCAGGCCAGACCGGTA
AGATCGCTGACTACAACTATAAGCTGCCTGATGATTTTACAGGCTGCGTGATCGCCTGGAACTCTA
ACAACCTGGATAGCAAGGTGGGCGGCAACTACAACTACCTGTACCGGCTGTTTCGCAAGTCTAAC
CTGAAACCTTTCGAGAGAGACATCTCCACAGAGATCTACCAGGCCGGTTCTACACCTTGTAACGGG
GTGGAAGGCTTCAACTGTTACTTCCCTCTGCAAAGCTACGGCTTCCAGCCTACCAATGGAGTCGGC
TACCAGCCATACCGGGTGGTCGTGCTGTCCTTCGAGTTACTCCACGCCCCCGCCACCGTCTGCGGT
CCTAAGAAGTCCACCAATCTGGTTAAGAACAAATGCGTGAACTTCAACTTCAACGGCCTGACCGG
GACCGGCGTGCTGACCGAAAGCAACAAAAAGTTCCTCCCCTTCCAGCAGTTCGGCCGTGATATCG
CTGACACCACAGATGCCGTCAGAGATCCACAGACCCTGGAAATCCTGGATATTACACCCTGCTCCT
TCGGAGGAGTTTCTGTGATCACCCCCGGGACCAATACCAGCAACCAGGTGGCTGTGCTGTACCAA
GATGTTAACTGCACCGAGGTTCCTGTGGCCATCCACGCCGATCAGCTGACACCTACTTGGAGAGT
GTACTCCACTGGCTCCAATGTGTTCCAGACCAGGGCCGGATGTCTGATCGGCGCCGAGCACGTGA
ATAACAGTTACGAGTGCGACATCCCTATCGGCGCCGGCATCTGTGCCAGCTACCAGACCCAGACA
AACAGCCCTAGACGGGCTAGATCTGTAGCTAGCCAGAGCATCATCGCCTACACCATGAGCCTGGG
CGCAGAGAACAGCGTGGCCTATTCCAACAACTCTATCGCCATTCCCACCAACTTTACAATTAGCGTC
ACAACAGAGATCCTGCCCGTGAGCATGACCAAGACCAGCGTGGACTGTACAATGTACATCTGTGG
CGACAGCACTGAATGCAGCAACCTGCTGCTGCAATACGGCTCCTTTTGCACCCAACTGAACCGGGC
GCTGACCGGAATCGCCGTGGAACAGGACAAAAATACCCAGGAGGTGTTCGCCCAAGTGAAGCAG
ATCTACAAGACCCCACCTATCAAGGACTTCGGCGGCTTTAACTTTAGCCAGATTCTCCCTGATCCTT
CTAAGCCTAGCAAGCGGAGCTTTATCGAGGATCTGCTGTTCAACAAGGTCACCCTGGCCGATGCC
GGCTTTATCAAACAGTATGGCGATTGCCTGGGCGACATAGCCGCCAGAGATCTGATCTGCGCCCA
GAAATTCAACGGCCTGACAGTTCTCCCACCTCTGCTGACCGACGAGATGATCGCTCAGTACACCTC
TGCCCTGCTGGCTGGCACCATCACATCTGGGTGGACATTTGGCGCCGGCGCCGCCCTGCAGATCC
CCTTTGCCATGCAGATGGCCTATAGATTCAACGGAATCGGCGTGACCCAGAACGTGCTGTATGAA
AACCAGAAGCTGATCGCTAACCAGTTCAATTCTGCCATCGGCAAGATCCAGGACTCCCTCTCCTCT
ACCGCCAGCGCCCTGGGCAAACTGCAGGACGTGGTGAATCAGAACGCCCAAGCCCTGAACACCCT
GGTGAAGCAGCTCAGCAGCAATTTTGGCGCCATCAGCTCTGTGCTGAACGATATCCTGTCTAGACT
GGACAAGGTGGAAGCCGAAGTCCAGATCGATAGACTGATCACAGGCAGACTGCAGTCCCTGCAA
ACCTACGTGACCCAACAGCTGATCAGGGCCGCTGAAATAAGAGCCAGCGCCAATCTCGCCGCTAC
CAAGATGTCCGAGTGTGTGCTGGGACAGTCTAAACGCGTTGACTTCTGCGGCAAAGGCTATCACC
TGATGAGCTTCCCCCAGAGCGCGCCGCACGGCGTGGTGTTCCTGCATGTGACATACGTGCCTGCC
CAAGAGAAGAATTTCACAACCGCCCCTGCCATCTGCCACGACGGCAAGGCCCACTTCCCAAGAGA
GGGCGTTTTCGTTTCCAATGGCACACACTGGTTCGTGACACAAAGAAACTTCTACGAACCCCAGAT
TATCACCACCGACAACACCTTCGTGAGTGGCAATTGTGACGTGGTCATCGGAATCGTGAACAACAC
AGTGTACGACCCTCTGCAACCTGAGCTGGACTCTTTTAAGGAAGAGCTGGACAAGTACTTTAAAAA
CCACACCAGCCCCGATGTGGACCTGGGCGACATCAGTGGCATTAACGCCAGCGTGGTGAACATCC
AAAAGGAAATCGACAGACTGAACGAGGTGGCCAAGAACCTGAACGAGTCCCTGATCGACCTGCA
GGAGCTCGGCAAATACGAGCAGTACATCAAGTGGCCTTGGTACATCTGGCTGGGATTCATCGCCG
GACTGATCGCCATCGTGATGGTGACCATCATGCTGTGCTGTATGACCAGCTGCTGCAGTTGCCTGA
AGGGCTGTTGCAGCTGCGGCAGCTGCTGCAAGTTCGACGAAGATGACTCTGAGCCTGTGCTGAAG
GGCGTGAAGCTGCACTACACCTGA
SARS-CoV-2 Spike-Passage-5
Amino acid sequence (showing changes in bold and underlined) (SEQ ID NO: 3):
Mutations: A372T, L517S, P812R, C1250* and C1253*
MFVFLVLLPLVSSQCVNLTTRTQLPPAYTNSFTRGVYYPDKVFRSSVLHSTQDLFLPFFSNVTWFHAIHV
SGTNGTKRFDNPVLPFNDGVYFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLREFVFKNIDGYFKIYSKHT
PINLVRDLPQGFSALEPLVDLPIGINITRFQTLLALHRSYLTPGDSSSGWTAGAAAYYVGYLQPRTFLLKY
NENGTITDAVDCALDPLSETKCTLKSFTVEKGIYQTSNFRVQPTESIVRFPNITNLCPFGEVFNATRFASV
YAWNRKRISNCVADYSVLYNSTSFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGSTPCNGVEGFNC
YFPLQSYGFQPTNGVGYQPYRVVVLSFESLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQDVNCTEVPVAIHA
DQLTPTWRVYSTGSNVFQTRAGCLIGAEHVNNSYECDIPIGAGICASYQTQTNSPRRARSVASQSIIAYT
MSLGAENSVAYSNNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKRSKRSFIEDLLFNKVTLADAGFIKQYG
DCLGDIAARDLICAQKFNGLTVLPPLLTDEMIAQYTSALLAGTITSGWTFGAGAALQIPFAMQMAYRF
NGIGVTQNVLYENQKLIANQFNSAIGKIQDSLSSTASALGKLQDVVNQNAQALNTLVKQLSSNFGAISS
VLNDILSRLDKVEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDLGDISGINASVVNIQKEI
DRLNEVAKNLNESLIDLQELGKYEQYIKWPWYIWLGFIAGLIAIVMVTIMLCCMTSCCSCLKGCCS*GS
*CKFDEDDSEPVLKGVKLHYT*
Nucleotide sequence (showing changes in bold lowercase) (SEQ ID NO: 4):
ATGTTCGTGTTCCTGGTGCTCCTGCCTCTGGTGAGCAGCCAGTGCGTGAACCTGACCACCCGAACC
CAGCTCCCACCAGCCTACACCAACAGCTTTACACGGGGCGTGTACTACCCTGACAAGGTGTTCAGA
TCTAGCGTCCTGCACAGCACTCAGGACCTCTTCCTGCCGTTCTTCAGCAACGTGACATGGTTCCACG
CCATCCACGTGAGCGGCACAAACGGAACCAAGCGGTTTGATAACCCCGTCCTGCCATTCAATGAT
GGAGTTTACTTCGCCAGTACCGAGAAGAGTAACATCATCCGGGGCTGGATCTTCGGCACCACCCT
GGATAGCAAAACACAGAGCCTCCTGATCGTGAACAATGCCACGAACGTCGTGATCAAGGTGTGCG
AGTTCCAGTTTTGCAATGATCCTTTCCTGGGTGTGTACTACCACAAGAACAACAAGAGCTGGATGG
AAAGCGAGTTCAGAGTCTACAGCAGCGCCAACAACTGCACATTCGAGTACGTCTCTCAGCCTTTTC
TGATGGACCTTGAGGGGAAACAAGGCAACTTCAAGAACCTGAGAGAATTCGTGTTCAAGAACATC
GACGGCTACTTCAAAATCTACTCCAAGCACACACCCATCAACCTGGTCCGGGACCTCCCTCAGGGC
TTCAGCGCCCTGGAACCCCTGGTCGACCTGCCCATAGGCATCAACATAACGCGGTTCCAAACCCTG
CTGGCCCTGCATAGATCCTACCTGACTCCTGGCGACAGCAGCAGCGGATGGACCGCCGGAGCTGC
AGCCTACTATGTGGGCTACCTGCAACCTAGAACCTTCCTGCTGAAGTACAACGAGAACGGCACAAT
CACAGACGCCGTCGACTGCGCCCTGGACCCTCTCTCTGAGACAAAGTGCACCCTGAAGTCCTTCAC
CGTGGAAAAGGGCATCTACCAGACCAGCAACTTCCGGGTGCAGCCTACAGAGAGCATCGTGCGAT
TTCCAAACATTACCAACCTCTGCCCCTTCGGCGAGGTGTTTAACGCCACAAGATTTGCCTCCGTTTA
CGCCTGGAATAGAAAGAGAATCAGCAATTGTGTGGCCGACTACTCCGTGCTGTATAACAGCaCCT
CTTTCAGCACCTTCAAGTGCTACGGCGTTTCCCCAACAAAGCTGAATGACCTGTGCTTCACCAACGT
GTACGCCGACTCCTTCGTAATTAGAGGCGATGAGGTGCGGCAGATCGCACCAGGCCAGACCGGTA
AGATCGCTGACTACAACTATAAGCTGCCTGATGATTTTACAGGCTGCGTGATCGCCTGGAACTCTA
ACAACCTGGATAGCAAGGTGGGCGGCAACTACAACTACCTGTACCGGCTGTTTCGCAAGTCTAAC
CTGAAACCTTTCGAGAGAGACATCTCCACAGAGATCTACCAGGCCGGTTCTACACCTTGTAACGGG
GTGGAAGGCTTCAACTGTTACTTCCCTCTGCAAAGCTACGGCTTCCAGCCTACCAATGGAGTCGGC
TACCAGCCATACCGGGTGGTCGTGCTGTCCTTCGAGTcACTCCACGCCCCCGCCACCGTCTGCGGT
CCTAAGAAGTCCACCAATCTGGTTAAGAACAAATGCGTGAACTTCAACTTCAACGGCCTGACCGG
GACCGGCGTGCTGACCGAAAGCAACAAAAAGTTCCTCCCCTTCCAGCAGTTCGGCCGTGATATCG
CTGACACCACAGATGCCGTCAGAGATCCACAGACCCTGGAAATCCTGGATATTACACCCTGCTCCT
TCGGAGGAGTTTCTGTGATCACCCCCGGGACCAATACCAGCAACCAGGTGGCTGTGCTGTACCAA
GATGTTAACTGCACCGAGGTTCCTGTGGCCATCCACGCCGATCAGCTGACACCTACTTGGAGAGT
GTACTCCACTGGCTCCAATGTGTTCCAGACCAGGGCCGGATGTCTGATCGGCGCCGAGCACGTGA
ATAACAGTTACGAGTGCGACATCCCTATCGGCGCCGGCATCTGTGCCAGCTACCAGACCCAGACA
AACAGCCCTAGACGGGCTAGATCTGTAGCTAGCCAGAGCATCATCGCCTACACCATGAGCCTGGG
CGCAGAGAACAGCGTGGCCTATTCCAACAACTCTATCGCCATTCCCACCAACTTTACAATTAGCGTC
ACAACAGAGATCCTGCCCGTGAGCATGACCAAGACCAGCGTGGACTGTACAATGTACATCTGTGG
CGACAGCACTGAATGCAGCAACCTGCTGCTGCAATACGGCTCCTTTTGCACCCAACTGAACCGGGC
GCTGACCGGAATCGCCGTGGAACAGGACAAAAATACCCAGGAGGTGTTCGCCCAAGTGAAGCAG
ATCTACAAGACCCCACCTATCAAGGACTTCGGCGGCTTTAACTTTAGCCAGATTCTCCCTGATCCTT
CTAAGCgTAGCAAGCGGAGCTTTATCGAGGATCTGCTGTTCAACAAGGTCACCCTGGCCGATGCC
GGCTTTATCAAACAGTATGGCGATTGCCTGGGCGACATAGCCGCCAGAGATCTGATCTGCGCCCA
GAAATTCAACGGCCTGACAGTTCTCCCACCTCTGCTGACCGACGAGATGATCGCTCAGTACACCTC
TGCCCTGCTGGCTGGCACCATCACATCTGGGTGGACATTTGGCGCCGGCGCCGCCCTGCAGATCC
CCTTTGCCATGCAGATGGCCTATAGATTCAACGGAATCGGCGTGACCCAGAACGTGCTGTATGAA
AACCAGAAGCTGATCGCTAACCAGTTCAATTCTGCCATCGGCAAGATCCAGGACTCCCTCTCCTCT
ACCGCCAGCGCCCTGGGCAAACTGCAGGACGTGGTGAATCAGAACGCCCAAGCCCTGAACACCCT
GGTGAAGCAGCTCAGCAGCAATTTTGGCGCCATCAGCTCTGTGCTGAACGATATCCTGTCTAGACT
GGACAAGGTGGAAGCCGAAGTCCAGATCGATAGACTGATCACAGGCAGACTGCAGTCCCTGCAA
ACCTACGTGACCCAACAGCTGATCAGGGCCGCTGAAATAAGAGCCAGCGCCAATCTCGCCGCTAC
CAAGATGTCCGAGTGTGTGCTGGGACAGTCTAAACGCGTTGACTTCTGCGGCAAAGGCTATCACC
TGATGAGCTTCCCCCAGAGCGCGCCGCACGGCGTGGTGTTCCTGCATGTGACATACGTGCCTGCC
CAAGAGAAGAATTTCACAACCGCCCCTGCCATCTGCCACGACGGCAAGGCCCACTTCCCAAGAGA
GGGCGTTTTCGTTTCCAATGGCACACACTGGTTCGTGACACAAAGAAACTTCTACGAACCCCAGAT
TATCACCACCGACAACACCTTCGTGAGTGGCAATTGTGACGTGGTCATCGGAATCGTGAACAACAC
AGTGTACGACCCTCTGCAACCTGAGCTGGACTCTTTTAAGGAAGAGCTGGACAAGTACTTTAAAAA
CCACACCAGCCCCGATGTGGACCTGGGCGACATCAGTGGCATTAACGCCAGCGTGGTGAACATCC
AAAAGGAAATCGACAGACTGAACGAGGTGGCCAAGAACCTGAACGAGTCCCTGATCGACCTGCA
GGAGCTCGGCAAATACGAGCAGTACATCAAGTGGCCTTGGTACATCTGGCTGGGATTCATCGCCG
GACTGATCGCCATCGTGATGGTGACCATCATGCTGTGCTGTATGACCAGCTGCTGCAGTTGCCTGA
AGGGCTGTTGCAGCTGaGGCAGCTGaTGCAAGTTCGACGAAGATGACTCTGAGCCTGTGCTGAAG
GGCGTGAAGCTGCACTACACCTGA
SARS-CoV-2 Spike-Passages 7, 8 and 9
Amino acid sequence (showing changes in bold and underlined)
(SEQ ID NO: 5 and SEQ ID NO: 15):
Mutations: W64R, G261R, A372T, L517S, P812R, C1250* and C1253*
MFVFLVLLPLVSSQCVNLTTRTQLPPAYTNSFTRGVYYPDKVFRSSVLHSTQDLFLPFFSNVTRFHAIHV
SGTNGTKRFDNPVLPFNDGVYFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLREFVFKNIDGYFKIYSKHT
PINLVRDLPQGFSALEPLVDLPIGINITRFQTLLALHRSYLTPGDSSSGWTARAAAYYVGYLQPRTFLLKY
NENGTITDAVDCALDPLSETKCTLKSFTVEKGIYQTSNFRVQPTESIVRFPNITNLCPFGEVFNATRFASV
YAWNRKRISNCVADYSVLYNSTSFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGSTPCNGVEGFNC
YFPLQSYGFQPTNGVGYQPYRVVVLSFESLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQDVNCTEVPVAIHA
DQLTPTWRVYSTGSNVFQTRAGCLIGAEHVNNSYECDIPIGAGICASYQTQTNSPRRARSVASQSIIAYT
MSLGAENSVAYSNNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKRSKRSFIEDLLFNKVTLADAGFIKQYG
DCLGDIAARDLICAQKFNGLTVLPPLLTDEMIAQYTSALLAGTITSGWTFGAGAALQIPFAMQMAYRF
NGIGVTQNVLYENQKLIANQFNSAIGKIQDSLSSTASALGKLQDVVNQNAQALNTLVKQLSSNFGAISS
VLNDILSRLDKVEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDLGDISGINASVVNIQKEI
DRLNEVAKNLNESLIDLQELGKYEQYIKWPWYIWLGFIAGLIAIVMVTIMLCCMTSCCSCLKGCCS*GS
*CKFDEDDSEPVLKGVKLHYT*
Nucleotide sequence (showing changes in bold lowercase) (SEQ ID NO: 6):
ATGTTCGTGTTCCTGGTGCTCCTGCCTCTGGTGAGCAGCCAGTGCGTGAACCTGACCACCCGAACC
CAGCTCCCACCAGCCTACACCAACAGCTTTACACGGGGCGTGTACTACCCTGACAAGGTGTTCAGA
TCTAGCGTCCTGCACAGCACTCAGGACCTCTTCCTGCCGTTCTTCAGCAACGTGACAcGGTTCCACG
CCATCCACGTGAGCGGCACAAACGGAACCAAGCGGTTTGATAACCCCGTCCTGCCATTCAATGAT
GGAGTTTACTTCGCCAGTACCGAGAAGAGTAACATCATCCGGGGCTGGATCTTCGGCACCACCCT
GGATAGCAAAACACAGAGCCTCCTGATCGTGAACAATGCCACGAACGTCGTGATCAAGGTGTGCG
AGTTCCAGTTTTGCAATGATCCTTTCCTGGGTGTGTACTACCACAAGAACAACAAGAGCTGGATGG
AAAGCGAGTTCAGAGTCTACAGCAGCGCCAACAACTGCACATTCGAGTACGTCTCTCAGCCTTTTC
TGATGGACCTTGAGGGGAAACAAGGCAACTTCAAGAACCTGAGAGAATTCGTGTTCAAGAACATC
GACGGCTACTTCAAAATCTACTCCAAGCACACACCCATCAACCTGGTCCGGGACCTCCCTCAGGGC
TTCAGCGCCCTGGAACCCCTGGTCGACCTGCCCATAGGCATCAACATAACGCGGTTCCAAACCCTG
CTGGCCCTGCATAGATCCTACCTGACTCCTGGCGACAGCAGCAGCGGATGGACCGCCaGAGCTGC
AGCCTACTATGTGGGCTACCTGCAACCTAGAACCTTCCTGCTGAAGTACAACGAGAACGGCACAAT
CACAGACGCCGTCGACTGCGCCCTGGACCCTCTCTCTGAGACAAAGTGCACCCTGAAGTCCTTCAC
CGTGGAAAAGGGCATCTACCAGACCAGCAACTTCCGGGTGCAGCCTACAGAGAGCATCGTGCGAT
TTCCAAACATTACCAACCTCTGCCCCTTCGGCGAGGTGTTTAACGCCACAAGATTTGCCTCCGTTTA
CGCCTGGAATAGAAAGAGAATCAGCAATTGTGTGGCCGACTACTCCGTGCTGTATAACAGCaCCT
CTTTCAGCACCTTCAAGTGCTACGGCGTTTCCCCAACAAAGCTGAATGACCTGTGCTTCACCAACGT
GTACGCCGACTCCTTCGTAATTAGAGGCGATGAGGTGCGGCAGATCGCACCAGGCCAGACCGGTA
AGATCGCTGACTACAACTATAAGCTGCCTGATGATTTTACAGGCTGCGTGATCGCCTGGAACTCTA
ACAACCTGGATAGCAAGGTGGGCGGCAACTACAACTACCTGTACCGGCTGTTTCGCAAGTCTAAC
CTGAAACCTTTCGAGAGAGACATCTCCACAGAGATCTACCAGGCCGGTTCTACACCTTGTAACGGG
GTGGAAGGCTTCAACTGTTACTTCCCTCTGCAAAGCTACGGCTTCCAGCCTACCAATGGAGTCGGC
TACCAGCCATACCGGGTGGTCGTGCTGTCCTTCGAGTcACTCCACGCCCCCGCCACCGTCTGCGGT
CCTAAGAAGTCCACCAATCTGGTTAAGAACAAATGCGTGAACTTCAACTTCAACGGCCTGACCGG
GACCGGCGTGCTGACCGAAAGCAACAAAAAGTTCCTCCCCTTCCAGCAGTTCGGCCGTGATATCG
CTGACACCACAGATGCCGTCAGAGATCCACAGACCCTGGAAATCCTGGATATTACACCCTGCTCCT
TCGGAGGAGTTTCTGTGATCACCCCCGGGACCAATACCAGCAACCAGGTGGCTGTGCTGTACCAA
GATGTTAACTGCACCGAGGTTCCTGTGGCCATCCACGCCGATCAGCTGACACCTACTTGGAGAGT
GTACTCCACTGGCTCCAATGTGTTCCAGACCAGGGCCGGATGTCTGATCGGCGCCGAGCACGTGA
ATAACAGTTACGAGTGCGACATCCCTATCGGCGCCGGCATCTGTGCCAGCTACCAGACCCAGACA
AACAGCCCTAGACGGGCTAGATCTGTAGCTAGCCAGAGCATCATCGCCTACACCATGAGCCTGGG
CGCAGAGAACAGCGTGGCCTATTCCAACAACTCTATCGCCATTCCCACCAACTTTACAATTAGCGTC
ACAACAGAGATCCTGCCCGTGAGCATGACCAAGACCAGCGTGGACTGTACAATGTACATCTGTGG
CGACAGCACTGAATGCAGCAACCTGCTGCTGCAATACGGCTCCTTTTGCACCCAACTGAACCGGGC
GCTGACCGGAATCGCCGTGGAACAGGACAAAAATACCCAGGAGGTGTTCGCCCAAGTGAAGCAG
ATCTACAAGACCCCACCTATCAAGGACTTCGGCGGCTTTAACTTTAGCCAGATTCTCCCTGATCCTT
CTAAGCgTAGCAAGCGGAGCTTTATCGAGGATCTGCTGTTCAACAAGGTCACCCTGGCCGATGCC
GGCTTTATCAAACAGTATGGCGATTGCCTGGGCGACATAGCCGCCAGAGATCTGATCTGCGCCCA
GAAATTCAACGGCCTGACAGTTCTCCCACCTCTGCTGACCGACGAGATGATCGCTCAGTACACCTC
TGCCCTGCTGGCTGGCACCATCACATCTGGGTGGACATTTGGCGCCGGCGCCGCCCTGCAGATCC
CCTTTGCCATGCAGATGGCCTATAGATTCAACGGAATCGGCGTGACCCAGAACGTGCTGTATGAA
AACCAGAAGCTGATCGCTAACCAGTTCAATTCTGCCATCGGCAAGATCCAGGACTCCCTCTCCTCT
ACCGCCAGCGCCCTGGGCAAACTGCAGGACGTGGTGAATCAGAACGCCCAAGCCCTGAACACCCT
GGTGAAGCAGCTCAGCAGCAATTTTGGCGCCATCAGCTCTGTGCTGAACGATATCCTGTCTAGACT
GGACAAGGTGGAAGCCGAAGTCCAGATCGATAGACTGATCACAGGCAGACTGCAGTCCCTGCAA
ACCTACGTGACCCAACAGCTGATCAGGGCCGCTGAAATAAGAGCCAGCGCCAATCTCGCCGCTAC
CAAGATGTCCGAGTGTGTGCTGGGACAGTCTAAACGCGTTGACTTCTGCGGCAAAGGCTATCACC
TGATGAGCTTCCCCCAGAGCGCGCCGCACGGCGTGGTGTTCCTGCATGTGACATACGTGCCTGCC
CAAGAGAAGAATTTCACAACCGCCCCTGCCATCTGCCACGACGGCAAGGCCCACTTCCCAAGAGA
GGGCGTTTTCGTTTCCAATGGCACACACTGGTTCGTGACACAAAGAAACTTCTACGAACCCCAGAT
TATCACCACCGACAACACCTTCGTGAGTGGCAATTGTGACGTGGTCATCGGAATCGTGAACAACAC
AGTGTACGACCCTCTGCAACCTGAGCTGGACTCTTTTAAGGAAGAGCTGGACAAGTACTTTAAAAA
CCACACCAGCCCCGATGTGGACCTGGGCGACATCAGTGGCATTAACGCCAGCGTGGTGAACATCC
AAAAGGAAATCGACAGACTGAACGAGGTGGCCAAGAACCTGAACGAGTCCCTGATCGACCTGCA
GGAGCTCGGCAAATACGAGCAGTACATCAAGTGGCCTTGGTACATCTGGCTGGGATTCATCGCCG
GACTGATCGCCATCGTGATGGTGACCATCATGCTGTGCTGTATGACCAGCTGCTGCAGTTGCCTGA
AGGGCTGTTGCAGCTGaGGCAGCTGaTGCAAGTTCGACGAAGATGACTCTGAGCCTGTGCTGAAG
GGCGTGAAGCTGCACTACACCTGA
SARS-CoV-2 Spike-Plaque #1 and #2
Amino acid sequence (showing changes in bold and underlined)
(SEQ ID NO: 7 and SEQ ID NO: 15):
Mutations: W64R, G261R, A372T, H655Y, R685G and C1253*
MFVFLVLLPLVSSQCVNLTTRTQLPPAYTNSFTRGVYYPDKVFRSSVLHSTQDLFLPFFSNVTRFHAIHV
SGTNGTKRFDNPVLPFNDGVYFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLREFVFKNIDGYFKIYSKHT
PINLVRDLPQGFSALEPLVDLPIGINITRFQTLLALHRSYLTPGDSSSGWTARAAAYYVGYLQPRTFLLKY
NENGTITDAVDCALDPLSETKCTLKSFTVEKGIYQTSNFRVQPTESIVRFPNITNLCPFGEVFNATRFASV
YAWNRKRISNCVADYSVLYNSTSFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGSTPCNGVEGFNC
YFPLQSYGFQPTNGVGYQPYRVVVLSFELLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQDVNCTEVPVAIHA
DQLTPTWRVYSTGSNVFQTRAGCLIGAEYVNNSYECDIPIGAGICASYQTQTNSPRRAGSVASQSIIAYT
MSLGAENSVAYSNNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSFIEDLLFNKVTLADAGFIKQYG
DCLGDIAARDLICAQKFNGLTVLPPLLTDEMIAQYTSALLAGTITSGWTFGAGAALQIPFAMQMAYRF
NGIGVTQNVLYENQKLIANQFNSAIGKIQDSLSSTASALGKLQDVVNQNAQALNTLVKQLSSNFGAISS
VLNDILSRLDKVEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDLGDISGINASVVNIQKEI
DRLNEVAKNLNESLIDLQELGKYEQYIKWPWYIWLGFIAGLIAIVMVTIMLCCMTSCCSCLKGCCSCGS
*CKFDEDDSEPVLKGVKLHYT*
Nucleotide sequence (showing changes in bold lowercase) (SEQ ID NO: 8):
ATGTTCGTGTTCCTGGTGCTCCTGCCTCTGGTGAGCAGCCAGTGCGTGAACCTGACCACCCGAACC
CAGCTCCCACCAGCCTACACCAACAGCTTTACACGGGGCGTGTACTACCCTGACAAGGTGTTCAGA
TCTAGCGTCCTGCACAGCACTCAGGACCTCTTCCTGCCGTTCTTCAGCAACGTGACAcGGTTCCACG
CCATCCACGTGAGCGGCACAAACGGAACCAAGCGGTTTGATAACCCCGTCCTGCCATTCAATGAT
GGAGTTTACTTCGCCAGTACCGAGAAGAGTAACATCATCCGGGGCTGGATCTTCGGCACCACCCT
GGATAGCAAAACACAGAGCCTCCTGATCGTGAACAATGCCACGAACGTCGTGATCAAGGTGTGCG
AGTTCCAGTTTTGCAATGATCCTTTCCTGGGTGTGTACTACCACAAGAACAACAAGAGCTGGATGG
AAAGCGAGTTCAGAGTCTACAGCAGCGCCAACAACTGCACATTCGAGTACGTCTCTCAGCCTTTTC
TGATGGACCTTGAGGGGAAACAAGGCAACTTCAAGAACCTGAGAGAATTCGTGTTCAAGAACATC
GACGGCTACTTCAAAATCTACTCCAAGCACACACCCATCAACCTGGTCCGGGACCTCCCTCAGGGC
TTCAGCGCCCTGGAACCCCTGGTCGACCTGCCCATAGGCATCAACATAACGCGGTTCCAAACCCTG
CTGGCCCTGCATAGATCCTACCTGACTCCTGGCGACAGCAGCAGCGGATGGACCGCCaGAGCTGC
AGCCTACTATGTGGGCTACCTGCAACCTAGAACCTTCCTGCTGAAGTACAACGAGAACGGCACAAT
CACAGACGCCGTCGACTGCGCCCTGGACCCTCTCTCTGAGACAAAGTGCACCCTGAAGTCCTTCAC
CGTGGAAAAGGGCATCTACCAGACCAGCAACTTCCGGGTGCAGCCTACAGAGAGCATCGTGCGAT
TTCCAAACATTACCAACCTCTGCCCCTTCGGCGAGGTGTTTAACGCCACAAGATTTGCCTCCGTTTA
CGCCTGGAATAGAAAGAGAATCAGCAATTGTGTGGCCGACTACTCCGTGCTGTATAACAGCaCCT
CTTTCAGCACCTTCAAGTGCTACGGCGTTTCCCCAACAAAGCTGAATGACCTGTGCTTCACCAACGT
GTACGCCGACTCCTTCGTAATTAGAGGCGATGAGGTGCGGCAGATCGCACCAGGCCAGACCGGTA
AGATCGCTGACTACAACTATAAGCTGCCTGATGATTTTACAGGCTGCGTGATCGCCTGGAACTCTA
ACAACCTGGATAGCAAGGTGGGCGGCAACTACAACTACCTGTACCGGCTGTTTCGCAAGTCTAAC
CTGAAACCTTTCGAGAGAGACATCTCCACAGAGATCTACCAGGCCGGTTCTACACCTTGTAACGGG
GTGGAAGGCTTCAACTGTTACTTCCCTCTGCAAAGCTACGGCTTCCAGCCTACCAATGGAGTCGGC
TACCAGCCATACCGGGTGGTCGTGCTGTCCTTCGAGTTACTCCACGCCCCCGCCACCGTCTGCGGT
CCTAAGAAGTCCACCAATCTGGTTAAGAACAAATGCGTGAACTTCAACTTCAACGGCCTGACCGG
GACCGGCGTGCTGACCGAAAGCAACAAAAAGTTCCTCCCCTTCCAGCAGTTCGGCCGTGATATCG
CTGACACCACAGATGCCGTCAGAGATCCACAGACCCTGGAAATCCTGGATATTACACCCTGCTCCT
TCGGAGGAGTTTCTGTGATCACCCCCGGGACCAATACCAGCAACCAGGTGGCTGTGCTGTACCAA
GATGTTAACTGCACCGAGGTTCCTGTGGCCATCCACGCCGATCAGCTGACACCTACTTGGAGAGT
GTACTCCACTGGCTCCAATGTGTTCCAGACCAGGGCCGGATGTCTGATCGGCGCCGAGtACGTGA
ATAACAGTTACGAGTGCGACATCCCTATCGGCGCCGGCATCTGTGCCAGCTACCAGACCCAGACA
AACAGCCCTAGACGGGCTgGATCTGTAGCTAGCCAGAGCATCATCGCCTACACCATGAGCCTGGG
CGCAGAGAACAGCGTGGCCTATTCCAACAACTCTATCGCCATTCCCACCAACTTTACAATTAGCGTC
ACAACAGAGATCCTGCCCGTGAGCATGACCAAGACCAGCGTGGACTGTACAATGTACATCTGTGG
CGACAGCACTGAATGCAGCAACCTGCTGCTGCAATACGGCTCCTTTTGCACCCAACTGAACCGGGC
GCTGACCGGAATCGCCGTGGAACAGGACAAAAATACCCAGGAGGTGTTCGCCCAAGTGAAGCAG
ATCTACAAGACCCCACCTATCAAGGACTTCGGCGGCTTTAACTTTAGCCAGATTCTCCCTGATCCTT
CTAAGCCTAGCAAGCGGAGCTTTATCGAGGATCTGCTGTTCAACAAGGTCACCCTGGCCGATGCC
GGCTTTATCAAACAGTATGGCGATTGCCTGGGCGACATAGCCGCCAGAGATCTGATCTGCGCCCA
GAAATTCAACGGCCTGACAGTTCTCCCACCTCTGCTGACCGACGAGATGATCGCTCAGTACACCTC
TGCCCTGCTGGCTGGCACCATCACATCTGGGTGGACATTTGGCGCCGGCGCCGCCCTGCAGATCC
CCTTTGCCATGCAGATGGCCTATAGATTCAACGGAATCGGCGTGACCCAGAACGTGCTGTATGAA
AACCAGAAGCTGATCGCTAACCAGTTCAATTCTGCCATCGGCAAGATCCAGGACTCCCTCTCCTCT
ACCGCCAGCGCCCTGGGCAAACTGCAGGACGTGGTGAATCAGAACGCCCAAGCCCTGAACACCCT
GGTGAAGCAGCTCAGCAGCAATTTTGGCGCCATCAGCTCTGTGCTGAACGATATCCTGTCTAGACT
GGACAAGGTGGAAGCCGAAGTCCAGATCGATAGACTGATCACAGGCAGACTGCAGTCCCTGCAA
ACCTACGTGACCCAACAGCTGATCAGGGCCGCTGAAATAAGAGCCAGCGCCAATCTCGCCGCTAC
CAAGATGTCCGAGTGTGTGCTGGGACAGTCTAAACGCGTTGACTTCTGCGGCAAAGGCTATCACC
TGATGAGCTTCCCCCAGAGCGCGCCGCACGGCGTGGTGTTCCTGCATGTGACATACGTGCCTGCC
CAAGAGAAGAATTTCACAACCGCCCCTGCCATCTGCCACGACGGCAAGGCCCACTTCCCAAGAGA
GGGCGTTTTCGTTTCCAATGGCACACACTGGTTCGTGACACAAAGAAACTTCTACGAACCCCAGAT
TATCACCACCGACAACACCTTCGTGAGTGGCAATTGTGACGTGGTCATCGGAATCGTGAACAACAC
AGTGTACGACCCTCTGCAACCTGAGCTGGACTCTTTTAAGGAAGAGCTGGACAAGTACTTTAAAAA
CCACACCAGCCCCGATGTGGACCTGGGCGACATCAGTGGCATTAACGCCAGCGTGGTGAACATCC
AAAAGGAAATCGACAGACTGAACGAGGTGGCCAAGAACCTGAACGAGTCCCTGATCGACCTGCA
GGAGCTCGGCAAATACGAGCAGTACATCAAGTGGCCTTGGTACATCTGGCTGGGATTCATCGCCG
GACTGATCGCCATCGTGATGGTGACCATCATGCTGTGCTGTATGACCAGCTGCTGCAGTTGCCTGA
AGGGCTGTTGCAGCTGCGGCAGCTGaTGCAAGTTCGACGAAGATGACTCTGAGCCTGTGCTGAAG
GGCGTGAAGCTGCACTACACCTGA
SARS-CoV-2 Spike-Plaque #3
Amino acid sequence (showing changes in bold and underlined)
(SEQ ID NO: 9 and SEQ ID NO: 15):
Mutations: W64R, D253N, G261R, A372T, H655Y and C1253*
MFVFLVLLPLVSSQCVNLTTRTQLPPAYTNSFTRGVYYPDKVFRSSVLHSTQDLFLPFFSNVTRFHAIHV
SGTNGTKRFDNPVLPFNDGVYFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLREFVFKNIDGYFKIYSKHT
PINLVRDLPQGFSALEPLVDLPIGINITRFQTLLALHRSYLTPGNSSSGWTARAAAYYVGYLQPRTFLLKY
NENGTITDAVDCALDPLSETKCTLKSFTVEKGIYQTSNFRVQPTESIVRFPNITNLCPFGEVFNATRFASV
YAWNRKRISNCVADYSVLYNSTSFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGSTPCNGVEGFNC
YFPLQSYGFQPTNGVGYQPYRVVVLSFELLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQDVNCTEVPVAIHA
DQLTPTWRVYSTGSNVFQTRAGCLIGAEYVNNSYECDIPIGAGICASYQTQTNSPRRARSVASQSIIAYT
MSLGAENSVAYSNNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSFIEDLLFNKVTLADAGFIKQYG
DCLGDIAARDLICAQKFNGLTVLPPLLTDEMIAQYTSALLAGTITSGWTFGAGAALQIPFAMQMAYRF
NGIGVTQNVLYENQKLIANQFNSAIGKIQDSLSSTASALGKLQDVVNQNAQALNTLVKQLSSNFGAISS
VLNDILSRLDKVEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDLGDISGINASVVNIQKEI
DRLNEVAKNLNESLIDLQELGKYEQYIKWPWYIWLGFIAGLIAIVMVTIMLCCMTSCCSCLKGCCSCGS
*CKFDEDDSEPVLKGVKLHYT*
Nucleotide sequence (showing changes in bold lowercase) (SEQ ID NO: 10):
ATGTTCGTGTTCCTGGTGCTCCTGCCTCTGGTGAGCAGCCAGTGCGTGAACCTGACCACCCGAACC
CAGCTCCCACCAGCCTACACCAACAGCTTTACACGGGGCGTGTACTACCCTGACAAGGTGTTCAGA
TCTAGCGTCCTGCACAGCACTCAGGACCTCTTCCTGCCGTTCTTCAGCAACGTGACAcGGTTCCACG
CCATCCACGTGAGCGGCACAAACGGAACCAAGCGGTTTGATAACCCCGTCCTGCCATTCAATGAT
GGAGTTTACTTCGCCAGTACCGAGAAGAGTAACATCATCCGGGGCTGGATCTTCGGCACCACCCT
GGATAGCAAAACACAGAGCCTCCTGATCGTGAACAATGCCACGAACGTCGTGATCAAGGTGTGCG
AGTTCCAGTTTTGCAATGATCCTTTCCTGGGTGTGTACTACCACAAGAACAACAAGAGCTGGATGG
AAAGCGAGTTCAGAGTCTACAGCAGCGCCAACAACTGCACATTCGAGTACGTCTCTCAGCCTTTTC
TGATGGACCTTGAGGGGAAACAAGGCAACTTCAAGAACCTGAGAGAATTCGTGTTCAAGAACATC
GACGGCTACTTCAAAATCTACTCCAAGCACACACCCATCAACCTGGTCCGGGACCTCCCTCAGGGC
TTCAGCGCCCTGGAACCCCTGGTCGACCTGCCCATAGGCATCAACATAACGCGGTTCCAAACCCTG
CTGGCCCTGCATAGATCCTACCTGACTCCTGGCaACAGCAGCAGCGGATGGACCGCCaGAGCTGC
AGCCTACTATGTGGGCTACCTGCAACCTAGAACCTTCCTGCTGAAGTACAACGAGAACGGCACAAT
CACAGACGCCGTCGACTGCGCCCTGGACCCTCTCTCTGAGACAAAGTGCACCCTGAAGTCCTTCAC
CGTGGAAAAGGGCATCTACCAGACCAGCAACTTCCGGGTGCAGCCTACAGAGAGCATCGTGCGAT
TTCCAAACATTACCAACCTCTGCCCCTTCGGCGAGGTGTTTAACGCCACAAGATTTGCCTCCGTTTA
CGCCTGGAATAGAAAGAGAATCAGCAATTGTGTGGCCGACTACTCCGTGCTGTATAACAGCaCCT
CTTTCAGCACCTTCAAGTGCTACGGCGTTTCCCCAACAAAGCTGAATGACCTGTGCTTCACCAACGT
GTACGCCGACTCCTTCGTAATTAGAGGCGATGAGGTGCGGCAGATCGCACCAGGCCAGACCGGTA
AGATCGCTGACTACAACTATAAGCTGCCTGATGATTTTACAGGCTGCGTGATCGCCTGGAACTCTA
ACAACCTGGATAGCAAGGTGGGCGGCAACTACAACTACCTGTACCGGCTGTTTCGCAAGTCTAAC
CTGAAACCTTTCGAGAGAGACATCTCCACAGAGATCTACCAGGCCGGTTCTACACCTTGTAACGGG
GTGGAAGGCTTCAACTGTTACTTCCCTCTGCAAAGCTACGGCTTCCAGCCTACCAATGGAGTCGGC
TACCAGCCATACCGGGTGGTCGTGCTGTCCTTCGAGTTACTCCACGCCCCCGCCACCGTCTGCGGT
CCTAAGAAGTCCACCAATCTGGTTAAGAACAAATGCGTGAACTTCAACTTCAACGGCCTGACCGG
GACCGGCGTGCTGACCGAAAGCAACAAAAAGTTCCTCCCCTTCCAGCAGTTCGGCCGTGATATCG
CTGACACCACAGATGCCGTCAGAGATCCACAGACCCTGGAAATCCTGGATATTACACCCTGCTCCT
TCGGAGGAGTTTCTGTGATCACCCCCGGGACCAATACCAGCAACCAGGTGGCTGTGCTGTACCAA
GATGTTAACTGCACCGAGGTTCCTGTGGCCATCCACGCCGATCAGCTGACACCTACTTGGAGAGT
GTACTCCACTGGCTCCAATGTGTTCCAGACCAGGGCCGGATGTCTGATCGGCGCCGAGtACGTGA
ATAACAGTTACGAGTGCGACATCCCTATCGGCGCCGGCATCTGTGCCAGCTACCAGACCCAGACA
AACAGCCCTAGACGGGCTAGATCTGTAGCTAGCCAGAGCATCATCGCCTACACCATGAGCCTGGG
CGCAGAGAACAGCGTGGCCTATTCCAACAACTCTATCGCCATTCCCACCAACTTTACAATTAGCGTC
ACAACAGAGATCCTGCCCGTGAGCATGACCAAGACCAGCGTGGACTGTACAATGTACATCTGTGG
CGACAGCACTGAATGCAGCAACCTGCTGCTGCAATACGGCTCCTTTTGCACCCAACTGAACCGGGC
GCTGACCGGAATCGCCGTGGAACAGGACAAAAATACCCAGGAGGTGTTCGCCCAAGTGAAGCAG
ATCTACAAGACCCCACCTATCAAGGACTTCGGCGGCTTTAACTTTAGCCAGATTCTCCCTGATCCTT
CTAAGCCTAGCAAGCGGAGCTTTATCGAGGATCTGCTGTTCAACAAGGTCACCCTGGCCGATGCC
GGCTTTATCAAACAGTATGGCGATTGCCTGGGCGACATAGCCGCCAGAGATCTGATCTGCGCCCA
GAAATTCAACGGCCTGACAGTTCTCCCACCTCTGCTGACCGACGAGATGATCGCTCAGTACACCTC
TGCCCTGCTGGCTGGCACCATCACATCTGGGTGGACATTTGGCGCCGGCGCCGCCCTGCAGATCC
CCTTTGCCATGCAGATGGCCTATAGATTCAACGGAATCGGCGTGACCCAGAACGTGCTGTATGAA
AACCAGAAGCTGATCGCTAACCAGTTCAATTCTGCCATCGGCAAGATCCAGGACTCCCTCTCCTCT
ACCGCCAGCGCCCTGGGCAAACTGCAGGACGTGGTGAATCAGAACGCCCAAGCCCTGAACACCCT
GGTGAAGCAGCTCAGCAGCAATTTTGGCGCCATCAGCTCTGTGCTGAACGATATCCTGTCTAGACT
GGACAAGGTGGAAGCCGAAGTCCAGATCGATAGACTGATCACAGGCAGACTGCAGTCCCTGCAA
ACCTACGTGACCCAACAGCTGATCAGGGCCGCTGAAATAAGAGCCAGCGCCAATCTCGCCGCTAC
CAAGATGTCCGAGTGTGTGCTGGGACAGTCTAAACGCGTTGACTTCTGCGGCAAAGGCTATCACC
TGATGAGCTTCCCCCAGAGCGCGCCGCACGGCGTGGTGTTCCTGCATGTGACATACGTGCCTGCC
CAAGAGAAGAATTTCACAACCGCCCCTGCCATCTGCCACGACGGCAAGGCCCACTTCCCAAGAGA
GGGCGTTTTCGTTTCCAATGGCACACACTGGTTCGTGACACAAAGAAACTTCTACGAACCCCAGAT
TATCACCACCGACAACACCTTCGTGAGTGGCAATTGTGACGTGGTCATCGGAATCGTGAACAACAC
AGTGTACGACCCTCTGCAACCTGAGCTGGACTCTTTTAAGGAAGAGCTGGACAAGTACTTTAAAAA
CCACACCAGCCCCGATGTGGACCTGGGCGACATCAGTGGCATTAACGCCAGCGTGGTGAACATCC
AAAAGGAAATCGACAGACTGAACGAGGTGGCCAAGAACCTGAACGAGTCCCTGATCGACCTGCA
GGAGCTCGGCAAATACGAGCAGTACATCAAGTGGCCTTGGTACATCTGGCTGGGATTCATCGCCG
GACTGATCGCCATCGTGATGGTGACCATCATGCTGTGCTGTATGACCAGCTGCTGCAGTTGCCTGA
AGGGCTGTTGCAGCTGCGGCAGCTGaTGCAAGTTCGACGAAGATGACTCTGAGCCTGTGCTGAAG
GGCGTGAAGCTGCACTACACCTGA
SARS-CoV-2 Spike-Plaque #4 and #5
Amino acid sequence (showing changes in bold and underlined)
(SEQ ID NO: 11 and SEQ ID NO: 15):
Mutations: W64R, D253N, G261R, A372T, H655Y, R685G and C1253*
MFVFLVLLPLVSSQCVNLTTRTQLPPAYTNSFTRGVYYPDKVFRSSVLHSTQDLFLPFFSNVTRFHAIHV
SGTNGTKRFDNPVLPFNDGVYFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLREFVFKNIDGYFKIYSKHT
PINLVRDLPQGFSALEPLVDLPIGINITRFQTLLALHRSYLTPGNSSSGWTARAAAYYVGYLQPRTFLLKY
NENGTITDAVDCALDPLSETKCTLKSFTVEKGIYQTSNFRVQPTESIVRFPNITNLCPFGEVFNATRFASV
YAWNRKRISNCVADYSVLYNSTSFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGSTPCNGVEGFNC
YFPLQSYGFQPTNGVGYQPYRVVVLSFELLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQDVNCTEVPVAIHA
DQLTPTWRVYSTGSNVFQTRAGCLIGAEYVNNSYECDIPIGAGICASYQTQTNSPRRAGSVASQSIIAYT
MSLGAENSVAYSNNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSFIEDLLFNKVTLADAGFIKQYG
DCLGDIAARDLICAQKFNGLTVLPPLLTDEMIAQYTSALLAGTITSGWTFGAGAALQIPFAMQMAYRF
NGIGVTQNVLYENQKLIANQFNSAIGKIQDSLSSTASALGKLQDVVNQNAQALNTLVKQLSSNFGAISS
VLNDILSRLDKVEAEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDLGDISGINASVVNIQKEI
DRLNEVAKNLNESLIDLQELGKYEQYIKWPWYIWLGFIAGLIAIVMVTIMLCCMTSCCSCLKGCCSCGS
*CKFDEDDSEPVLKGVKLHYT*
Nucleotide sequence (showing changes in bold lowercase) (SEQ ID NO: 12):
ATGTTCGTGTTCCTGGTGCTCCTGCCTCTGGTGAGCAGCCAGTGCGTGAACCTGACCACCCGAACC
CAGCTCCCACCAGCCTACACCAACAGCTTTACACGGGGCGTGTACTACCCTGACAAGGTGTTCAGA
TCTAGCGTCCTGCACAGCACTCAGGACCTCTTCCTGCCGTTCTTCAGCAACGTGACACGGTTCCACG
CCATCCACGTGAGCGGCACAAACGGAACCAAGCGGTTTGATAACCCCGTCCTGCCATTCAATGAT
GGAGTTTACTTCGCCAGTACCGAGAAGAGTAACATCATCCGGGGCTGGATCTTCGGCACCACCCT
GGATAGCAAAACACAGAGCCTCCTGATCGTGAACAATGCCACGAACGTCGTGATCAAGGTGTGCG
AGTTCCAGTTTTGCAATGATCCTTTCCTGGGTGTGTACTACCACAAGAACAACAAGAGCTGGATGG
AAAGCGAGTTCAGAGTCTACAGCAGCGCCAACAACTGCACATTCGAGTACGTCTCTCAGCCTTTTC
TGATGGACCTTGAGGGGAAACAAGGCAACTTCAAGAACCTGAGAGAATTCGTGTTCAAGAACATC
GACGGCTACTTCAAAATCTACTCCAAGCACACACCCATCAACCTGGTCCGGGACCTCCCTCAGGGC
TTCAGCGCCCTGGAACCCCTGGTCGACCTGCCCATAGGCATCAACATAACGCGGTTCCAAACCCTG
CTGGCCCTGCATAGATCCTACCTGACTCCTGGCaACAGCAGCAGCGGATGGACCGCCaGAGCTGC
AGCCTACTATGTGGGCTACCTGCAACCTAGAACCTTCCTGCTGAAGTACAACGAGAACGGCACAAT
CACAGACGCCGTCGACTGCGCCCTGGACCCTCTCTCTGAGACAAAGTGCACCCTGAAGTCCTTCAC
CGTGGAAAAGGGCATCTACCAGACCAGCAACTTCCGGGTGCAGCCTACAGAGAGCATCGTGCGAT
TTCCAAACATTACCAACCTCTGCCCCTTCGGCGAGGTGTTTAACGCCACAAGATTTGCCTCCGTTTA
CGCCTGGAATAGAAAGAGAATCAGCAATTGTGTGGCCGACTACTCCGTGCTGTATAACAGCaCCT
CTTTCAGCACCTTCAAGTGCTACGGCGTTTCCCCAACAAAGCTGAATGACCTGTGCTTCACCAACGT
GTACGCCGACTCCTTCGTAATTAGAGGCGATGAGGTGCGGCAGATCGCACCAGGCCAGACCGGTA
AGATCGCTGACTACAACTATAAGCTGCCTGATGATTTTACAGGCTGCGTGATCGCCTGGAACTCTA
ACAACCTGGATAGCAAGGTGGGCGGCAACTACAACTACCTGTACCGGCTGTTTCGCAAGTCTAAC
CTGAAACCTTTCGAGAGAGACATCTCCACAGAGATCTACCAGGCCGGTTCTACACCTTGTAACGGG
GTGGAAGGCTTCAACTGTTACTTCCCTCTGCAAAGCTACGGCTTCCAGCCTACCAATGGAGTCGGC
TACCAGCCATACCGGGTGGTCGTGCTGTCCTTCGAGTTACTCCACGCCCCCGCCACCGTCTGCGGT
CCTAAGAAGTCCACCAATCTGGTTAAGAACAAATGCGTGAACTTCAACTTCAACGGCCTGACCGG
GACCGGCGTGCTGACCGAAAGCAACAAAAAGTTCCTCCCCTTCCAGCAGTTCGGCCGTGATATCG
CTGACACCACAGATGCCGTCAGAGATCCACAGACCCTGGAAATCCTGGATATTACACCCTGCTCCT
TCGGAGGAGTTTCTGTGATCACCCCCGGGACCAATACCAGCAACCAGGTGGCTGTGCTGTACCAA
GATGTTAACTGCACCGAGGTTCCTGTGGCCATCCACGCCGATCAGCTGACACCTACTTGGAGAGT
GTACTCCACTGGCTCCAATGTGTTCCAGACCAGGGCCGGATGTCTGATCGGCGCCGAGtACGTGA
ATAACAGTTACGAGTGCGACATCCCTATCGGCGCCGGCATCTGTGCCAGCTACCAGACCCAGACA
AACAGCCCTAGACGGGCTgGATCTGTAGCTAGCCAGAGCATCATCGCCTACACCATGAGCCTGGG
CGCAGAGAACAGCGTGGCCTATTCCAACAACTCTATCGCCATTCCCACCAACTTTACAATTAGCGTC
ACAACAGAGATCCTGCCCGTGAGCATGACCAAGACCAGCGTGGACTGTACAATGTACATCTGTGG
CGACAGCACTGAATGCAGCAACCTGCTGCTGCAATACGGCTCCTTTTGCACCCAACTGAACCGGGC
GCTGACCGGAATCGCCGTGGAACAGGACAAAAATACCCAGGAGGTGTTCGCCCAAGTGAAGCAG
ATCTACAAGACCCCACCTATCAAGGACTTCGGCGGCTTTAACTTTAGCCAGATTCTCCCTGATCCTT
CTAAGCCTAGCAAGCGGAGCTTTATCGAGGATCTGCTGTTCAACAAGGTCACCCTGGCCGATGCC
GGCTTTATCAAACAGTATGGCGATTGCCTGGGCGACATAGCCGCCAGAGATCTGATCTGCGCCCA
GAAATTCAACGGCCTGACAGTTCTCCCACCTCTGCTGACCGACGAGATGATCGCTCAGTACACCTC
TGCCCTGCTGGCTGGCACCATCACATCTGGGTGGACATTTGGCGCCGGCGCCGCCCTGCAGATCC
CCTTTGCCATGCAGATGGCCTATAGATTCAACGGAATCGGCGTGACCCAGAACGTGCTGTATGAA
AACCAGAAGCTGATCGCTAACCAGTTCAATTCTGCCATCGGCAAGATCCAGGACTCCCTCTCCTCT
ACCGCCAGCGCCCTGGGCAAACTGCAGGACGTGGTGAATCAGAACGCCCAAGCCCTGAACACCCT
GGTGAAGCAGCTCAGCAGCAATTTTGGCGCCATCAGCTCTGTGCTGAACGATATCCTGTCTAGACT
GGACAAGGTGGAAGCCGAAGTCCAGATCGATAGACTGATCACAGGCAGACTGCAGTCCCTGCAA
ACCTACGTGACCCAACAGCTGATCAGGGCCGCTGAAATAAGAGCCAGCGCCAATCTCGCCGCTAC
CAAGATGTCCGAGTGTGTGCTGGGACAGTCTAAACGCGTTGACTTCTGCGGCAAAGGCTATCACC
TGATGAGCTTCCCCCAGAGCGCGCCGCACGGCGTGGTGTTCCTGCATGTGACATACGTGCCTGCC
CAAGAGAAGAATTTCACAACCGCCCCTGCCATCTGCCACGACGGCAAGGCCCACTTCCCAAGAGA
GGGCGTTTTCGTTTCCAATGGCACACACTGGTTCGTGACACAAAGAAACTTCTACGAACCCCAGAT
TATCACCACCGACAACACCTTCGTGAGTGGCAATTGTGACGTGGTCATCGGAATCGTGAACAACAC
AGTGTACGACCCTCTGCAACCTGAGCTGGACTCTTTTAAGGAAGAGCTGGACAAGTACTTTAAAAA
CCACACCAGCCCCGATGTGGACCTGGGCGACATCAGTGGCATTAACGCCAGCGTGGTGAACATCC
AAAAGGAAATCGACAGACTGAACGAGGTGGCCAAGAACCTGAACGAGTCCCTGATCGACCTGCA
GGAGCTCGGCAAATACGAGCAGTACATCAAGTGGCCTTGGTACATCTGGCTGGGATTCATCGCCG
GACTGATCGCCATCGTGATGGTGACCATCATGCTGTGCTGTATGACCAGCTGCTGCAGTTGCCTGA
AGGGCTGTTGCAGCTGCGGCAGCTGaTGCAAGTTCGACGAAGATGACTCTGAGCCTGTGCTGAAG
GGCGTGAAGCTGCACTACACCTGA
SARS-CoV-2 Spike-Plaque #6
Amino acid sequence (showing changes in bold and underlined)
(SEQ ID NO: 13 and SEQ ID NO: 15):
Mutations: W64R, G261R, A372T, R685G and C1253*
MFVFLVLLPLVSSQCVNLTTRTQLPPAYTNSFTRGVYYPDKVFRSSVLHSTQDLFLPFFSNVTRFHAIHV
SGTNGTKRFDNPVLPFNDGVYFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLREFVFKNIDGYFKIYSKHT
PINLVRDLPQGFSALEPLVDLPIGINITRFQTLLALHRSYLTPGDSSSGWTARAAAYYVGYLQPRTFLLKY
NENGTITDAVDCALDPLSETKCTLKSFTVEKGIYQTSNFRVQPTESIVRFPNITNLCPFGEVFNATRFASV
YAWNRKRISNCVADYSVLYNSTSFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGSTPCNGVEGFNC
YFPLQSYGFQPTNGVGYQPYRVVVLSFELLHAPATVCGPKKSTNLVKNKCVNFNFNGLTGTGVLTESN
KKFLPFQQFGRDIADTTDAVRDPQTLEILDITPCSFGGVSVITPGTNTSNQVAVLYQDVNCTEVPVAIHA
DQLTPTWRVYSTGSNVFQTRAGCLIGAEHVNNSYECDIPIGAGICASYQTQTNSPRRAGSVASQSIIAY
TMSLGAENSVAYSNNSIAIPTNFTISVTTEILPVSMTKTSVDCTMYICGDSTECSNLLLQYGSFCTQLNRA
LTGIAVEQDKNTQEVFAQVKQIYKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNQNAQALNTLVKQLSSNFGAIS
SVLNDILSRLDKVEAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGTHWFVTQRNFYE
PQIITTDNTFVSGNCDVVIGIVNNTVYDPLQPELDSFKEELDKYFKNHTSPDVDLGDISGINASVVNIQKE
IDRLNEVAKNLNESLIDLQELGKYEQYIKWPWYIWLGFIAGLIAIVMVTIMLCCMTSCCSCLKGCCSCGS
*CKFDEDDSEPVLKGVKLHYT*
Nucleotide sequence (showing changes in bold lowercase) (SEQ ID NO: 14):
ATGTTCGTGTTCCTGGTGCTCCTGCCTCTGGTGAGCAGCCAGTGCGTGAACCTGACCACCCGAACC
CAGCTCCCACCAGCCTACACCAACAGCTTTACACGGGGCGTGTACTACCCTGACAAGGTGTTCAGA
TCTAGCGTCCTGCACAGCACTCAGGACCTCTTCCTGCCGTTCTTCAGCAACGTGACAcGGTTCCACG
CCATCCACGTGAGCGGCACAAACGGAACCAAGCGGTTTGATAACCCCGTCCTGCCATTCAATGAT
GGAGTTTACTTCGCCAGTACCGAGAAGAGTAACATCATCCGGGGCTGGATCTTCGGCACCACCCT
GGATAGCAAAACACAGAGCCTCCTGATCGTGAACAATGCCACGAACGTCGTGATCAAGGTGTGCG
AGTTCCAGTTTTGCAATGATCCTTTCCTGGGTGTGTACTACCACAAGAACAACAAGAGCTGGATGG
AAAGCGAGTTCAGAGTCTACAGCAGCGCCAACAACTGCACATTCGAGTACGTCTCTCAGCCTTTTC
TGATGGACCTTGAGGGGAAACAAGGCAACTTCAAGAACCTGAGAGAATTCGTGTTCAAGAACATC
GACGGCTACTTCAAAATCTACTCCAAGCACACACCCATCAACCTGGTCCGGGACCTCCCTCAGGGC
TTCAGCGCCCTGGAACCCCTGGTCGACCTGCCCATAGGCATCAACATAACGCGGTTCCAAACCCTG
CTGGCCCTGCATAGATCCTACCTGACTCCTGGCGACAGCAGCAGCGGATGGACCGCCaGAGCTGC
AGCCTACTATGTGGGCTACCTGCAACCTAGAACCTTCCTGCTGAAGTACAACGAGAACGGCACAAT
CACAGACGCCGTCGACTGCGCCCTGGACCCTCTCTCTGAGACAAAGTGCACCCTGAAGTCCTTCAC
CGTGGAAAAGGGCATCTACCAGACCAGCAACTTCCGGGTGCAGCCTACAGAGAGCATCGTGCGAT
TTCCAAACATTACCAACCTCTGCCCCTTCGGCGAGGTGTTTAACGCCACAAGATTTGCCTCCGTTTA
CGCCTGGAATAGAAAGAGAATCAGCAATTGTGTGGCCGACTACTCCGTGCTGTATAACAGCaCCT
CTTTCAGCACCTTCAAGTGCTACGGCGTTTCCCCAACAAAGCTGAATGACCTGTGCTTCACCAACGT
GTACGCCGACTCCTTCGTAATTAGAGGCGATGAGGTGCGGCAGATCGCACCAGGCCAGACCGGTA
AGATCGCTGACTACAACTATAAGCTGCCTGATGATTTTACAGGCTGCGTGATCGCCTGGAACTCTA
ACAACCTGGATAGCAAGGTGGGCGGCAACTACAACTACCTGTACCGGCTGTTTCGCAAGTCTAAC
CTGAAACCTTTCGAGAGAGACATCTCCACAGAGATCTACCAGGCCGGTTCTACACCTTGTAACGGG
GTGGAAGGCTTCAACTGTTACTTCCCTCTGCAAAGCTACGGCTTCCAGCCTACCAATGGAGTCGGC
TACCAGCCATACCGGGTGGTCGTGCTGTCCTTCGAGTTACTCCACGCCCCCGCCACCGTCTGCGGT
CCTAAGAAGTCCACCAATCTGGTTAAGAACAAATGCGTGAACTTCAACTTCAACGGCCTGACCGG
GACCGGCGTGCTGACCGAAAGCAACAAAAAGTTCCTCCCCTTCCAGCAGTTCGGCCGTGATATCG
CTGACACCACAGATGCCGTCAGAGATCCACAGACCCTGGAAATCCTGGATATTACACCCTGCTCCT
TCGGAGGAGTTTCTGTGATCACCCCCGGGACCAATACCAGCAACCAGGTGGCTGTGCTGTACCAA
GATGTTAACTGCACCGAGGTTCCTGTGGCCATCCACGCCGATCAGCTGACACCTACTTGGAGAGT
GTACTCCACTGGCTCCAATGTGTTCCAGACCAGGGCCGGATGTCTGATCGGCGCCGAGCACGTGA
ATAACAGTTACGAGTGCGACATCCCTATCGGCGCCGGCATCTGTGCCAGCTACCAGACCCAGACA
AACAGCCCTAGACGGGCTgGATCTGTAGCTAGCCAGAGCATCATCGCCTACACCATGAGCCTGGG
CGCAGAGAACAGCGTGGCCTATTCCAACAACTCTATCGCCATTCCCACCAACTTTACAATTAGCGTC
ACAACAGAGATCCTGCCCGTGAGCATGACCAAGACCAGCGTGGACTGTACAATGTACATCTGTGG
CGACAGCACTGAATGCAGCAACCTGCTGCTGCAATACGGCTCCTTTTGCACCCAACTGAACCGGGC
GCTGACCGGAATCGCCGTGGAACAGGACAAAAATACCCAGGAGGTGTTCGCCCAAGTGAAGCAG
ATCTACAAGACCCCACCTATCAAGGACTTCGGCGGCTTTAACTTTAGCCAGATTCTCCCTGATCCTT
CTAAGCCTAGCAAGCGGAGCTTTATCGAGGATCTGCTGTTCAACAAGGTCACCCTGGCCGATGCC
GGCTTTATCAAACAGTATGGCGATTGCCTGGGCGACATAGCCGCCAGAGATCTGATCTGCGCCCA
GAAATTCAACGGCCTGACAGTTCTCCCACCTCTGCTGACCGACGAGATGATCGCTCAGTACACCTC
TGCCCTGCTGGCTGGCACCATCACATCTGGGTGGACATTTGGCGCCGGCGCCGCCCTGCAGATCC
CCTTTGCCATGCAGATGGCCTATAGATTCAACGGAATCGGCGTGACCCAGAACGTGCTGTATGAA
AACCAGAAGCTGATCGCTAACCAGTTCAATTCTGCCATCGGCAAGATCCAGGACTCCCTCTCCTCT
ACCGCCAGCGCCCTGGGCAAACTGCAGGACGTGGTGAATCAGAACGCCCAAGCCCTGAACACCCT
GGTGAAGCAGCTCAGCAGCAATTTTGGCGCCATCAGCTCTGTGCTGAACGATATCCTGTCTAGACT
GGACAAGGTGGAAGCCGAAGTCCAGATCGATAGACTGATCACAGGCAGACTGCAGTCCCTGCAA
ACCTACGTGACCCAACAGCTGATCAGGGCCGCTGAAATAAGAGCCAGCGCCAATCTCGCCGCTAC
CAAGATGTCCGAGTGTGTGCTGGGACAGTCTAAACGCGTTGACTTCTGCGGCAAAGGCTATCACC
TGATGAGCTTCCCCCAGAGCGCGCCGCACGGCGTGGTGTTCCTGCATGTGACATACGTGCCTGCC
CAAGAGAAGAATTTCACAACCGCCCCTGCCATCTGCCACGACGGCAAGGCCCACTTCCCAAGAGA
GGGCGTTTTCGTTTCCAATGGCACACACTGGTTCGTGACACAAAGAAACTTCTACGAACCCCAGAT
TATCACCACCGACAACACCTTCGTGAGTGGCAATTGTGACGTGGTCATCGGAATCGTGAACAACAC
AGTGTACGACCCTCTGCAACCTGAGCTGGACTCTTTTAAGGAAGAGCTGGACAAGTACTTTAAAAA
CCACACCAGCCCCGATGTGGACCTGGGCGACATCAGTGGCATTAACGCCAGCGTGGTGAACATCC
AAAAGGAAATCGACAGACTGAACGAGGTGGCCAAGAACCTGAACGAGTCCCTGATCGACCTGCA
GGAGCTCGGCAAATACGAGCAGTACATCAAGTGGCCTTGGTACATCTGGCTGGGATTCATCGCCG
GACTGATCGCCATCGTGATGGTGACCATCATGCTGTGCTGTATGACCAGCTGCTGCAGTTGCCTGA
AGGGCTGTTGCAGCTGCGGCAGCTGaTGCAAGTTCGACGAAGATGACTCTGAGCCTGTGCTGAAG
GGCGTGAAGCTGCACTACACCTGA
*indicates the stop codon or nonsense mutation. Included in Table 1
are exemplary wild-type and mutated spike glycoprotein S of SARS-CoV-2
comprising a nucleotide sequence or an amino acid sequence having at
least 80%, 81%, 82%, 83%, 84%, 85%, 86%, 87%, 88%, 89%, 90%, 91%, 92%,
93%, 94%, 95%, 96%, 97%, 98%, 99%, 99.5%, or more identity across
their full length with a nucleotide sequence or an amino acid sequence
of any SEQ ID NO listed in Table 1, or a portion thereof. Such spike
glycoprotein S may have a function of the full-length spike
glycoprotein S as described further herein.

In some embodiments, the recombinant VSV vector comprises a nucleic acid encoding a wild-type spike glycoprotein S of SARS-CoV-2 (e.g., the spike glycoprotein S having an amino acid sequence set forth in SEQ ID No: 1), or biologically active fragment thereof. The wild-type spike glycoprotein S of SARS-CoV-2 may have an amino acid sequence set forth in SEQ ID NO: 1, or may be any other naturally evolved variants isolated from nature or infected subjects. In some embodiments, the nucleic acid comprises a nucleotide sequence set forth in SEQ ID No: 2.

In some embodiment, the recombinant VSV vector comprises a nucleic acid encoding a mutated spike glycoprotein S of SARS-CoV-2, or biologically active fragment thereof. The mutated spike glycoprotein S may comprise one or more mutations at positions corresponding to C1250, C1253, L517, P812, W64, D253, G261, A372, H655, and/or R685 of SEQ ID NO: 1. In one embodiment the mutated spike glycoprotein S of SARS-CoV-2 comprises one or more mutations selected from C1250*, C1253*, L517S, P812R, W64R, D253N, G261R, A372T, H655Y, and R685G.

For example, in one embodiment, the mutated spike glycoprotein S of SARS-CoV-2 may comprise mutations A372T, L517S, P812R, C1250* and C1253*. In another embodiment, the mutated spike glycoprotein S of SARS-CoV-2 may comprise mutations W64R, G261R, A372T, L517S, P812R, C1250* and C1253*. The mutated spike glycoprotein S of SARS-CoV-2 may comprise mutations C1253*, W64R, G261R, and A372T. In another embodiment, the mutated spike glycoprotein S of SARS-CoV-2 may further comprise H655Y and/or R685G. For example, in one embodiments, the mutated spike glycoprotein S of SARS-CoV-2 may comprise mutations W64R, G261R, A372T, H655Y, R685G and C1253*. In another embodiment, the mutated spike glycoprotein S of SARS-CoV-2 may comprise mutations W64R, D253N, G261R, A372T, H655Y and C1253*. In still another embodiment, the mutated spike glycoprotein S of SARS-CoV-2 may comprise mutations W64R, D253N, G261R, A372T, H655Y, R685G and C1253*. In yet another embodiment, the mutated spike glycoprotein S of SARS-CoV-2 may comprise mutations W64R, G261R, A372T, R685G and C1253*.

In some embodiments, the mutations of the spike glycoprotein S are capable of facilitating replication of rVSV-SARS-CoV-2 S virus. In some embodiments, the mutations of the spike glycoprotein S are capable of promoting efficient spreading of rVSV-SARS-CoV-2 S virus in in tissue culture with little or no syncytium formation.

In some embodiments, the recombinant VSV vector comprises a nucleic acid encoding a spike glycoprotein S of SARS-CoV-2 comprising an amino acid sequence set forth in SEQ ID NOs: 1, 3, 5, 7, 9, 11, or 13. In some embodiments, the recombinant VSV vector comprises a nucleic acid encoding a spike glycoprotein S of SARS-CoV-2 comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or 100%) identify with the amino acid sequence set forth in SEQ ID NOs: 1, 3, 5, 7, 9, 11, or 13.

In some embodiments, the recombinant VSV vector comprises a nucleic acid comprising a nucleotide sequence set forth in SEQ ID NOs: 2, 4, 6, 8, 10, 12, or 14. In some embodiments, the recombinant VSV vector comprises a nucleic acid comprising a nucleotide sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or 100%) identify with the nucleotide sequence set forth in SEQ ID NOs: 2, 4, 6, 8, 10, 12, or 14.

In some embodiments, the recombinant VSV vector lacks native VSV entry glycoprotein gene, G. In certain embodiments, the native VSV entry glycoprotein gene, G, is replaced with the nucleic acid encoding spike glycoprotein S of SARS-CoV-2.

The recombinant VSV vector may further comprises a nucleic acid encoding a detectable label (e.g., a reporter). In some embodiments, the reporter is a fluorescent protein, including but are not limited to, for example, an enhanced green fluorescent protein (eGFP), GFP, a blue fluorescent protein (BFP), a yellow fluorescent protein (YFP), a red fluorescent protein (RFP), an orange fluorescent protein (OFP), etc.

The recombinant VSV vector disclosed herein can be engineered and modified using standard molecular cloning techniques well known in the art. The recombinant VSV vector may comprise additional modifications (e.g., chemical modifications) that facilitate the stability and delivery into cells.

The present invention also provides the recombinant vesicular stomatitis virus (VSV) generated from the engineered recombinant VSV vector disclosed herein. Accordingly, the present invention provides a recombinant vesicular stomatitis virus (VSV) comprising a spike glycoprotein S of severe acute respiratory syndrome coronavirus-2 (SARS-CoV-2), or biologically active fragment thereof. The present invention also provides a recombinant vesicular stomatitis virus (VSV) comprising a genome which comprises a nucleic acid encoding spike glycoprotein S of severe acute respiratory syndrome coronavirus-2 (SARS-CoV-2), or biologically active fragment thereof. The exemplary nucleotide sequences and amino acid sequences of the spike glycoprotein S encoded by the recombinant VSV of the present invention are shown in Table 1 above.

In some embodiments, the recombinant VSV comprises a wild-type spike glycoprotein S of SARS-CoV-2 (e.g., the spike glycoprotein S having an amino acid sequence set forth in SEQ ID No: 1), or biologically active fragment thereof. In some embodiments, the recombinant VSV comprises a genome comprising a nucleic acid encoding a wild-type spike glycoprotein S of SARS-CoV-2 (e.g., the spike glycoprotein S having an amino acid sequence set forth in SEQ ID No: 1), or biologically active fragment thereof. The wild-type spike glycoprotein S of SARS-CoV-2 may have an amino acid sequence set forth in SEQ ID NO: 1, or may be any other naturally evolved variants isolated from nature or infected subjects. In some embodiments, the genome comprising a nucleic acid comprising a nucleotide sequence set forth in SEQ ID No: 2.

In some embodiment, the recombinant VSV comprises a mutated spike glycoprotein S of SARS-CoV-2, or biologically active fragment thereof. In some embodiment, the recombinant VSV comprises a genome comprising a nucleic acid encoding a mutated spike glycoprotein S of SARS-CoV-2, or biologically active fragment thereof. The mutated spike glycoprotein S may comprise one or more mutations at positions corresponding to C1250, C1253, L517, P812, W64, D253, G261, A372, H655, and/or R685 of SEQ ID NO: 1. In one embodiment the mutated spike glycoprotein S of SARS-CoV-2 comprises one or more mutations selected from C1250*, C1253*, L517S, P812R, W64R, D253N, G261R, A372T, H655Y, and R685G.

For example, in one embodiment, the mutated spike glycoprotein S of SARS-CoV-2 may comprise mutations A372T, L517S, P812R, C1250* and C1253*. In another embodiment, the mutated spike glycoprotein S of SARS-CoV-2 may comprise mutations W64R, G261R, A372T, L517S, P812R, C1250* and C1253*. The mutated spike glycoprotein S of SARS-CoV-2 may comprise mutations C1253*, W64R, G261R, and A372T. In another embodiment, the mutated spike glycoprotein S of SARS-CoV-2 may further comprise H655Y and/or R685G. For example, in one embodiments, the mutated spike glycoprotein S of SARS-CoV-2 may comprise mutations W64R, G261R, A372T, H655Y, R685G and C1253*. In another embodiment, the mutated spike glycoprotein S of SARS-CoV-2 may comprise mutations W64R, D253N, G261R, A372T, H655Y and C1253*. In still another embodiment, the mutated spike glycoprotein S of SARS-CoV-2 may comprise mutations W64R, D253N, G261R, A372T, H655Y, R685G and C1253*. In yet another embodiment, the mutated spike glycoprotein S of SARS-CoV-2 may comprise mutations W64R, G261R, A372T, R685G and C1253*.

In some embodiments, the mutations of the spike glycoprotein S are capable of facilitating replication of rVSV-SARS-CoV-2 S virus. In some embodiments, the mutations of the spike glycoprotein S are capable of promoting efficient spreading of rVSV-SARS-CoV-2 S virus in in tissue culture with little or no syncytium formation.

In some embodiments, the recombinant VSV comprises a spike glycoprotein S of SARS-CoV-2 comprising an amino acid sequence set forth in SEQ ID NOs: 1, 3, 5, 7, 9, 11, or 13. In some embodiments, the recombinant VSV comprises a spike glycoprotein S of SARS-CoV-2 comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or 100%) identify with the amino acid sequence set forth in SEQ ID NOs: 1, 3, 5, 7, 9, 11, or 13.

In some embodiments, the recombinant VSV comprises a genome comprising a nucleic acid encoding a spike glycoprotein S of SARS-CoV-2 comprising an amino acid sequence set forth in SEQ ID NOs: 1, 3, 5, 7, 9, 11, or 13. In some embodiments, the recombinant VSV comprises a genome comprising a nucleic acid encoding a spike glycoprotein S of SARS-CoV-2 comprising an amino acid sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or 100%) identify with the amino acid sequence set forth in SEQ ID NOs: 1, 3, 5, 7, 9, 11, or 13.

In some embodiments, the recombinant VSV comprises a genome comprising a nucleic acid comprising a nucleotide sequence set forth in SEQ ID NOs: 2, 4, 6, 8, 10, 12, or 14. In some embodiments, the recombinant VSV comprises a genome comprising a nucleic acid comprising a nucleotide sequence having at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%, or 100%) identify with the nucleotide sequence set forth in SEQ ID NOs: 2, 4, 6, 8, 10, 12, or 14.

In some embodiments, the recombinant VSV lacks native VSV entry glycoprotein G. In some embodiments, the recombinant VSV comprises a genome that lacks native VSV entry glycoprotein gene, G. In certain embodiments, the native VSV entry glycoprotein gene, G, is replaced with the nucleic acid encoding spike glycoprotein S of SARS-CoV-2 in the geome of the recombinant VSV.

The recombinant VSV may further comprises a genome comprising a nucleic acid encoding a detectable label (e.g., a reporter). In some embodiments, the reporter is a fluorescent protein, including but are not limited to, for example, an enhanced green fluorescent protein (eGFP), GFP, a blue fluorescent protein (BFP), a yellow fluorescent protein (YFP), a red fluorescent protein (RFP), an orange fluorescent protein (OFP), etc.

In some embodiments, the recombinant VSV viral particle incorporates the spike glycoprotein S of SARS-CoV-2. In some embodiments, the recombinant VSV is replication-competent. In some embodiments, the recombinant VSV is capable of efficient spread in tissue culture with little or no syncytium formation.

In some embodiments, the recombinant VSV closely resembles SARS-CoV-2 in its entry-related properties. For example, in some embodiments, entry of the recombinant VSV in cells depends on level and/or activity of cysteine cathepsins, and/or endosomal acid pH of the cells. In some embodiments, entry of the recombinant VSV in cells depends on level and/or activity of TMPRSS2 of the cells. In some embodiments, entry of the recombinant VSV in cells depends on level and/or activity of ACE2 of the cells, and/or interaction between the spike glycoprotein S and the ACE2 receptor.

In some embodiments, the recombinant VSV is attenuated. In some embodiments, the recombinant VSV is capable of inducing immune responses (e.g., antibody production) to SARS-CoV-2 in a subject.

The present invention also provides methods for making a recombinant VSV vector disclosed herein comprising growing a cell comprising said VSV vector under conditions whereby VSV is produced; and optionally isolating said VSV. In some embodiments, the VSV vector is replication defective and the host cells comprising the VSV protein function essential for VSV replication such that said VSV vector is capable of replication in said host cell.

The present invention also provides host cells comprising (i.e., transformed, transfected or infected with) the VSV vectors or particles described herein. Both prokaryotic and eukaryotic host cells, including insect cells, can be used as long as sequences requisite for maintenance in that host, such as appropriate replication origin(s), are present. For convenience, selectable markers are also provided. Host systems are known in the art and need not be described in detail herein. Prokaryotic host cells include bacterial cells, for example, E. coli. B. subtilis, and mycobacteria. Among eukaryotic host cells are yeast, insect, avian, plant, C. elegans (or nematode) and mammalian host cells. Examples of fungi (including yeast) host cells are S. cerevisiae, Kluyveromyces Lactis (K. lactis), species of Candida including C. alhicans and C. glabrata, Aspergillus nidulans, Schizosaccharomyces pombe (S. pombe), Pichia pastoris, and Yarrowia lipolytica. Examples of mammalian cells are COS cells, mouse L cells, LNCaP cells, Chinese hamster ovary (CHO) cells, human embryonic kidney (HEK) cells, and African green monkey cells. Xenopus laevis oocytes, or other cells of amphibian origin, may also be used.

### III. Immunogenic Compositions

In some aspects, provided herein are pharmaceutical compositions (e.g., a vaccine composition) comprising one or more recombinant VSV vectors described herein (e.g., one or more recombinant VSV vectors comprising a nucleic acid encoding a spike glycoprotein S of SARS-CoV-2 described herein). In some aspects, provided herein are pharmaceutical compositions (e.g., a vaccine composition) comprising one or more recombinant VSV viruses described herein (e.g., one or more recombinant VSV viruses comprising a spike glycoprotein S of SARS-CoV-2 described herein). In some embodiments, the compositions described above further comprise an adjuvant. In some embodiments, the compositions described above further comprises a pharmaceutically acceptable carrier.

The pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association a recombinant VSV vector and/or a recombinant VSV virus described herein with the adjuvant, carrier and, optionally, one or more accessory ingredients. In general, the formulations are prepared by uniformly and intimately bringing into association a recombinant VSV vector and/or a recombinant VSV virus described herein with liquid carriers, or finely divided solid carriers, or both, and then, if necessary, shaping the product.

Pharmaceutical compositions suitable for parenteral administration comprise recombinant VSV vectors and/or recombinant VSV virus described herein in combination with an adjuvant, as well as one or more pharmaceutically-acceptable sterile isotonic aqueous or nonaqueous solutions, dispersions, suspensions or emulsions, or sterile powders which may be reconstituted into sterile injectable solutions or dispersions just prior to use. Such pharmaceutical compositions may contain sugars, alcohols, antioxidants, buffers, bacteriostats, solutes that render the formulation isotonic with the blood of the intended recipient, or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

In some embodiments, the pharmaceutical composition described, when administered to a subject, can elicit an immune response against SARS-CoV-2 infection. Such pharmaceutical compositions may be useful as vaccine compositions for prophylactic and/or therapeutic treatment of COVID-19.

In some embodiments, the pharmaceutical composition further comprises a physiologically acceptable adjuvant. In some embodiments, the adjuvant employed provides for increased immunogenicity of the pharmaceutical composition. Such a further immune response stimulating compound or adjuvant may be (i) admixed to the pharmaceutical composition according to the invention after reconstitution of the recombinant VSV vectors or viruses and optional emulsification with an oil-based adjuvant, (ii) may be part of the reconstitution composition of the invention defined above, (iii) may be physically linked to the recombinant VSV vector(s) or virus(es) to be reconstituted or (iv) may be administered separately to the subject, mammal or human, to be treated. The adjuvant may be one that provides for slow release of antigen (e.g., the adjuvant may be a liposome), or it may be an adjuvant that is immunogenic in its own right thereby functioning synergistically with antigens (i.e., antigens present in the SARS-CoV-2 virus). For example, the adjuvant may be a known adjuvant or other substance that promotes antigen uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, Bacto-Adjuvant, synthetic polymers such as poly amino acids and co-polymers of amino acids, saponin, paraffin oil, and muramyl dipeptide. In some embodiments, the adjuvant is Adjuvant 65, α-GalCer, aluminum phosphate, aluminum hydroxide, calcium phosphate, β-Glucan Peptide, CpG DNA, GM-CSF, GPI-0100, IFA, IFN-γ, IL-17, lipid A, lipopolysaccharide, Lipovant, Montanide, N-acetyl-muramyl-L-alanyl-D-isoglutamine, Pam3CSK4, quil A, trehalose dimycolate or zymosan.

In some embodiments, the adjuvant is an immunomodulatory molecule. For example, the immunomodulatory molecule may be a recombinant protein cytokine, chemokine, or immunostimulatory agent or nucleic acid encoding cytokines, chemokines, or immunostimulatory agents designed to enhance the immunologic response.

Examples of immunomodulatory cytokines include interferons (e.g., IFNα, IFNβ and IFNγ), interleukins (e.g., IL-1, IL-2, IL-3, IL-4, IL-5, IL-6, IL-7, IL-8, IL-9, IL-10, IL-12, IL-17 and IL-20), tumor necrosis factors (e.g., TNFα and TNFβ), erythropoietin (EPO), FLT-3 ligand, gIp10, TCA-3, MCP-1, MIF, MIP-1.alpha., MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In some embodiments, an immunomodulatory chemokine that binds to a chemokine receptor, i.e., a CXC, CC, C, or CX3C chemokine receptor, also may be included in the compositions provided here. Examples of chemokines include, but are not limited to, Mip1α, Mip-1β, Mip-3α (Larc), Mip-3β, Rantes, Hcc-1, Mpif-1, Mpif-2, Mcp-1, Mcp-2, Mcp-3, Mcp-4, Mcp-5, Eotaxin, Tarc, Elc, 1309, IL-8, Gcp-2 Gro-α, Gro-β, Gro-γ, Nap-2, Ena-78, Gcp-2, Ip-10, Mig, I-Tac, Sdf-1, and Bca-1 (B1c), as well as functional fragments of any of the foregoing.

When taken up by a cell (e.g., cells of human airway origin), a recombinant VSV vector may be present in the cell as an extrachromosomal molecule and/or may integrate into the chromosome. The recombinant VSV vector may be introduced into cells in the form of a plasmid which may remain as separate genetic material. Alternatively, linear recombinant VSV vectors that may integrate into the chromosome may be introduced into the cell. Optionally, when introducing a recombinant VSV vector into a cell, reagents which promote the recombinant VSV vector integration into chromosomes may be added.

### IV. Uses and Methods

a. Screening Methods

A recombinant VSV vector or a recombinant VSV virus described herein can be used to screen for agents that reduces, inhibits, prevents and/or treats SARS-CoV-2 infection.

Accordingly, in some aspects, provide herein are methods for identifying an agent that prevents and/or treats SARS-CoV-2 infection, comprising: (a) contacting cells with a recombinant VSV vector or a recombinant VSV virus described herein, and a test agent; and (b) identifying the test agent that reduces the recombinant VSV infection of the cells, thereby identifying an agent that prevents and/or treats SARS-CoV-2 infection.

The test agent may be added before, concurrently, or after contacting the cells with the recombinant VSV vector or the recombinant VSV virus. In one embodiment, the test agent reduces the recombinant VSV infection of the cells compared with that of the cells treated with a control agent (e.g., an agent that is known to have no effects on the recombinant VSV infection). In another embodiment, the test agent reduces the recombinant VSV infection of the cells compared with that of the untreated cells. In still another embodiment, the test agent reduces the recombinant VSV infection of the cells compared with a reference number. In certain embodiments, the test agent reduces spike glycoprotein-dependent entry of the recombinant VSV. In certain embodiments, the test agent reduces interaction between the spike glycoprotein S and the ACE2 receptor.

Agents to be tested for their ability to reduce recombinant VSV infection of cells, can be produced, for example, by bacteria, yeast or other organisms (e.g., natural products), produced chemically (e.g., small molecules, including peptidomimetics), or produced recombinantly. In some embodiments, the test agent may be derived from natural sources, or it may be partly or wholly synthetically or recombinantly produced. Agents for use with the above-described methods may be selected from the group of compounds consisting of lipids, carbohydrates, polypeptides, peptidomimetics, peptide-nucleic acids (PNAs), small molecules, natural products, antibodies or antigen-binding fragments thereof, aptamers and polynucleotides. In certain embodiments, the test agent is a small molecule. In certain embodiments, the test agent is a small-molecule inhibitor or an antibody targeting the spike glycoprotein S. In a specific embodiment, the test agent comprises an antibody and/or intrabody, or an antigen binding fragment thereof, which specifically binds to the spike glycoprotein S of SARS-CoV-2. In certain embodiments, the test agent is a blood, serum or plasma sample from a COVID-19 convalescent.

In some embodiments, a test agent that prevents and/or treats SARS-CoV-2 infection may be identified by contacting one or more candidate test agents, such as one or more candidate small molecules, antibodies or antigen-binding fragments thereof, with cells and a recombinant VSV vector or a recombinant VSV virus described herein, and assessing whether each of the one or more candidate test agents reduces, such as inhibits, the recombinant VSV infection of the cells. The methods may be performed in vitro, ex vivo or in vivo.

In some embodiments, the methods include contacting a plurality or library of test agents, such as a plurality or library of small molecule compounds or antibodies, with cells and a recombinant VSV vector or a recombinant VSV virus described herein and identifying or selecting test agents that specifically reduces, such as inhibits, the recombinant VSV infection of the cells. In some embodiments, a library or collection containing a plurality of different test agents, such as a plurality of different small molecule compounds or a plurality of different antibodies, may be screened or assessed for effects on the recombinant VSV infection.

In some embodiments, screening methods may be employed in which a plurality of candidate test agents, such as a library or collection of candidate test agents, are individually contacted with the cells, either simultaneously or sequentially. Library members that specifically reduces the recombinant VSV infection may be identified or selected. In some embodiments, the library or collection of candidate test agents may contain at least 2, 5, 10, 100, 10³, 10⁴, 10⁵, 10⁶, 10⁷, 10⁸, 10⁹, or more different test agents.

Agents/molecules (candidate molecules) to be screened can be provided as mixtures of a limited number of specified compounds, or as compound libraries, peptide libraries and the like. Agents/molecules to be screened may also include all forms of antisera, antisense nucleic acids, etc., that can modulate complex activity or formation. Exemplary candidate molecules and libraries for screening are set forth below.

In certain embodiments, the compounds are screened in pools. Once a positive pool has been identified, the individual molecules of that pool are tested separately. In certain embodiments, the pool size is at least 2, at least 5, at least 10, at least 25, at least 50, at least 75, at least 100, at least 150, at least 200, at least 250, or at least 500 compounds.

In certain embodiments, the invention provides screening assays using chemical libraries for molecules which modulate, e.g., inhibit, the recombinant VSV infection of cells. The chemical libraries can be peptide libraries, peptidomimetic libraries, chemically synthesized libraries, recombinant, e.g., phage display libraries, and in vitro translation-based libraries, other non-peptide synthetic organic libraries, etc.

Exemplary libraries are commercially available from several sources (ArOule, Tripos/PanLabs, ChemDesign, and Pharmacopoeia). In some cases, these chemical libraries are generated using combinatorial strategies that encode the identity of each member of the library on a substrate to which the member compound is attached, thus allowing direct and immediate identification of a molecule that is an effective modulator. Thus, in many combinatorial approaches, the position on a plate of a compound specifies that compound's composition. Also, in one example, a single plate position may have from 1-20 chemicals that can be screened by administration to a well containing the interactions of interest. Thus, if modulation is detected, Smaller and Smaller pools of interacting pairs can be assayed for the modulation activity. By Such methods, many candidate molecules can be screened.

Many diversity libraries suitable for use are known in the art and can be used to provide compounds to be tested according to the present invention. Alternatively, libraries can be constructed using standard methods. Chemical (synthetic) libraries, recombinant expression libraries, or polysome based libraries are exemplary types of libraries that can be used.

The libraries can be constrained or semirigid (having some degree of structural rigidity), or linear or non-constrained. The library can be a cDNA or genomic expression library, random peptide expression library or a chemically synthesized random peptide library, or non-peptide library. Expression libraries are introduced into the cells in which the assay occurs, where the nucleic acids of the library are expressed to produce their encoded proteins.

In one embodiment, peptide libraries that can be used in the present invention may be libraries that are chemically synthesized in vitro. Examples of such libraries are given in Houghten et al. (1991) Nature 354:84-86, which describes mixtures of free hexapeptides in which the first and second residues in each peptide were individually and specifically defined; Lam et al. (1991) Nature 354:82-84, which describes a “one bead, one peptide’ approach in which a solid phase split synthesis scheme produced a library of peptides in which each bead in the collection had immobilized thereon a single, random sequence of amino acid residues; Medynski, 1994, Bio/Technology 12:709-710, which describes split synthesis and T-bag synthesis methods; and Gallop et al. (1994) J. Medicinal Chemistry 37(9): 1233-1251. Simply by way of other examples, a combinatorial library may be prepared for use, according to the methods of Ohlmeyer et al. (1993) Proc. Natl. Acad. Sci. USA 90:10922-10926; Erb et al. (1994) Proc. Natl. Acad. Sci. USA 91:11422-11426; Houghten et al. (1992) Biotechniques 13:412; Jayawickreme et al. (1994) Proc. Natl. Acad. Sci. USA 91:1614-1618; or Salmon et al. (1993) Proc. Natl. Acad. Sci. USA 90:11708-11712. PCT Publication No. WO 93/20242 and Brenner and Lerner. (1992) Proc. Natl. Acad. Sci. USA 89:5381-5383 describe “encoded combinatorial chemical libraries,” that contain oligonucleotide identifiers for each chemical polymer library member.

Antibody libraries can be constructed by a number of processes (see, e.g., WO 00/70023). Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another embodiment, the variation is introduced into CDR1 and CDR2, e.g., of a heavy chain variable domain. Any combination is feasible. In one process, antibody libraries are constructed by inserting diverse oligonucleotides that encode CDRs into the corresponding regions of the nucleic acid. The oligonucleotides can be synthesized using monomeric nucleotides or trinucleotides. For example, Knappik et al. (2000) J. Mol. Biol. 296:57-86 describes a method for constructing CDR encoding oligonucleotides using trinucleotide synthesis and a template with engineered restriction sites for accepting the oligonucleotides.

In some cases, the antibodies or antigen-binding fragments thereof are IgM-derived antibodies or antigen-binding fragments. In some aspects, the test agents are present in a native antibody library. In some embodiments, the test agent is a single chain variable fragment (scFv). In some aspects, the test agent contain one or more amino acid mutations compared to a parent antibody or antigen-binding fragment thereof. In some embodiments, the one or more amino acid mutations include a mutation or mutations in a complementarity determining region (CDR) or CDRs of the antibody or antigen-binding fragment thereof.

In some instances, the test agents are present in a display library. In some examples, the display library is a cell surface display library, a phage display library, ribosome display library, mRNA display library, or a dsDNA display library.

In many drug screening programs which test libraries of compounds and natural extracts, high throughput assays are desirable in order to maximize the number of compounds surveyed in a given period of time.

A variety of assay formats will suffice and, in light encompassed by the present disclosure, those not expressly described herein may nevertheless be comprehended by one of ordinary skill in the art based on the teachings herein. Assay formats for analyzing activity of a test agent on the recombinant VSV infection, may be generated in many different forms, and include, e.g., cell-based assays which utilize intact cells (e.g., in a prokaryotic or eukaryotic cell culture system). Advantages to conduct the screening methods described herein in an intact cell includes the ability to screen for modulators of SARS-CoV-2 infection, which are functional in an environment more closely approximating that which therapeutic use of the modulator would require, including the ability of the agent to gain entry into the cell. Furthermore, certain of the in vivo embodiments of the assay are amenable to high through-put analysis of candidate agents.

In some embodiments, in the whole cell embodiments of the subject assay, the recombinant VSV vector or virus described herein comprise a reporter gene which can provide, upon expression, a selectable marker. Such embodiments of the subject assay are particularly amenable to high through-put analysis in that the number of or percentage of the cells that express the selectable marker can provide a simple measure of the recombinant VSV viral infection.

A reporter gene includes any gene that expresses a detectable gene product, which may be RNA or protein. Preferred reporter genes are those that are readily detectable. For example, the reporter gene is a fluorescent protein, including, but are not limited to, eGFP, GFP, BFP, YFP, RFP, or OFP. The reporter gene may also be included in the construct in the form of a fusion gene with a gene that includes desired transcriptional regulatory sequences or exhibits other desirable properties. For instance, the product of the reporter gene can be an enzyme which confers resistance to an antibiotic or other drug, or an enzyme which complements a deficiency in the host cell.

The amount of transcription from the reporter gene may be measured using any method known to those of skill in the art to be suitable. For example, specific mRNA expression may be detected using Northern blots or specific protein product may be identified by a characteristic stain, western blots or an intrinsic activity. In certain embodiments, the product of the reporter gene is detected by an intrinsic activity associated with that product. For instance, the reporter gene may encode a gene product that, by enzymatic activity, gives rise to a detection signal based on color, fluorescence, or luminescence. Therefore, modulation of the recombinant VSV infection may be quantified using, for example, detectably labeled proteins such as radiolabeled, fluorescently labeled, or enzymatically labeled proteins, by immunoassay, or by chromatographic detection.

In some embodiments, other methods of detecting the recombinant VSV infection of cells may be used. For example, the recombinant VSV infection may be determined by quantifying cells that contain nucleic acid or protein of the spike glycoprotein S, or other recombinant VSV virus specific nucleic acids or proteins, or fragments thereof. In some embodiments, the recombinant VSV infection may be determined by detecting specific cell phenotypes.

In some embodiments, the screening methods described herein are used in combination with one or more screening assays based on authentic SARS-CoV-2 virus.

In certain embodiments of the invention, the screening method further comprises determining the structure of the candidate molecule. The structure of a candidate molecule can be determined by any technique known to the skilled artisan.

In some embodiments, the test agent is used to prevent and/or treat SARS-CoV-2 infection.

b. Diagnostic Methods

In some aspects, provided herein are diagnostic methods for determining whether a subject has exposure to and/or protection from SARS-CoV-2 comprising: (a) contacting cells with a recombinant VSV vector described herein (e.g., a recombinant VSV vector comprising a nucleic acid encoding a spike glycoprotein S of SARS-CoV-2) or a recombinant VSV virus described herein (e.g., a recombinant VSV virus comprising a spike glycoprotein S of SARS-CoV-2), and a sample obtained from the subject; and (b) detecting the determining the recombinant VSV infection of the cells; wherein the absence of or a lower level of the recombinant VSV infection of the cells compared to a control level indicates that the subject has exposure to and/or protection from SARS-CoV-2.

In some aspects, provided herein are diagnostic methods for determining whether a subject has neutralizing antibodies to the spike glycoprotein S of SARS-CoV-2, comprising: (a) contacting cells with a recombinant VSV vector described herein (e.g., a recombinant VSV vector comprising a nucleic acid encoding a spike glycoprotein S of SARS-CoV-2) or a recombinant VSV virus described herein (e.g., a recombinant VSV virus comprising a spike glycoprotein S of SARS-CoV-2), and a sample obtained from the subject; and (b) detecting the determining the recombinant VSV infection of the cells; wherein the absence of or a lower level of the recombinant VSV infection of the cells compared to a control level indicates that the subject has neutralizing antibodies to the spike glycoprotein S of SARS-CoV-2.

In some embodiments, the recombinant VSV infection is assessed by determining the number or percentage of cells that express the reporter of the recombinant VSV vector or the recombinant VSV virus (e.g., by flow cytometry or microscopy). In some embodiments, the sample is added before, concurrently, or after contacting the cells with the recombinant VSV vector or virus. The control level may be a reference number, a level of the recombinant VSV infection of cells treated with a sample obtained from a subject without exposure to SARS-CoV-2, or a level of the recombinant VSV infection of untreated cells. In some embodiments, the sample is a blood, serum, or plasma sample.

c. Prophylactic Methods

In one aspect, the present invention provides a method for preventing COVID-19 (i.e., a SARS-CoV-2 infection) in a subject. Subjects at risk for COVID-19 that would benefit from treatment with the claimed agents or methods can be identified, for example, by any or a combination of diagnostic or prognostic assays known in the art. Administration of a prophylactic agent can occur prior to the manifestation of symptoms associated with COVID-19. The appropriate agent used for treatment (e.g. antibodies, peptides, fusion proteins or small molecules) can be determined based on clinical indications and can be identified, e.g., using screening assays described herein.

d. Therapeutic Methods

In some aspects, provided herein are methods for treating COVID-19 (i.e., a SARS-CoV-2 infection), and/or for inducing an immune response against SARS-CoV-2 virus. In certain embodiments, the method comprises administering to a subject an immunogenic composition described herein.

The prophylactic and/or therapeutic methods described herein may be used to treat any subject in need thereof, including any subject who has COVID-19, who has had COVID-19 and/or who is predisposed to COVID-19. For example, in some embodiments, the subject has a COVID-19. In some embodiments, the subject has undergone treatments for COVID-19. In some embodiments, the subject is predisposed to COVID-19 due to age, or having a compromised immune system or other serious underlying medical conditions that predisposes the subject to COVID-19.

The pharmaceutical compositions disclosed herein may be delivered by any suitable route of administration, including orally and parenterally. In certain embodiments the pharmaceutical compositions are delivered generally (e.g., via oral or parenteral administration). In specific embodiments, the pharmaceutical compositions is administered by subcutaneous injection.

The dosage of the subject agent may be determined by reference to the plasma concentrations of the agent. For example, the maximum plasma concentration (Cmax) and the area under the plasma concentration-time curve from time 0 to infinity (AUC (0-4)) may be used. Dosages include those that produce the above values for Cmax and AUC (0-4) and other dosages resulting in larger or smaller values for those parameters.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the agents employed in the pharmaceutical composition at levels lower than that required in order to achieve the desired therapeutic effect and gradually increase the dosage until the desired effect is achieved.

In general, a suitable daily dose of an agent described herein will be that amount of the agent which is the lowest dose effective to produce a therapeutic effect. Such an effective dose will generally depend upon the factors described above.

In some embodiments, the immunogenic composition comprises an amount of a recombinant VSV vector descried herein or a recombinant VSV virus described herein in combination with an adjuvant that constitutes a pharmaceutical dosage unit. A pharmaceutical dosage unit is defined herein as the amount of active ingredients (e.g., a recombinant VSV vector descried herein or a recombinant VSV virus described herein and/or adjuvant) that is applied to a subject at a given time point. A pharmaceutical dosage unit may be applied to a subject in a single volume, e.g., a single shot, or may be applied in 2, 3, 4, 5 or more separate volumes or shots that are applied at different locations of the body, for instance in the right and the left limb. Reasons for applying a single pharmaceutical dosage unit in separate volumes may be multiples, such as avoid negative side effects, avoiding antigenic competition and/or composition analytics considerations. It is to be understood herein that the separate volumes of a pharmaceutical dosage may differ in composition, i.e., may comprise different kinds or composition of active ingredients and/or adjuvants.

A pharmaceutical dosage unit may be an effective amount or part of an effective amount. An “effective amount” is to be understood herein as an amount or dose of active ingredients required to prevent and/or reduce the symptoms of a disease (e.g., COVID-19) relative to an untreated patient. The effective amount of active compound(s) used to practice the present invention for preventive and/or therapeutic treatment of COVID-19 varies depending upon the manner of administration, the age, body weight, and general health of the subject. Ultimately, the attending physician or veterinarian will decide the appropriate amount and dosage regimen. Such amount is referred to as an “effective” amount. This effective amount may also be the amount that is able to induce an effective immune response in the subject to be treated, for example, the amount that is able to induce production of an effective amount of neutralizing antibodies to the spike glycoprotein S of SARS-CoV-2.

In one aspect, provided herein is a method of eliciting in a subject an immune response to a cell that is infected with SARS-CoV-2 virus. The method comprises administering to the subject a pharmaceutical composition described herein, wherein the pharmaceutical composition, when administered to the subject, elicits an immune response to the cell that is infected with SARS-CoV-2 virus.

Generally, the immune response may include a humoral immune response, a cell-mediated immune response, or both.

A humoral response may be determined by a standard immunoassay for antibody levels in a serum sample from the subject receiving the pharmaceutical composition. A cellular immune response is a response that involves T cells and may be determined in vitro or in vivo. For example, a general cellular immune response may be determined as the T cell proliferative activity in cells (e.g., peripheral blood leukocytes (PBLs)) sampled from the subject at a suitable time following the administering of a pharmaceutical composition. Following incubation of e.g., PBMCs with a stimulator for an appropriate period, [³H]thymidine incorporation may be determined. The subset of T cells that is proliferating may be determined using flow cytometry.

In certain aspects, the methods provided herein include administering to both human and non-human mammals. Veterinary applications also are contemplated. In some embodiments, the subject may be any living organism in which an immune response may be elicited. Examples of subjects include, without limitation, humans, livestock, dogs, cats, mice, rats, and transgenic species thereof.

In some embodiments, the pharmaceutical composition may be administered at any time that is appropriate. For example, the administering may be conducted before or during treatment of a subject having a COVID-19, and continued after the SARS-CoV-2 infection becomes clinically undetectable. The administering also may be continued in a subject showing signs of recurrence.

In some embodiments, the pharmaceutical composition may be administered in a therapeutically or a prophylactically effective amount. Administering the pharmaceutical composition to the subject may be carried out using known procedures, and at dosages and for periods of time sufficient to achieve a desired effect.

In some embodiments, the pharmaceutical composition may be administered to the subject at any suitable site. The route of administering may be parenteral, intramuscular, subcutaneous, intradermal, intraperitoneal, intranasal, intravenous (including via an indwelling catheter), via an afferent lymph vessel, or by any other route suitable in view of the subject's condition. Preferably, the dose will be administered in an amount and for a period of time effective in bringing about a desired response, be it eliciting the immune response or the prophylactic or therapeutic treatment of the SARS-CoV-2 infection and/or symptoms associated therewith.

The pharmaceutical composition may be given subsequent to, preceding, or contemporaneously with other therapies including therapies that also elicit an immune response in the subject. For example, the subject may previously or concurrently be treated by other forms of immunomodulatory agents, such other therapies preferably provided in such a way so as not to interfere with the immunogenicity of the compositions described herein.

Administering may be properly timed by the care giver (e.g., physician, veterinarian), and may depend on the clinical condition of the subject, the objectives of administering, and/or other therapies also being contemplated or administered. In some embodiments, an initial dose may be administered, and the subject monitored for an immunological and/or clinical response. Suitable means of immunological monitoring include using patient's peripheral blood lymphocyte (PBL) as responders and immunogenic compositions herein as stimulators. An immunological reaction also may be determined by a delayed inflammatory response at the site of administering. One or more doses subsequent to the initial dose may be given as appropriate, typically on a monthly, semimonthly, or a weekly basis, until the desired effect is achieved. Thereafter, additional booster or maintenance doses may be given as required, particularly when the immunological or clinical benefit appears to subside.

### V. Kits

The present invention also encompasses kits. For example, the kit may comprise recombinant VSV vectors comprising a nucleic acid sequences encoding a spike glycoprotein S of SARS-CoV-2, recombinant VSV viruses comprising a genome comprising a nucleic acid sequences encoding a spike glycoprotein S of SARS-CoV-2, recombinant VSV viruses comprising a spike glycoprotein S of SARS-CoV-2, immunogenic composition comprising recombinant VSV vectors or viruses described herein, adjuvants, and combinations thereof, packaged in a suitable container and may further comprise instructions for using such reagents. The kit may also contain other components, such as administration tools packaged in a separate container.

The disclosure is further illustrated by the following examples which should not be construed as limiting. The contents of all references, patents and published patent applications cited throughout this application, as well as the Figures, are incorporated herein by reference.

### EXAMPLES

### Example 1: Materials and Methods for Example 2

### A. Experimental Model and Subject Details

Cells. Human hepatoma Huh7.5.1 (received from Dr. Jan Carette; originally from Dr. Frank Chisari) and 293FT (ThermoFisher) cells were cultured in Dulbecco's Modified Eagle Medium (DMEM high glucose, Gibco) supplemented with 10% heat-inactivated fetal bovine serum (FBS, Atlanta Biologicals), 1% Penicillin/Streptomycin (P/S, Gibco) and 1% Gluta-MAX (Gibco). The African vervet monkey kidney Vero cells and baby hamster kidney BHK21 cells were maintained in DMEM (high glucose) supplemented with 2% heat-inactivated FBS, 1% P/S and 1% Gluta-MAX. Vero-E6 cells were grown in Minimum Essential Medium (MEM) supplemented in 10% FBS and Gentamicin (all from Sigma). A549 cells were maintained in DMEM (high glucose) supplemented with 10% heat-inactivated FBS, 1% P/S and 1% Gluta-MAX. These cell lines were passaged every 2-3 days using 0.05% Trypsin/EDTA solution (Gibco). Calu3 cells were cultured in Eagle's Minimum Essential Medium (EMEM, ATCC) supplemented with 10% heat-inactivated FBS, 1% P/S, and 1% Gluta-MAX and passaged weekly using 0.05% Trypsin/EDTA solution.

Generation of cells overexpressing hACE2 or TMPRSS2. Human ACE2 and TMPRSS2 coding sequences were PCR-amplified from the hACE2 plasmid Addgene #1786 (a generous gift from Hyeryun Choe) and TMPRSS2 plasmid Addgene #53887 (a generous gift from Roger Reeves), respectively and cloned into a retroviral pBabe-puro vector. Retroviruses were produced by transfecting 293FT cells with the hACE2 and TMPRSS2 expressing pBabe-puro plasmids along with those expressing the Moloney murine leukemia virus (MMLV) gag-pol and VSV G proteins. Retroviral supernatants passed through a 0.45-μm filter were used to transduce BHK21, Vero cells or A549 cells. Transfected cells were selected with puromycin (2 μg/ml).

Convalescent serum samples. Serum samples were collected from healthy adult volunteers residing in Westchester County, NY who had recovered from COVID-19 in April 2020. Patients had reported a positive nasopharyngeal swab by PCR for SARS-CoV-2 during illness and had been asymptomatic for at least 14 days prior to sample collection. After obtaining informed consent, serum was obtained by venipuncture (BD Vacutainer, serum), centrifuged, aliquoted and stored at −80° C. prior to use. The sera were heat-inactivated at 56° C. for 30 minutes and stored at 4° C. prior to analysis. Protocol approval was obtained by the Institutional Review Board (IRB) of the Albert Einstein College of Medicine.

Generation of rVSV-SARS-CoV-2. A plasmid encoding the VSV antigenome was modified to replace its native glycoprotein, G, with the full-length wild-type S glycoprotein gene of the Wuhan-Hu-1 isolate of SARS-CoV-2 (GenBank MN908947.3). The VSV antigenome also encodes for an eGFP reporter gene as a separate transcriptional unit. Plasmid-based rescue of the rVSV was carried out as described previously (Kapadia et al. (2005) Virology 340:174-182; Whelan et al. (1995) Proc Natl Acad Sci USA 92:8388-8392; Wong et al. (2010) J. Virol. 84:163-175). Briefly, 293FT cells were transfected with the VSV antigenome plasmid along with plasmids expressing T7 polymerase and VSV N, P, M, G and L proteins by using polyethylenimine. Supernatants from the transfected cells were transferred to Huh7.5.1 cells every day (day 2-7 post-transfection) till the appearance of eGFP-positive cells. The poorly spreading virus was initially propagated by cell subculture. RNA was isolated from viral supernatants at different passages and Sanger sequencing was used to verify S gene sequences. A passage #9 viral stock was plaque-purified on Vero cells. Supernatants were aliquoted and stored at −80° C. The generation of rVSV-SARS-CoV-2 S and its use in tissue culture at biosafety level 2 was approved by the Environmental Health and Safety Department and the Institutional Biosafety Committee at Albert Einstein College of Medicine.

SARS-CoV-2 stock preparation. All work with authentic SARS-CoV-2 was completed in BSL-3 laboratories at USAMRIID in accordance with federal and institutional biosafety standards and regulations. Vero-76 cells were inoculated with SARS-CoV-2 (GenBank MT020880.1) at a MOI=0.01 and incubated at 37° C. with 5% CO2 and 80% humidity. At 50 hours post-infection, cells were frozen at −80° C. for 1 hour, allowed to thaw at room temperature, and supernatants were collected and clarified by centrifugation at −2,500×g for 10 min. Clarified supernatant was aliquoted and stored at −80° C. Sequencing data from this virus stock indicated a single mutation in the spike glycoprotein (H655Y) relative to Washington state isolate MT020880.1.

SARS-CoV2 spike glycoprotein RBD expression and purification. The pCAGGS SARS-CoV2 RBD plasmid (a generous gift from Florian Krammer) was used for the expression of recombinant RBD as previously described (Amanat et al. (2020) Nat. Med. doi.org/10.1038/s41591-020-0913-5; Stadlbauer et al. (2020) Curr. Protoc. Microbiol. 57:e100). FreeStyle 293F cells (ThermoFisher Scientific) were transfected with the plasmid DNA diluted in PBS (0.67 μg total plasmid DNA per ml of culture) using polyethylenimine (Polysciences, Inc.) at a DNA-to-PET ratio of 1:3. At 6 days post-transfection, cultures were harvested by centrifugation at 4,000×g for 20 min, and supernatant was incubated with Ni-NTA resin (GoldBio) for 2 h at 4° C. Resin was collected in columns by gravity flow, washed with a wash buffer (50 mM Tris HCl pH 8.0, 250 mM NaCl, 20 mM Imidazole) and eluted with an elution buffer (50 mM Tris HCl pH 8.0, 250 mM NaCl, 250 mM Imidazole). Eluant was concentrated in Amicon centrifugal units (EMD Millipore) and buffer was exchanged into the storage buffer (50 mM Tris HCl pH 8.0, 250 mM NaCl). Protein was analyzed by SDS-PAGE, aliquoted, and stored at −80° C.

SARS-CoV2 spike glycoprotein expression and purification. The pCAGGS SARS-CoV2 plasmid encoding stabilized S glycoprotein gene (a generous gift from Jason McLellan) was used for the expression of recombinant S protein as described previously (Wrapp et al. (2020) Science 367:1260-1263) with several modifications. ExpiCHO-S cells (ThermoFisher) were transiently transfected with plasmid DNA diluted in OptiPRO Serum-Free Medium (0.8 μg total DNA per ml of culture) using ExpiFectamine (ThermoFisher) at a DNA-to-ExpiFectamine ratio of 1:4. At 8 days post-transfection, cultures were harvested by centrifugation at 4,000×g for 20 min. Clarified supernatant was dialyzed in 50 mM Tris HCl pH 8.0, 250 mM NaCl at a clarified supernatant to dialysis buffer ratio of 1:25 prior affinity chromatography. Dialyzed supernatant was incubated with Ni-NTA resin (GoldBio) for 2 h at 4° C. Resin was collected in columns by gravity flow, washed with wash buffer (50 mM Tris HCl pH 8.0, 250 mM NaCl, 20 mM Imidazole) and eluted with elution buffer (50 mM Tris HCl pH 8.0, 250 mM NaCl, 250 mM imidazole). Eluate was concentrated in Amicon centrifugal units (EMD Millipore) and exchanged into a storage buffer (50 mM Tris HCl pH 8.0, 250 mM NaCl). Protein was analyzed by SDS-PAGE, aliquoted, and stored at −80° C.

### B. Method Details

Detection of S protein in rVSV-SARS-CoV-2. High-protein binding 96-well ELISA plates (Corning) were coated with 25 μl of concentrated rVSV-SARS-CoV-2 S or rVSV-EBOV (2.73 μg/ml) overnight at 4° C., and blocked with 3% nonfat dry milk in PBS (PBS-milk) for 1 h at 25° C. Plates were extensively washed and incubated with serum 18, serum 39 or negative serum diluted to 1:100 first then with serial 2-fold dilutions in PBS milk 1%-Tween 0.1% for 1 h at 25° C. Plates were washed three times and incubated with Goat anti-human IgG-HRP (#31410 Invitrogen) diluted 1:3000 (PBS milk 1%-Tween 0.1%) for 1 h at 25° C. and detected using 1-Step™ Ultra TMB-ELISA Substrate Solution (Thermo Fisher Scientific, Waltham, MA). Plates were read using a Cytation 5 imager (BioTek) at 450 nm.

NH₄Cl inhibition experiments. Huh7.5.1 cell monolayers were incubated for 1 hour with 20-100 mM NH₄Cl in DMEM. Next, pre-titrated rVSVs expressing EBOV GP or SARS-CoV-2 S were used to infect cells. Infection was scored 16-18 h later as described above.

Cathepsin Inhibitor experiments. Monolayers of Vero cells pre-treated for 1.5 hours at 37° C. with E-64 (37.5 or 75 μM), Z-Phe-tyr-dmk (FYdmk, 18.75 or 37.5 μM), or 1.5% DMSO (vehicle control) were infected with pre-titrated amounts of rVSVs carrying SARS-CoV 2 S, EBOV GP or VSV G. At 1 h post-infection, 20 mM NH₄Cl was added. Infected cells were fixed 16-18 h later and scored for infection as described above.

TMPRSS2 inhibitor experiments. Monolayers of Vero or Vero-TMPRSS2 cells pre-treated for 2 h at 37° C. with camostat mesylate (Tocris) or 1% DMSO (vehicle control) were infected with pre-titrated amounts of rVSV-SARS-CoV 2 S. At 1 h post-infection, 20 mM NH₄Cl was added to terminate viral entry. Infected cells were fixed 7 h later and scored for infection as described above.

Detection of hACE2 in BHK21 and A549 transfected cells. To stain for surface-expressed hACE2, BHK21, A549, BHK21-hACE2 and A549-hACE2 cells or the control cells were seeded onto fibronectin-coated glass coverslips were incubated with 0.4 μg/mL of hACE2-specific goat antibody (#AF933, R&D systems) at 4° C. in media containing 25 mM HEPES. Next, cells were washed with cold PBS, fixed with 4% paraformaldehyde, and blocked with buffer (2% (w/v) bovine serum albumin, 5% (v/v) glycerol, 0.2% (v/v) Tween20 in Ca²⁺/Mg²⁺-free PBS). Secondary donkey AlexaFluor 594-conjugated anti-goat IgG (#A32758 Invitrogen) was used to detect the hACE2 signal. Coverslips were mounted in Prolong with DAPI (Invitrogen) and imaged on an Axio Observer inverted microscope (Zeiss).

rVSV-SARS-CoV-2 S microneutralization assay. Serum samples were serially diluted and incubated with virus for 1 h at room temperature. Serum-virus mixtures were then added in duplicate to 384-well plates (Corning) containing Huh7.5.1 cells or 96-well plates (Corning) containing Vero cells. Plates were incubated for 16-18 h at 37° C. and 5% CO₂. Cells were fixed with 4% paraformaldehyde (Sigma), washed with PBS, and stored in PBS containing Hoechst-33342 (Invitrogen) at a dilution of 1:2,000 in. Viral infectivity was measured by automated enumeration of GFP-positive cells from captured images using a Cytation5 automated fluorescence microscope (BioTek) and analyzed using the Gen5 data analysis software (BioTek). The half-maximal inhibitory concentration (IC₅₀) of the mAbs was calculated using a nonlinear regression analysis with GraphPad Prism software.

Anti-hACE2 antibody blocking assay. Huh7.5.1 cells were seeded into a 384-well plate and Calu3 cells in a 96-well plate pre-coated with 1% gelatin/PBS, respectively. Next 15 day, goat anti-human ACE2 antibody (#AF933, R&D Systems) was serially diluted and applied to the cells. After 1 h incubation at 37° C. and 5% CO2, cells were infected with rVSV-SARS-CoV-2 S. At 16-18 h (Huh-7.5.1) or 8 h (Calu3) post-infection, cells were fixed and scored for infection as described above. Human gamma globulin (009-000-002) purchased from Jackson ImmunoResearch was used as negative control.

RBD competition assay. Monolayers of Huh7.5.1 cells in a 384-well plate were incubated with serial dilutions of recombinant RBD domain for 1 hour at 37° C. and 5% CO2. Cells were then infected with pre-titrated amounts of rVSV-SARS-CoV-2 or rVSV-EBOV GP and scored for infection 16-18 h later.

S-mediated antibody depletion assay. High-protein binding 96-well ELISA plates (Corning) coated with PBS alone or with 2 μg/ml of SARS-CoV-2 S protein in PBS overnight at 4° C. were blocked for 1 hour with 3% nonfat dry milk (Biorad) in PBS. Serum samples diluted in DMEM (1:50 dilution) were serially incubated 4 times for 1 h each at 37° C. on S protein-coated or control wells. The depleted sera were tested for their neutralization capacity as described above.

SARS-CoV-2 neutralization assay. Serially diluted serum samples were mixed with pre-diluted SARS-CoV-2 in infection media (EMEM/2% FBS/Gentamicin) and incubated for 1 hour at 37° C./5% CO₂/80% humidity. Virus/serum inoculum was added to Vero-E6 cells, seeded in 96 well plates, at a MOI of 0.4 and incubated for 1 hour at 37° C./5% CO₂/80% humidity. Virus/serum inoculum was removed and cells were washed with PBS prior to addition of culture media (MEM/10% FBS/Gentamicin). Following 24 h incubation at 37° C./5% CO₂/80% humidity, media was removed and cells were washed with PBS. PBS was removed and cells were submerged in 10% formalin for 24 h. Formalin was removed and cells were washed with PBS prior to permeabilization with 0.2% Triton-X for 10 minutes at room temperature. Cells were blocked for 2 h, then immunostained with SARS-1 nucleocapsid protein-specific antibody (Sino Biologic; 40143-V08B) and AlexaFluor 488 labeled secondary antibody. Cells were imaged using an Operetta (Perkin Elmer) high content imaging instrument and infected cells were determined using Harmony Software (Perkin Elmer).

Syncytia inhibition assay. Vero cells were infected with pre-titrated amounts of rVSV-SARS-CoV-2 S for 2 h at 37° C. and 5% C02. Following the removal of virus inocula, cells were washed with PBS to remove any residual virus and indicated dilutions of convalescent sera were applied to the infected cells. Cells were fixed, their nuclei were counterstained, and syncytia formation was imaged by eGFP expression at 16 h post-infection.

### C. Quantification and Statistical Analysis

The n number associated with each dataset in the figures indicates the number of biologically independent samples. The number of independent experiments and the measures of central tendency and dispersion used in each case are indicated in the figure legends. Dose-response neutralization curves were fit to a logistic equation by nonlinear regression analysis. Unless otherwise indicated in the figure legends, statistical comparisons were carried out by two-way ANOVA with a post hoc correction for family-wise error rate (Dunnett test for comparison of an untreated sample mean to treated sample means, Tukey test for all possible comparisons of sample means). Testing level (alpha) was 0.05 for all statistical tests. All analyses were carried out in GraphPad Prism 8.

### Example 2: Identification of S Gene Mutations that Facilitate Robust rVSV-SARS-CoV-2 S Replication

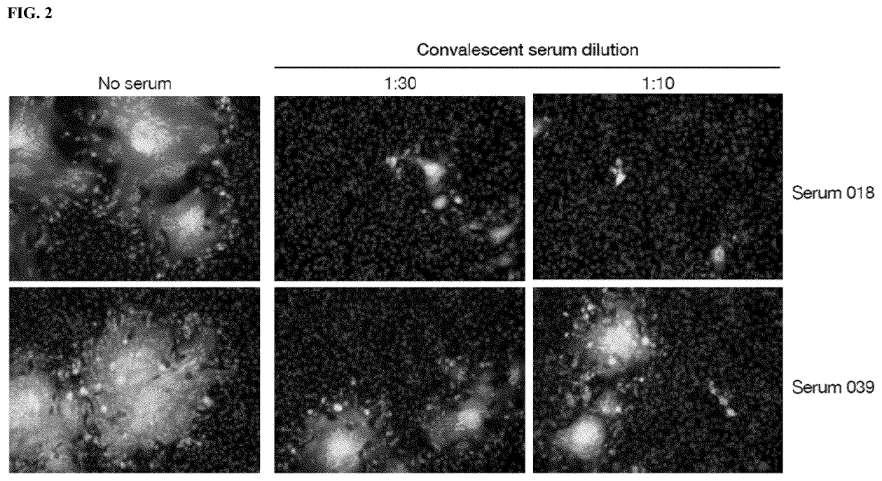

To generate a replication-competent rVSV expressing SARS-CoV-2 S, the open-reading frame of the native VSV entry glycoprotein gene, G, was replaced with that of the SARS-CoV-2 S (Wuhan-Hu-1 isolate) (FIG. 1A). A sequence encoding the enhanced green fluorescent protein (eGFP) was also introduced as an independent transcriptional unit at the first position of the VSV genome. Plasmid-based rescue of rVSV-SARS-CoV-2 S generated a slowly replicating virus bearing the wild-type S sequence. Five serial passages yielded viral populations that displayed enhanced spread. This was associated with a dramatic increase in the formation of syncytia (FIG. 1B and FIG. 2) driven by S-mediated membrane fusion (FIG. 2). Sequencing of this viral population identified nonsense mutations that introduced stop codons in the S glycoprotein gene (amino acid position C1250* and C1253*), causing 24- and 21-amino acid deletions in the S cytoplasmic tail, respectively. SΔ24 and SΔ21 were maintained in the viral populations upon further passage, and SΔ21 in all plaque-purified isolates, highlighting their likely importance as adaptations for viral growth. Viral population sequencing after four more passages identified two additional mutations, L517S and P812R in S1 and S2, respectively, whose emergence coincided with more rapid viral spread and the appearance of non-syncytium-forming infectious centers (FIG. 1B, passage 5). Pelleted viral particles from clarified infected-cell supernatants incorporated the S glycoprotein, as determined by an S-specific ELISA (FIG. 1C).

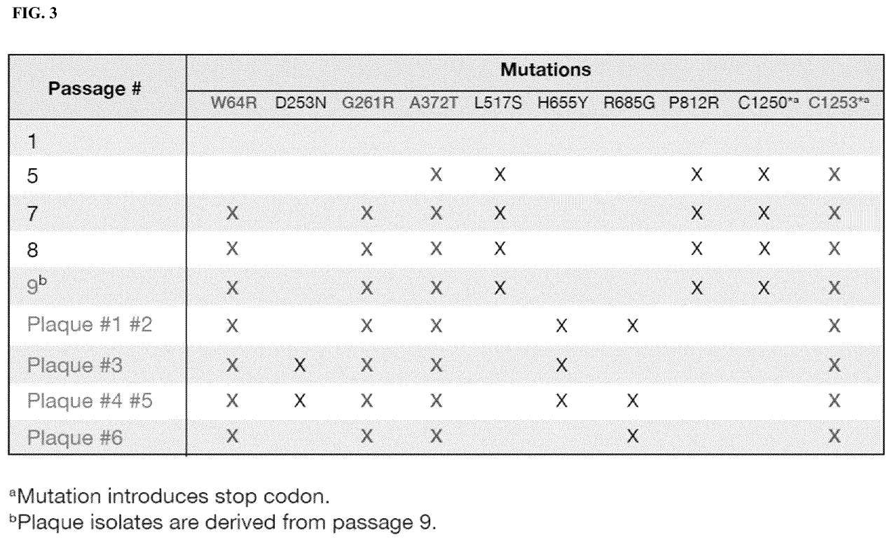

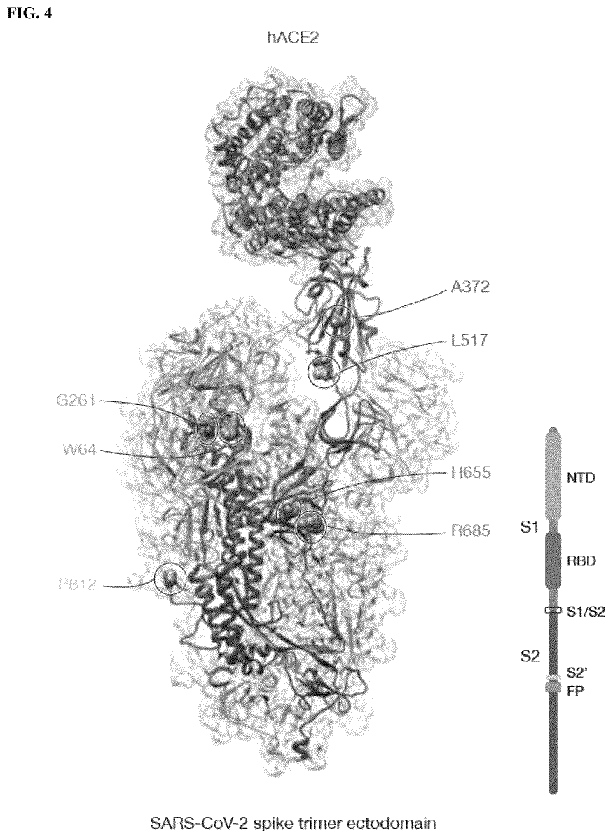

Six plaque-purified viral isolates derived from the passage 9 (P9) population were next sequenced. All of these viral clones bore the SΔ21 deletion in the S cytoplasmic tail and spread without much syncytia formation (FIG. 1D). Interestingly, all of these isolates contained three amino acid changes at S-glycoprotein positions other than 517 or 812-W64R, G261R, and A372T—in addition to the C-terminal SΔ21 deletion (FIG. 3 and FIG. 4). Five of the six isolates also contained mutations H655Y or R685G. Importantly, peak titers of all these viral isolates ranged between 1-3×10⁷ infectious units per mL (FIG. 1E), suggesting that the mutations they share (or a subset of these mutations) drive rVSV-SARS-CoV-2 S adaptation for efficient spread in tissue culture with little or no syncytium formation.

### Example 3: rVSV-SARS-CoV-2 S Entry is Cysteine Cathepsin-Dependent

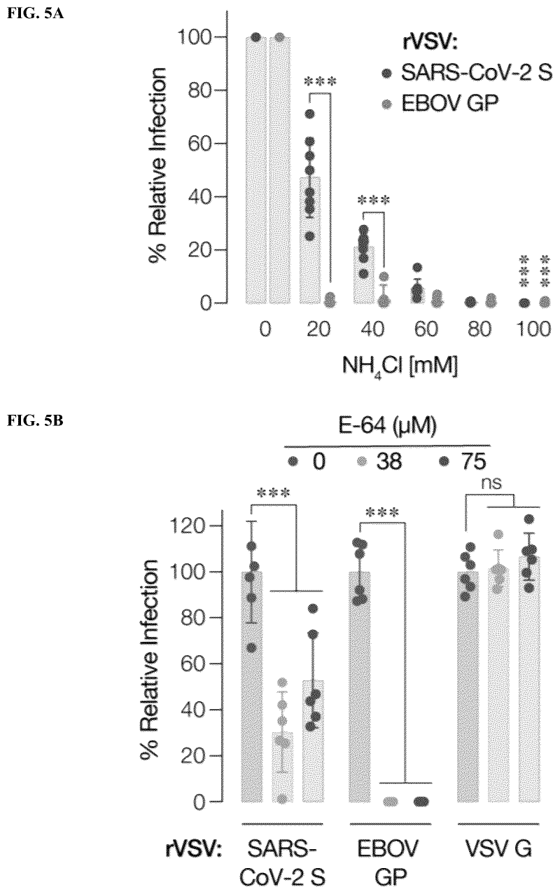

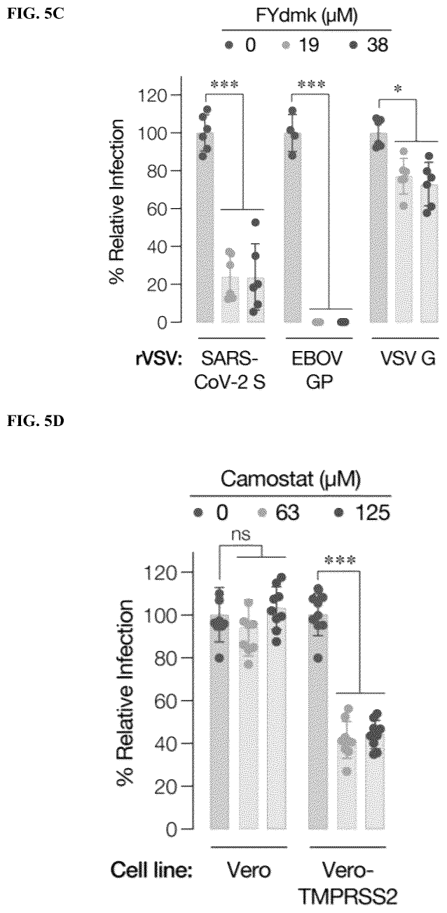

SARS-CoV-2 entry in cells has been shown to be dependent on the proteolytic activity of acid-dependent endosomal cysteine cathepsins, including cathepsin L (Hoffmann et al. (2020a) Cell 181:271-280.e8; Wang et al. (2020) Cell 181:894-904.e9). Accordingly, the effects of chemical inhibitors of cysteine cathepsins were tested on rVSV-SARS-CoV-2 S infection. Pretreatment of cells with NH₄Cl, an inhibitor of endosomal acidification, reduced entry by rVSVs bearing SARS-CoV-2 S or the Ebola virus glycoprotein (EBOV GP) in a dose-dependent manner (FIG. 5A). However, S-mediated entry was comparatively less sensitive to NH₄Cl than that by EBOV GP (FIG. 5A). Next, cysteine cathepsin inhibitors E-64 (FIG. 5B) and FYdmk (FIG. 5C) were tested. Pre-treatment of cells with both of these compounds also inhibited S-mediated entry, albeit with reduced sensitivity relative to that observed for EBOV GP-dependent entry (FIGS. 5B-5C). Together, these findings confirm that rVSV-SARS-CoV-2 S resembles the authentic agent in its requirements for endosomal acid pH and cysteine cathepsins. They also suggest a reduced dependence on these host factors for entry by SARS-CoV-2 S relative to EBOV GP, a model glycoprotein known to fuse in late endo/lysosomal compartments following extensive endosomal proteolytic processing.

### Example 4: TMPRSS2 can Mediate rVSV-SARS-CoV-2 S Entry

The Type II transmembrane serine protease TMPRSS2 plays a key role in the infection and spread of a number of enveloped viruses in cells of the human airway (Choi et al. (2009) Trends Mol. Med. 15:303-312; Shen et al. (2017) Biochimie 142:1-10). TMPRSS2 cleavage of the hemagglutinin spike precursors (HA0) of some influenza A and B viruses at a monobasic site generates HA1 and HA2 subunits, thereby priming HA for viral membrane fusion (Böttcher-Friebertshäuser et al. (2014) Curr. Top. Microbiol. Immunol. 385:3-34; Limburg et al. (2019) J. Virol. 93:e00649-19; Böttcher-Friebertshauser et al. (2010) J. Virol. 84:5605-5614; Chaipan et al. (2009) J. Virol. 83:3200-3211). TMPRSS2 can also activate membrane fusion by the spike glycoproteins of human coronaviruses, including those of SARS-CoV and MERS-CoV, by cleaving the spike at the monobasic S2′ site during entry (Kawase et al. (2012) J. Virol. 86:6537-6545; Zhou et al. (2015) Antiviral Res. 116:76-84). Recent work indicates that TMPRSS2 may play a similar role in SARS-CoV-2 entry into human airway and intestinal cells (Bestle et al. (2020) BioRxiv. doi:doi.org/10.1101/2020.04.15.042085; Hoffmann et al. (2020b) Mol. Cell 78:779-784.e5). Accordingly, the effect of the trypsin-like serine protease inhibitor camostat mesylate (camostat), previously shown to block TMPRSS2 catalytic activity and inhibit viral glycoprotein activation, was evaluated (Zhou et al. (2015) Antiviral Res. 116:76-84; Kawase et al. (2012) J. Virol. 86:6537-6545; Nimishakavi et al. (2015) PLoS ONE 10:e0141169), on rVSV-SARS-CoV-2 S infection. Pretreatment of Vero grivet monkey cells with camostat had little effect, consistent with their low expression levels of TMPRSS2 (Hoffmann et al. (2020b) Mol. Cell 78:779-784.e5). By contrast, camostat treatment significantly reduced VSV-SARS-CoV-2 S infection in Vero cells transduced to express human TMPRSS2 (Vero-TMPRSS2), as reported previously (Hoffmann et al. (2020a) Cell 181:271-280.e8) (FIG. 5D). These findings suggest that TMPRSS2 can promote cell entry by rVSV-SARS-CoV-2 S.

### Example 5: Human ACE2 is Required for rVSV-SARS-CoV-2 S Entry

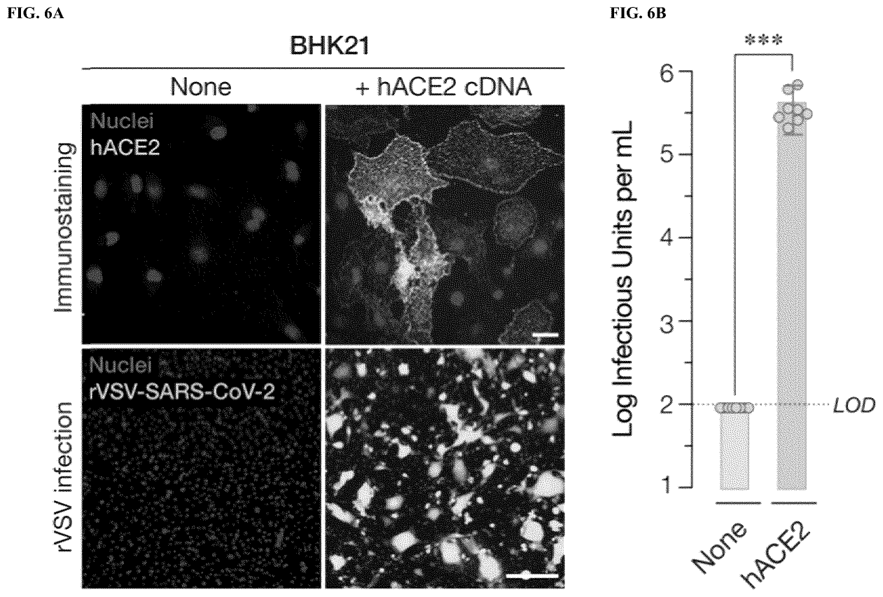

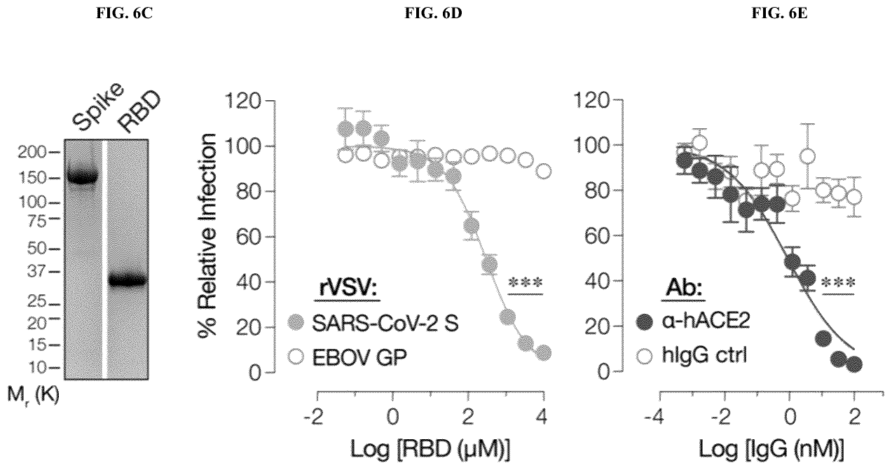

SARS-CoV-2 uses hACE2 as its entry receptor (Letko et aL (2020) Nat. Microbiol. 5:562-569; Shang et al. (2020a) Nature 581:221-224; Shang et al. (2020b) Proc Natl Acad Sci USA 117:11727-11734). Baby hamster kidney (BHK21) cells do not express detectable levels of ACE2 protein and are resistant to SARS-CoV-2 entry (Chu et al. 2020; Hoffmann et al. 2020a; Wang et al. 2020). Concordantly, no detectable infection by rVSV-SARS-CoV-2 S in BHK-21 cells was observed (FIG. 6A, top left). By contrast, BHK21 cells transduced to express hACE2 (FIG. 6A, top right) were highly susceptible to rVSV-SARS-CoV-2 S (FIG. 6A, bottom right and FIG. 6B).

To directly establish an entry-relevant interaction between rVSV-SARS-CoV-2 S and hACE2, the spike RBD was expressed and purified (FIG. 6C) and pre-incubated with target cells. RBD pre-treatment inhibited rVSV-SARS-CoV-2 S entry in a specific and dose-dependent manner (FIG. 6D). Moreover, pre-incubation of cells with an hACE2-specific mAb, but not an isotype-matched control mAb, potently abolished rVSV-SARS-CoV-2 S entry (FIG. 6E). These findings provide evidence that rVSV-SARS-CoV-2 S entry and infection, like that of the authentic agent, requires spike RBD-hACE2 engagement.

### Example 6: rVSV-SARS-CoV-2 S Infects Cells of Human Airway Origin in an ACE2-Dependent Manner

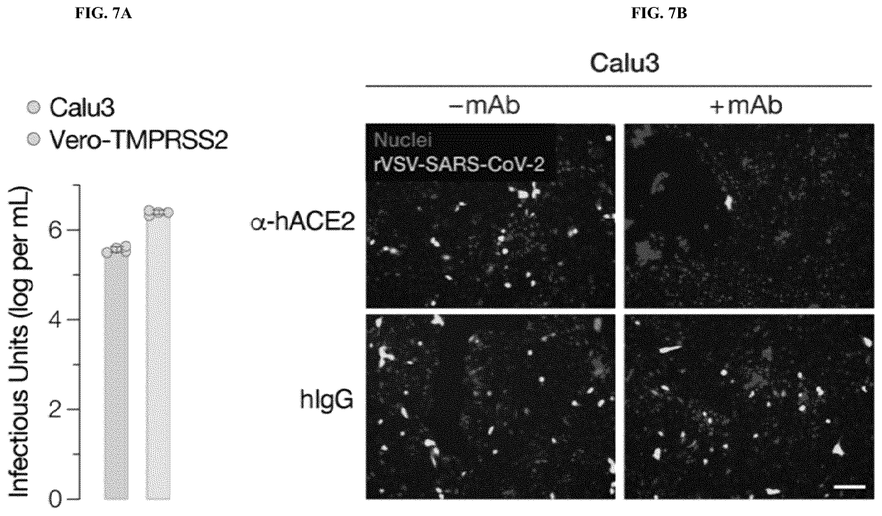

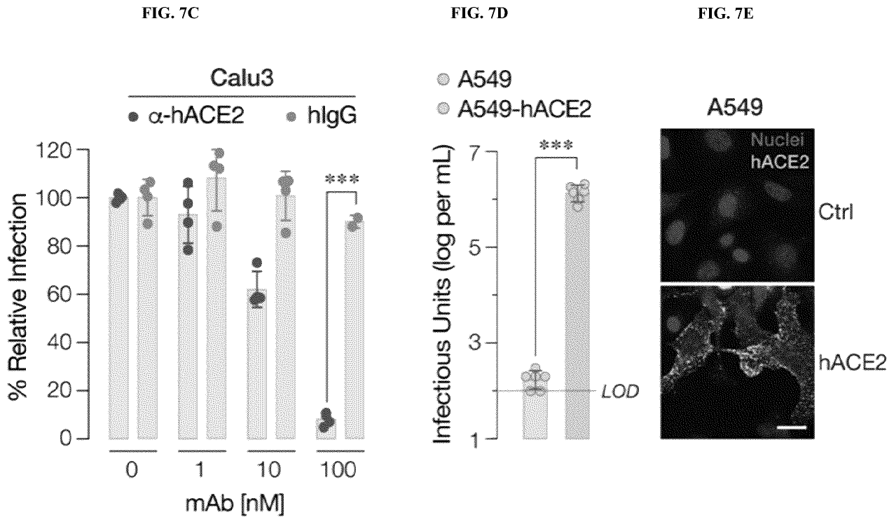

SARS-CoV-2 can infect multiple cell types in the human airway, including ciliated epithelial cells of the bronchial and bronchiolar mucosae and type I and II pneumocytes of the lung (Rockx et al. (2020) Science 368:1012-1015; Hui et aL (2020) Lancet Respir. Med. S2213-2600:30193-4). rVSV-SARS-CoV-2 entry and infection in epithelial cell lines that serve as models of human respiratory function was assessed. Specifically, it was found that the human lung adenocarcinoma cell line Calu3 was highly susceptible to infection (FIG. 7A) in a manner that was sensitive to treatment with a hACE2-specific mAb (FIGS. 7B-7C). By contrast, the human lung adenocarcinoma cell line A549 was refractory to infection (FIG. 7D), as previously documented with authentic SARS-CoV-2 and a single-cycle VSV vector bearing SARS-CoV-2 S (Chu et al. (2020) Clin. Infect. Dis. ciaa410; Harcourt et al. (2020) United States. 26:1266-1273; Hoffmann et al. (2020a) Cell 181:271-280.e8; Hui et al. (2020) Lancet Respir. Med. S2213-2600:30193-4). Because poorly differentiated A549 cells express little ACE2 (Jia et al. (2005) J. Virol. 79:14614-14621; Mossel et al. (2005) J. Virol. 79:3846-3850) (FIG. 7E), these cells were transduced to express hACE2 and then exposed them to virus. The A549-hACE2 cells were more susceptible than their parental cells by a factor of ≈10⁴ (FIG. 7E). Thus, rVSV-SARS-CoV-2 S can enter and infect cells of human airway origin in an ACE2-dependent fashion.

### Example 7: S Protein-Targeting Antibodies in COVID-19 Convalescent Sera Specifically Account for rVSV-SARS-CoV-2 S Neutralization

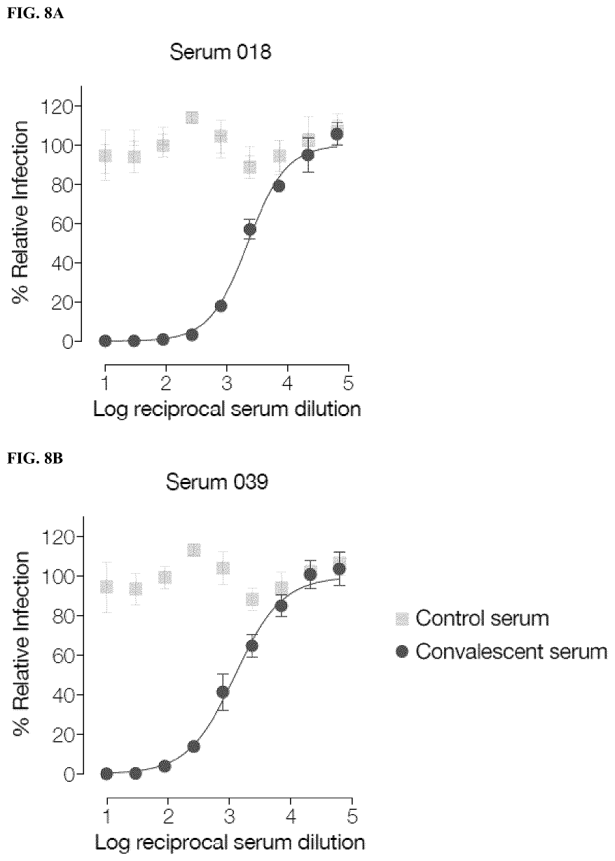

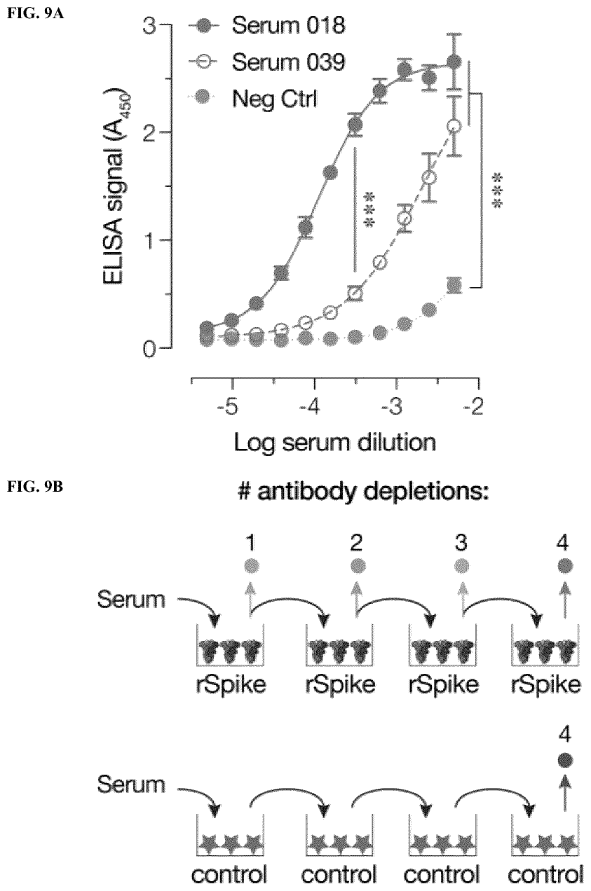

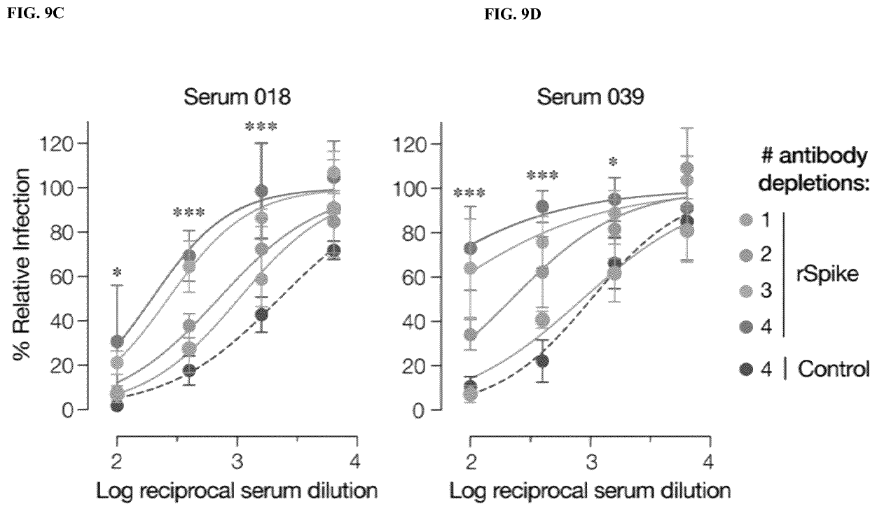

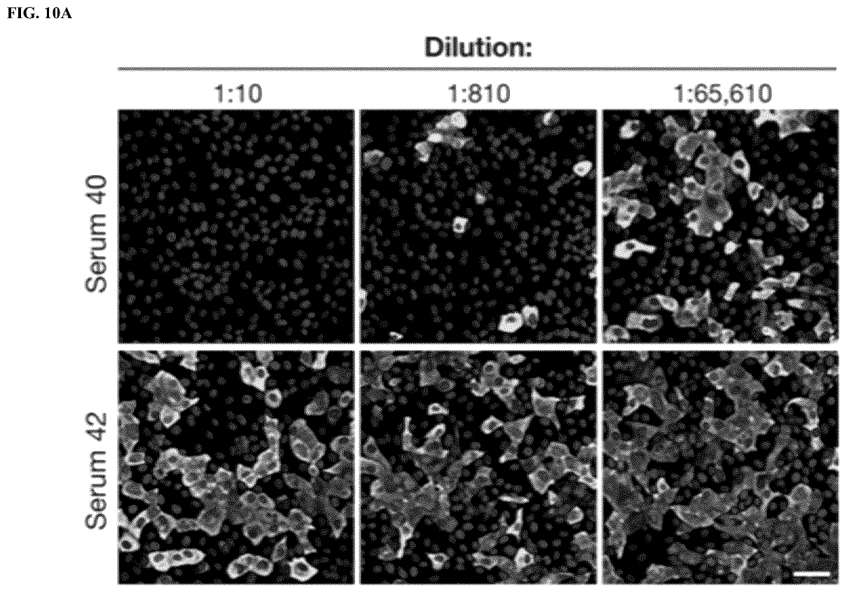

Prior to examining the performance of rVSV-SARS-CoV-2 S in neutralization assays with human antisera, a specific role for interaction between anti-spike antibodies in these sera and the VSV-borne S protein was sought to establish. Accordingly, the reactivity of two sera with rVSV-neutralizing activity (FIGS. 8A-8B) against viral particles was first evaluated by ELISA. Both sera specifically recognized rVSV-SARS-CoV-2 S particles (FIG. 9A) and were also shown to be reactive against a purified, trimeric preparation of the spike glycoprotein (Wrapp et al. (2020) Science 367:1260-1263). Further, serial pre-incubation of each serum with purified S immobilized on a high-binding plate depleted its capacity to inhibit rVSV-SARS-CoV-2 S infection to a degree that was commensurate with its content of S-specific antibodies (FIGS. 9B-9D). By contrast, parallel pre-incubations with blocked plates had little or no effect (FIGS. 9C-9D). These results indicate that S glycoprotein-targeting antibodies in COVID-19 convalescent sera specifically mediate rVSV-SARS-CoV-2 S neutralization.

### Example 8: The Susceptibilities of rVSV-SARS-CoV-2 S and Authentic SARS-CoV-2 to Antibody-Mediated Neutralization are Highly Correlated

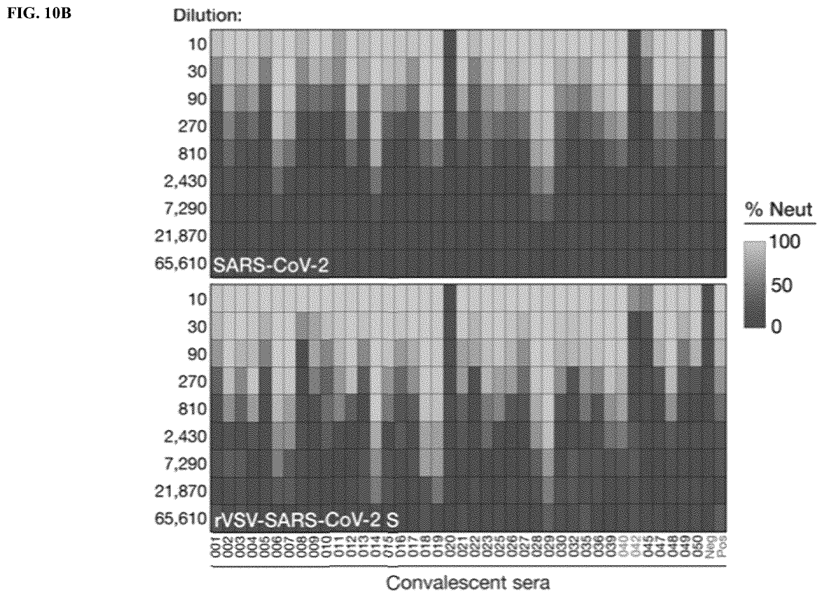

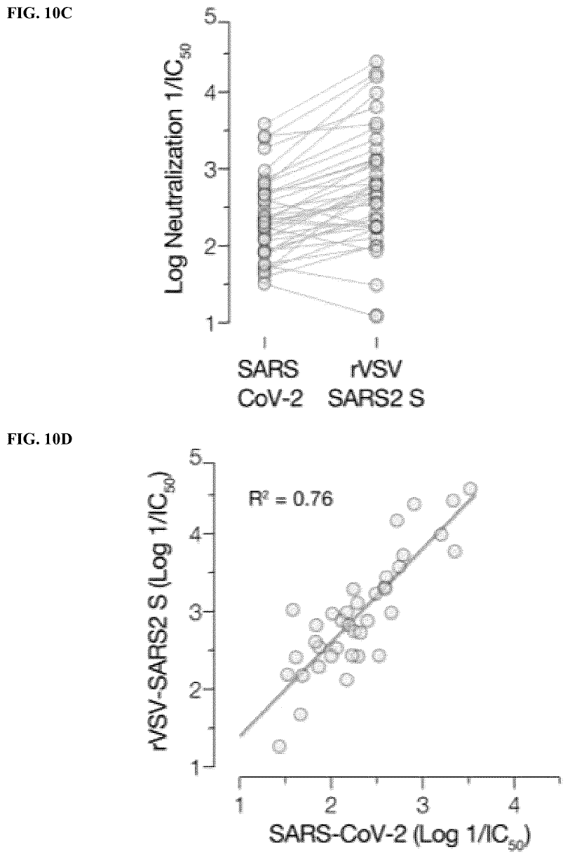

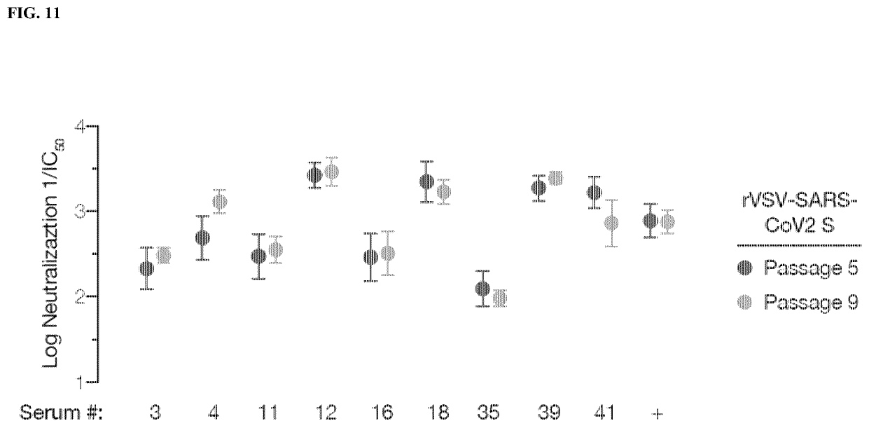

The capacities of human antisera derived from 40 COVID-19 convalescent donors to block infection by rVSV-SARS-CoV-2 S and authentic SARS-CoV-2 were compared in a microneutralization format. Briefly, pre-titrated amounts of viral particles were incubated with serial dilutions of each antiserum, and target cells were then exposed to the virus-antiserum mixtures. Viral infection was determined by enumerating eGFP-positive cells (rVSV) as above (FIGS. 1A-1E) or cells immunoreactive with a SARS-CoV-2 nucleocapsid protein-specific antibody (authentic virus) (FIG. 10A). Heatmaps of viral infectivity revealed similar antiserum donor- and dose-dependent neutralization patterns for rVSV-SARS-CoV-2 S and authentic SARS-CoV-2 (FIG. 10B). Comparison of the serum dilutions at half-maximal neutralization derived from logistic curve fits (neutralization IC₅₀) revealed a 3-10-fold shift towards enhanced neutralization with rVSV-SARS-CoV-2 S (FIG. 10C). The origin of this difference is unclear but does not appear to arise from viral passage-dependent changes in the rVSV-encoded spike gene sequence (FIG. 11). Rather, it may reflect assay-specific differences in the rVSV and authentic virus microneutralization formats employed herein. Nevertheless, the relative potencies of the antisera against rVSV-SARS-CoV-2 S and authentic SARS-CoV-2 were well correlated (R²=0.76) (FIG. 10D). In sum, these findings demonstrate the suitability of rVSV-SARS-CoV-2 S for rapid, high-throughout, reporter-based assays of spike glycoprotein-dependent entry and its inhibition.

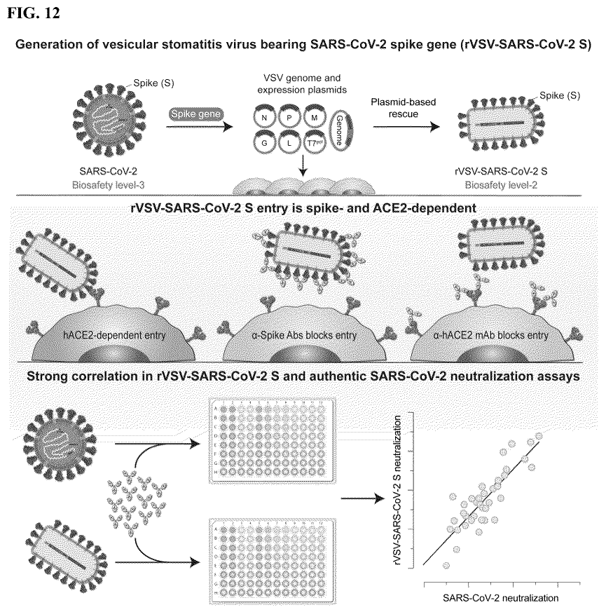

There is an urgent need for vaccines and therapeutics to prevent and treat COVID-19. The rapid development of SARS-CoV-2 countermeasures is contingent on the availability of robust, scalable, and readily deployable surrogate viral systems to screen antiviral humoral responses and define correlates of immune protection. Such tools would also facilitate the efficient down-selection of candidate antivirals and studies of their mechanisms of action. Here, a highly infectious recombinant vesicular stomatitis virus bearing the SARS-CoV-2 spike glycoprotein S that closely resembles the authentic agent in its entry-related properties was described (FIG. 12). It was shown that rVSV-SARS-CoV-2 S affords the high-throughput, reporter-based screening of small-molecule and antibody-based inhibitors targeting the viral spike glycoprotein with performance characteristics comparable to those of SARS-CoV-2 (FIG. 12).

rVSV-SARS-CoV-2 S initially replicated poorly in cell culture following its rescue from plasmids, but accelerated viral growth at passage 5 was noted (FIGS. 1A-1E). This coincided with the emergence of viral variants bearing S glycoproteins with 21- or 24-amino acid truncations of their cytoplasmic tails, as also observed by Case and co-workers (Case et al. (2020) Cell Host Microbe doi.org/10.1016/j.chom.2020.06.021). The cytoplasmic tails of the S glycoproteins of SARS-CoV and SARS-CoV-2 are highly similar and carry signals for their retention in the endoplasmic reticulum (ER), including a conserved K×H×× motif located near the C-terminus (McBride et al (2007) J. Virol. 81:2418-2428; Ujike et al (2016) J. Gen. Virol. 97:1853-1864). 18-19-amino acid deletions in the cytoplasmic tails of SARS-CoV S (Fukushi et al. (2005) J. Gen. Virol. 86:2269-2274; Fukushi et al. (2006) J. Med. Virol. 78:1509-1512) and SARS-CoV-2 S (Ou et al. (2020) Nat. Commun. 11:1620) have been shown to increase the infectivity of single-cycle VSV—S pseudotypes. As previously observed for ER/Golgi-localizing hantavirus glycoproteins (Slough et al. (2019) MBio 10:e02372-18), these deletions likely redistribute S glycoproteins to the cell surface, thereby relieving the spatial mismatch in budding between VSV and SARS-CoV2 (plasma membrane vs. ER, respectively) and enhancing S incorporation into VSV particles.

Accelerated growth by rVSV-SARS-CoV-2 S around passage 5 was accompanied by a marked increase in the occurrence of syncytia (see FIG. 1B) due to S-mediated cell-cell fusion (FIG. 2). This may reflect a functional property of the cytoplasmic tail-deleted S variants, including perturbations in their subcellular localization, as discussed above. Strikingly, passage 9 stocks and highly infectious viral plaque isolates from these stocks displayed a pattern of spreading infection more typical for rVSVs, with few large syncytia in evidence (FIG. 1B). In this regard, it is tempting to speculate that one or more additional S glycoprotein mutations detected in the passage 5-9 viral populations and in the six plaque isolates (FIG. 3) arose as compensatory changes to suppress the syncytiogenic propensity of the rVSV-encoded SΔ21 glycoprotein spikes. Indeed, several mutations in the S1 NTD and RBD may serve to modulate spike glycoprotein fusogenicity, as also may mutations near or at the S1-S2 cleavage site (H655Y and R685G, respectively) and/or at the S2′ cleavage site (P812R) (FIG. 3 and FIG. 4). Further, at least one mutation (H655Y), present in five of six rVSV-SARS-CoV-2 S plaque isolates, has arisen during natural SARS-CoV-2 evolution in humans (Yang et al. (2020) MedRxiv. doi.org/10.1101/2020.04.19.20071399), during transmission studies in a hamster model (Chan et al. (2020) Clin. Infect. Dis. ciaa325), and possibly during SARS-CoV-2 passage in tissue culture. The role(s) these mutations in the S glycoprotein ectodomain play in the maintenance of high levels of rVSV infectivity without the formation of large numbers of syncytia is investigated. These findings also highlight a feature of rVSV-SARS-CoV-2 S not shared by any of the viral entry surrogates described to date-its utility for forward genetics. This can be used to dissect structure-function relationships in the SARS-CoV-2 spike glycoprotein and to elucidate the mechanisms of action of spike- or entry-targeted antivirals.

It was demonstrated that rVSV-SARS-CoV-2 S can be used to rapidly and faithfully assess the neutralizing activities of large panels of COVID-19 convalescent sera (FIGS. 10A-10D) and spike-directed mAbs (Wec et al. (2020) Science doi: 10.1126/science.abc7424). The fidelity and high throughput of the rVSV-based 384-well plate microneutralization assay to rapidly pre-screen >300 COVID-19 convalescents have been exploited and potential convalescent plasma donors were identified first for the expanded access program and now for an ongoing randomized controlled trial of convalescent plasma therapy (Casadevall and Pirofski, (2020) J. Clin. Invest. 130:1545-1548). The utility and reliability of this approach is further enhanced by its synergy with the new SARS-CoV-2 microneutralization assay also described herein (FIGS. 10A-10B), which provides a rapid and non-subjective alternative to classical PRNT assays. When used in combination, these assays should afford the rapid mechanistic interrogation of cellular factors and antivirals that act at any step of the viral multiplication cycle-entry hits should affect both the rVSV and the authentic virus, whereas post-entry hits should affect only the latter.

As the COVID-19 pandemic continues apace and the development of plasma-, hyperimmune globulin-, mAb-, and small molecule-based countermeasures accelerates, the need for highly scalable viral assays continues to mount. The availability of highly infectious rVSV surrogates that can be scaled up with relative ease for antiviral screening and readily deployed in reporter-based microneutralization assays can facilitate these efforts.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

1. A recombinant vesicular stomatitis virus (VSV) vector comprising a nucleic acid encoding spike glycoprotein S of severe acute respiratory syndrome coronavirus-2 (SARS-CoV-2), or biologically active fragment thereof.

2. The recombinant VSV vector of claim 1, wherein the nucleic acid encoding a wild-type spike glycoprotein S of SARS-CoV-2, or biologically active fragment thereof.

3. The recombinant VSV vector of claim 2, wherein
(a) the wild-type spike glycoprotein S of SARS-CoV-2 comprises an amino acid sequence set forth in SEQ ID No: 1; or
(b) the nucleic acid comprises a nucleotide sequence set forth in SEQ ID No: 2.

4. (canceled)

5. The recombinant VSV vector of claim 1, wherein the nucleic acid encoding a mutated spike glycoprotein S of SARS-CoV-2, or biologically active fragment thereof.

6. The recombinant VSV vector of claim 5, wherein
(a) the mutated spike glycoprotein S of SARS-CoV-2 comprises mutations at positions corresponding to C1250, C1253, L517, P812, W64, D253, G261, A372, H655, and/or R685 of SEQ ID NO: 1;
(b) the mutated spike glycoprotein S of SARS-CoV-2 comprises one or more mutations selected from C1250*, C1253*, L517S, P812R, W64R, D253N, G261R, A372T, H655Y, and R685G;
(c) the mutations of the spike glycoprotein S are capable of facilitating replication of rVSV-SARS-CoV-2 S virus;
(d) the mutated spike glycoprotein S of SARS-CoV-2 comprises an amino acid sequence set forth in SEQ ID NOs: 3, 5, 7, 9, 11, or 13;
(e) the recombinant VSV vector comprises a nucleotide sequence set forth in SEQ ID No: 4, 6, 8, 10, 12, or 14; or
(f) the mutated spike glycoprotein S of SARS-CoV-2 comprises mutations C1253*, W64R, G261R, and A372T, optionally wherein (i) the mutated spike glycoprotein S of SARS-CoV-2 comprises an amino acid sequence set forth in SEQ ID No: 7, 9, 11, or 13; or (ii) the nucleic acid comprises a nucleotide sequence set forth in SEQ ID No: 8, 10, 12, or 14.

7-13. (canceled)

14. The recombinant VSV vector of claim 1,
(a) wherein the recombinant VSV vector lacks native VSV entry glycoprotein gene, G, optionally wherein the native VSV entry glycoprotein gene, G, is replaced with the nucleic acid encoding spike glycoprotein S of SARS-CoV-2; and/or
(b) further comprising a nucleic acid encoding a reporter, optionally wherein the reporter is a fluorescent protein, optionally an enhanced green fluorescent protein (eGFP).

15-18. (canceled)

19. A recombinant vesicular stomatitis virus (VSV) generated from the recombinant VSV vector of claim 1.

20. A recombinant vesicular stomatitis virus (VSV) comprising a genome which comprises a nucleic acid encoding spike glycoprotein S of SARS-CoV-2, or biologically active fragment thereof.

21. The recombinant VSV of claim 20, wherein the nucleic acid encoding a wild-type spike glycoprotein S of SARS-CoV-2, or biologically active fragment thereof.

22. The recombinant VSV of claim 21, wherein
(a) the wild-type spike glycoprotein S of SARS-CoV-2 comprises an amino acid sequence set forth in SEQ ID No: 1; or
(b) the nucleic acid comprises a nucleotide sequence set forth in SEQ ID No: 2.

23. (canceled)

24. The recombinant VSV of claim 20, wherein the nucleic acid encoding a mutated spike glycoprotein S of SARS-CoV-2, or biologically active fragment thereof.

25. The recombinant VSV of claim 24, wherein
(a) the mutated spike glycoprotein S of SARS-CoV-2 comprises mutations at positions corresponding to C1250, C1253, L517, P812, W64, D253, G261, A372, H655, and/or R685 of SEQ ID NO: 1;
(b) the mutated spike glycoprotein S of SARS-CoV-2 comprises one or more mutations selected from C1250*, C1253*, L517S, P812R, W64R, D253N, G261R, A372T, H655Y, and R685G;
(c) the mutations of the spike glycoprotein S are capable of facilitating replication of the recombinant VSV;
(d) the mutated spike glycoprotein S of SARS-CoV-2 comprises an amino acid sequence set forth in SEQ ID No: 3, 5, 7, 9, 11, or 13;
(e) the nucleic acid comprises a nucleotide sequence set forth in SEQ ID No: 4, 6, 8, 10, 12, or 14;
(f) the mutated spike glycoprotein S of SARS-CoV-2 comprises mutations C1253*, W64R, G261R, and A372T, optionally wherein (i) the mutated spike glycoprotein S of SARS-CoV-2 comprises an amino acid sequence set forth in SEQ ID No: 7, 9, 11, or 13; or (ii) the nucleic acid comprises a nucleotide sequence set forth in SEQ ID No: 8, 10, 12, or 14.

26-32. (canceled)

33. The recombinant VSV of claim 20, wherein
(a) the genome does not comprise native VSV entry glycoprotein gene, G, optionally wherein the native VSV entry glycoprotein gene, G, is replaced with the nucleic acid encoding spike glycoprotein S of SARS-CoV-2;
(b) the genome further comprises a nucleic acid encoding a reporter, optionally wherein the reporter is a fluorescent protein, optionally an enhanced green fluorescent protein (eGFP).

34-37. (canceled)

38. The recombinant VSV of claim 20, wherein
(a) the recombinant VSV viral particle incorporates the spike glycoprotein S of SARS-CoV-2;
(b) the recombinant VSV is replication-competent;
(c) the recombinant VSV is capable of efficient spread in tissue culture with little or no syncytium formation;
(d) entry of the recombinant VSV in cells depends on level and/or activity of cysteine cathepsins, and/or endosomal acid pH of the cells, optionally wherein the entry of the recombinant VSV in cells is reduced by NH4Cl, E-64, and FYdmk;
(e) entry of the recombinant VSV in cells depends on level and/or activity of TMPRSS2 of the cells, optionally wherein the entry of the recombinant VSV in cells is reduced by camostat mesylate;
(f) entry of the recombinant VSV in cells depends on level and/or activity of ACE2 of the cells, and/or interaction between the spike glycoprotein S and the ACE2 receptor, optionally wherein (i) the entry of the recombinant VSV in cells is reduced by a receptor-binding domain (RBD) of the spike glycoprotein S or an ACE2-specific antibody, (ii) the ACE2 is a human ACE2, and/or (iii) cells are of human airway origin; and/or
(g) the recombinant VSV is attenuated.

39-49. (canceled)

50. An immunogenic composition, a kit, or a device comprising the recombinant VSV vector of claim 1, optionally further comprising an adjuvant and/or a pharmaceutically acceptable carrier.

51-55. (canceled)

56. An immunogenic composition, a kit, or a device comprising the recombinant VSV of claim 20, optionally further comprising an adjuvant and/or a pharmaceutically acceptable carrier.

57-61. (canceled)

62. A method for identifying an agent that prevents and/or treats SARS-CoV-2 infection comprising:
a) contacting cells with the recombinant VSV vector of claim 1 or a VSV comprising the VSV vector of claim 1, and a test agent; and
b) identifying the test agent that reduces the recombinant VSV infection of the cells, thereby identifying an agent that prevents and/or treats SARS-CoV-2 infection.

63-78. (canceled)

79. A method of determining whether a subject has exposure to and/or protection from SARS-CoV-2 comprising:
a) contacting cells with the recombinant VSV vector of claim 1 or a VSV comprising the VSV vector of claim 1, and a sample obtained from the subject; and
b) determining the recombinant VSV infection of the cells;
wherein the absence of or a lower level of the recombinant VSV infection of the cells compared to a control level indicates that the subject has exposure to and/or protection from SARS-CoV-2.

80-86. (canceled)

87. A method of preventing and/or treating SARS-CoV-2 infection in a subject comprising administering to the subject a therapeutically effective amount of an immunogenic composition comprising the VSV vector of claim 1 or a VSV comprising the VSV vector of claim 1.

88. The method of claim 87, wherein
(a) the immunogenic composition is capable of eliciting an immune response in a subject;
(b) the administered immunogenic composition induces production of antibodies to the spike glycoprotein S of SARS-CoV-2;
(c) the administered immunogenic composition induces an immune response against the SARS-CoV-2 in the subject; and/or
(d) the subject is a mammal, optionally a human, a primate, or a rodent, optionally a human.

89-93. (canceled)


SEQUENCE LISTING (15 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 3822 bp, Severe acute respiratory syndrome coronavirus 2
  source 1..3822: mol_type = genomic DNA
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacatggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcgacagcagcagcggatggaccgccggagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcgcctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagcacgtgaataacagttacgagtgcgacatccctatcggcgccggcatctgtgccagctaccagacccagacaaacagccctagacgggctagatctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggacaaggtggaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagtacatcaagtggccttggtacatctggctgggattcatcgccggactgatcgccatcgtgatggtgaccatcatgctgtgctgtatgaccagctgctgcagttgcctgaagggctgttgcagctgcggcagctgctgcaagttcgacgaagatgactctgagcctgtgctgaagggcgtgaagctgcactacacctga

SEQ ID NO: 3 - protein, 1249 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSTSFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFESLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKRSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCS

SEQ ID NO: 4 - DNA, 3822 bp, synthetic construct
  source 1..3822: mol_type = other DNA
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacatggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcgacagcagcagcggatggaccgccggagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcacctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagtcactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagcacgtgaataacagttacgagtgcgacatccctatcggcgccggcatctgtgccagctaccagacccagacaaacagccctagacgggctagatctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcgtagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggacaaggtggaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagtacatcaagtggccttggtacatctggctgggattcatcgccggactgatcgccatcgtgatggtgaccatcatgctgtgctgtatgaccagctgctgcagttgcctgaagggctgttgcagctgaggcagctgatgcaagttcgacgaagatgactctgagcctgtgctgaagggcgtgaagctgcactacacctga

SEQ ID NO: 5 - protein, 1249 aa, synthetic construct
MFVFLVLLPLVSSQCVNLTTRTQLPPAYTNSFTRGVYYPDKVFRSSVLHSTQDLFLPFFSNVTRFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTARAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSTSFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFESLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKRSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCS

SEQ ID NO: 6 - DNA, 3822 bp, synthetic construct
  source 1..3822: mol_type = other DNA
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacacggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcgacagcagcagcggatggaccgccagagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcacctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagtcactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagcacgtgaataacagttacgagtgcgacatccctatcggcgccggcatctgtgccagctaccagacccagacaaacagccctagacgggctagatctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcgtagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggacaaggtggaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagtacatcaagtggccttggtacatctggctgggattcatcgccggactgatcgccatcgtgatggtgaccatcatgctgtgctgtatgaccagctgctgcagttgcctgaagggctgttgcagctgaggcagctgatgcaagttcgacgaagatgactctgagcctgtgctgaagggcgtgaagctgcactacacctga

SEQ ID NO: 7 - protein, 1252 aa, synthetic construct
MFVFLVLLPLVSSQCVNLTTRTQLPPAYTNSFTRGVYYPDKVFRSSVLHSTQDLFLPFFSNVTRFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTARAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSTSFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEYVNNSYECDIPIGAGICASYQTQTNSPRRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGS

SEQ ID NO: 8 - DNA, 3822 bp, synthetic construct
  source 1..3822: mol_type = other DNA
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacacggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcgacagcagcagcggatggaccgccagagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcacctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagtacgtgaataacagttacgagtgcgacatccctatcggcgccggcatctgtgccagctaccagacccagacaaacagccctagacgggctggatctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggacaaggtggaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagtacatcaagtggccttggtacatctggctgggattcatcgccggactgatcgccatcgtgatggtgaccatcatgctgtgctgtatgaccagctgctgcagttgcctgaagggctgttgcagctgcggcagctgatgcaagttcgacgaagatgactctgagcctgtgctgaagggcgtgaagctgcactacacctga

SEQ ID NO: 9 - protein, 1252 aa, synthetic construct
MFVFLVLLPLVSSQCVNLTTRTQLPPAYTNSFTRGVYYPDKVFRSSVLHSTQDLFLPFFSNVTRFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGNSSSGWTARAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSTSFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGS

SEQ ID NO: 10 - DNA, 3822 bp, synthetic construct
  source 1..3822: mol_type = other DNA
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacacggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcaacagcagcagcggatggaccgccagagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcacctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagtacgtgaataacagttacgagtgcgacatccctatcggcgccggcatctgtgccagctaccagacccagacaaacagccctagacgggctagatctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggacaaggtggaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagtacatcaagtggccttggtacatctggctgggattcatcgccggactgatcgccatcgtgatggtgaccatcatgctgtgctgtatgaccagctgctgcagttgcctgaagggctgttgcagctgcggcagctgatgcaagttcgacgaagatgactctgagcctgtgctgaagggcgtgaagctgcactacacctga

SEQ ID NO: 11 - protein, 1252 aa, synthetic construct
MFVFLVLLPLVSSQCVNLTTRTQLPPAYTNSFTRGVYYPDKVFRSSVLHSTQDLFLPFFSNVTRFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGNSSSGWTARAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSTSFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEYVNNSYECDIPIGAGICASYQTQTNSPRRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGS

SEQ ID NO: 12 - DNA, 3822 bp, synthetic construct
  source 1..3822: mol_type = other DNA
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacacggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcaacagcagcagcggatggaccgccagagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcacctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagtacgtgaataacagttacgagtgcgacatccctatcggcgccggcatctgtgccagctaccagacccagacaaacagccctagacgggctggatctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggacaaggtggaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagtacatcaagtggccttggtacatctggctgggattcatcgccggactgatcgccatcgtgatggtgaccatcatgctgtgctgtatgaccagctgctgcagttgcctgaagggctgttgcagctgcggcagctgatgcaagttcgacgaagatgactctgagcctgtgctgaagggcgtgaagctgcactacacctga

SEQ ID NO: 13 - protein, 1252 aa, synthetic construct
MFVFLVLLPLVSSQCVNLTTRTQLPPAYTNSFTRGVYYPDKVFRSSVLHSTQDLFLPFFSNVTRFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTARAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSTSFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGS

SEQ ID NO: 14 - DNA, 3822 bp, synthetic construct
  source 1..3822: mol_type = other DNA
atgttcgtgttcctggtgctcctgcctctggtgagcagccagtgcgtgaacctgaccacccgaacccagctcccaccagcctacaccaacagctttacacggggcgtgtactaccctgacaaggtgttcagatctagcgtcctgcacagcactcaggacctcttcctgccgttcttcagcaacgtgacacggttccacgccatccacgtgagcggcacaaacggaaccaagcggtttgataaccccgtcctgccattcaatgatggagtttacttcgccagtaccgagaagagtaacatcatccggggctggatcttcggcaccaccctggatagcaaaacacagagcctcctgatcgtgaacaatgccacgaacgtcgtgatcaaggtgtgcgagttccagttttgcaatgatcctttcctgggtgtgtactaccacaagaacaacaagagctggatggaaagcgagttcagagtctacagcagcgccaacaactgcacattcgagtacgtctctcagccttttctgatggaccttgaggggaaacaaggcaacttcaagaacctgagagaattcgtgttcaagaacatcgacggctacttcaaaatctactccaagcacacacccatcaacctggtccgggacctccctcagggcttcagcgccctggaacccctggtcgacctgcccataggcatcaacataacgcggttccaaaccctgctggccctgcatagatcctacctgactcctggcgacagcagcagcggatggaccgccagagctgcagcctactatgtgggctacctgcaacctagaaccttcctgctgaagtacaacgagaacggcacaatcacagacgccgtcgactgcgccctggaccctctctctgagacaaagtgcaccctgaagtccttcaccgtggaaaagggcatctaccagaccagcaacttccgggtgcagcctacagagagcatcgtgcgatttccaaacattaccaacctctgccccttcggcgaggtgtttaacgccacaagatttgcctccgtttacgcctggaatagaaagagaatcagcaattgtgtggccgactactccgtgctgtataacagcacctctttcagcaccttcaagtgctacggcgtttccccaacaaagctgaatgacctgtgcttcaccaacgtgtacgccgactccttcgtaattagaggcgatgaggtgcggcagatcgcaccaggccagaccggtaagatcgctgactacaactataagctgcctgatgattttacaggctgcgtgatcgcctggaactctaacaacctggatagcaaggtgggcggcaactacaactacctgtaccggctgtttcgcaagtctaacctgaaacctttcgagagagacatctccacagagatctaccaggccggttctacaccttgtaacggggtggaaggcttcaactgttacttccctctgcaaagctacggcttccagcctaccaatggagtcggctaccagccataccgggtggtcgtgctgtccttcgagttactccacgcccccgccaccgtctgcggtcctaagaagtccaccaatctggttaagaacaaatgcgtgaacttcaacttcaacggcctgaccgggaccggcgtgctgaccgaaagcaacaaaaagttcctccccttccagcagttcggccgtgatatcgctgacaccacagatgccgtcagagatccacagaccctggaaatcctggatattacaccctgctccttcggaggagtttctgtgatcacccccgggaccaataccagcaaccaggtggctgtgctgtaccaagatgttaactgcaccgaggttcctgtggccatccacgccgatcagctgacacctacttggagagtgtactccactggctccaatgtgttccagaccagggccggatgtctgatcggcgccgagcacgtgaataacagttacgagtgcgacatccctatcggcgccggcatctgtgccagctaccagacccagacaaacagccctagacgggctggatctgtagctagccagagcatcatcgcctacaccatgagcctgggcgcagagaacagcgtggcctattccaacaactctatcgccattcccaccaactttacaattagcgtcacaacagagatcctgcccgtgagcatgaccaagaccagcgtggactgtacaatgtacatctgtggcgacagcactgaatgcagcaacctgctgctgcaatacggctccttttgcacccaactgaaccgggcgctgaccggaatcgccgtggaacaggacaaaaatacccaggaggtgttcgcccaagtgaagcagatctacaagaccccacctatcaaggacttcggcggctttaactttagccagattctccctgatccttctaagcctagcaagcggagctttatcgaggatctgctgttcaacaaggtcaccctggccgatgccggctttatcaaacagtatggcgattgcctgggcgacatagccgccagagatctgatctgcgcccagaaattcaacggcctgacagttctcccacctctgctgaccgacgagatgatcgctcagtacacctctgccctgctggctggcaccatcacatctgggtggacatttggcgccggcgccgccctgcagatcccctttgccatgcagatggcctatagattcaacggaatcggcgtgacccagaacgtgctgtatgaaaaccagaagctgatcgctaaccagttcaattctgccatcggcaagatccaggactccctctcctctaccgccagcgccctgggcaaactgcaggacgtggtgaatcagaacgcccaagccctgaacaccctggtgaagcagctcagcagcaattttggcgccatcagctctgtgctgaacgatatcctgtctagactggacaaggtggaagccgaagtccagatcgatagactgatcacaggcagactgcagtccctgcaaacctacgtgacccaacagctgatcagggccgctgaaataagagccagcgccaatctcgccgctaccaagatgtccgagtgtgtgctgggacagtctaaacgcgttgacttctgcggcaaaggctatcacctgatgagcttcccccagagcgcgccgcacggcgtggtgttcctgcatgtgacatacgtgcctgcccaagagaagaatttcacaaccgcccctgccatctgccacgacggcaaggcccacttcccaagagagggcgttttcgtttccaatggcacacactggttcgtgacacaaagaaacttctacgaaccccagattatcaccaccgacaacaccttcgtgagtggcaattgtgacgtggtcatcggaatcgtgaacaacacagtgtacgaccctctgcaacctgagctggactcttttaaggaagagctggacaagtactttaaaaaccacaccagccccgatgtggacctgggcgacatcagtggcattaacgccagcgtggtgaacatccaaaaggaaatcgacagactgaacgaggtggccaagaacctgaacgagtccctgatcgacctgcaggagctcggcaaatacgagcagtacatcaagtggccttggtacatctggctgggattcatcgccggactgatcgccatcgtgatggtgaccatcatgctgtgctgtatgaccagctgctgcagttgcctgaagggctgttgcagctgcggcagctgatgcaagttcgacgaagatgactctgagcctgtgctgaagggcgtgaagctgcactacacctga

SEQ ID NO: 15 - protein, 20 aa, synthetic construct
CKFDEDDSEPVLKGVKLHYT